SEQUENCE LISTING (91 sequences)

SEQ ID NO: 1 - protein, 225 aa, Chromohalobacter salexigens
MADSSPLIIEPRQARAADATVILLHGLGADGHDFEPLVPALPLAKDLAVRFVLPHAPRMPVTVNGGMEMPAWYDILDMNLGRRIDEAQLKASADMVHGLIDAEIARGIDSRRIIVAGFSQGGAVAYHAALTYPKPLGGLLALSTYFATATSIEPSEANRALPIEVHHGSFDPVVPEALGHEGAERAEALGYAVTYRTYPMQHALCPEQIEDIGQWLNARLGAKEA

SEQ ID NO: 2 - protein, 218 aa, Pseudomonas fluorescens
MTEPLILQPAKPADACVIWLHGLGADRYDFMPVAEALQESLLTTRFVLPQAPTRPVTINGGYEMPSWYDIKAMSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 3 - protein, 218 aa, Pseudomonas aeruginosa
MSEPLILDAPNADACIIWLHGLGADRTDFKPVAEALQMVLPSTRFILPQAPSQAVTVNGGWVMPSWYDILAFSPARAIDEDQLNASADQVIALIDEQRAKGIAAERIILAGFSQGGAVVLHTAFRRYAQPLGGVLALSTYAPTFDDLALDERHKRIPVLHLHGSQDDVVDPALGRAAHDALQAQGVEVGWHDYPMGHEVSLEEIHDIGAWLRKRLGGS

SEQ ID NO: 4 - protein, 309 aa, Sulfolobus solfataricus
MPLDPEVRNFLQVYYKANIIDFTKYQFQEIRQKVNELLAKAVPKDPVGETRDMKIKLEDYELPIRIYSPIKRTNNGLVMHFHGGAWILGSIETEDAISRILSNSCECTVISVDYRLAPEYKFPTAVYDCFNAIVWARDNAGELGIDKDKIATFGISAGGNLVAATSLLARDNKLKLTAQVPVVPFVYLDLASKSMNRYRKGYFLDINLPVDYGVKMYIRDEKDLYNPLFSPLIAEDLSNLPQAIVVTAEYDPLRDQGEAYAYRLMESGVPTLSFRVNGNVHAFLGSPRTSRQVTVMIGALLKDIFKGSS

SEQ ID NO: 5 - protein, 281 aa, Aspergillus niger
MKQFSAKYALILLATAGQALAASTQGISEDLYNRLVEMATISQAAYADLCNIPSTIIKGEKIYNAQTDINGWILRDDTSKEIITVFRGTGSDTNLQLDTNYTLTPFDTLPQCNDCEVHGGYYIGWISVQDQVESLVKQQASQYPDYALTVTGHSLGASMAALTAAQLSATYDNVRLYTFGEPRSGNQAFASYMNDAFQVSSPETTQYFRVTHSNDGIPNLPPADEGYAHGGVEYWSVDPYSAQNTFVCTGDEVQCCEAQGGQGVNDAHTTYFGMTSGACTW

SEQ ID NO: 6 - protein, 222 aa, Francisella tularensis
MNYELMEPAKQARFCVIWLHGLGADGHDFVDIVNYFDVSLDEIRFIFPHADIIPVTINMGMQMRAWYDIKSLDANSLNRVVDVEGINSSIAKVNKLIDSQVNQGIASENIILAGFSQGGIIATYTAITSQRKLGGIMALSTYLPAWDNFKGKITSINKGLPILVCHGTDDQVLPEVLGHDLSDKLKVSGFANEYKHYVGMQHSVCMEEIKDISNFIAKTFKI

SEQ ID NO: 7 - protein, 270 aa, unidentified
  REGION 1..270: note = Description of Unknown: Uncultured bacterium FLS12
MALFQCDFFSDVLGLSTSMTVILPQETTGQIGMAGGSERREHPTLFLLHGLSDDHTIWLRRTSIERYVAEMGLAVVMPAVHRSFYTDMAHGLQYWTFISEELPALARSFFPLATAREDTFVAGLSMGGYGALKLGMRHPERFAAAASLSGALDITFDPAEHIAMEDDVWVAEQRNIFGDLAALPGSDHDLFALAERMAQSDGPVPKLYQCCGTEDFLYEDNVRFRDHVRGLGLDFMYEESPGEHEWGYWDAQIQRVLAWLPLRPPGTAPA

SEQ ID NO: 8 - protein, 219 aa, Pelagibacterium halotolerans
MTEPVKLSGPMLPAVSGAAKSLVVLLHGYGSDGRDLIALGQFWRDSFPDTMFVAPNAPHVCGGNPFGYEWFPLDLERDRTLARLAGAETAHPVLDAFLADLWAQTGLGPADTILVGFSQGAMMALYTGLRLPEPLKAIIAFSGLIVAPEKLEAEIASKPPVLLIHGDLDDVVPVIGSETALPKLIDLGIDARLHISQGSGHTIAQDGLDTATAFLREIL

SEQ ID NO: 9 - protein, 306 aa, Sulfolobus islandicus
MMPLDPRIKELLESGFIVPIGKASVDEVRKIFRQLASAAPKVEVGKVEDIKIPGSEANINARVYLPKANGPYGVLIYLHGGGFVIGDVESYDPLCRAITNACNCVVVSVDYRLAPEYKFPSAVIDSFDATNWVYNNLDKFDGKMGVAIAGDSAGGNLAAVVALLSKGKLNLKYQILIYPAVGFDSVSRSMIEYSDGFFLTREHIEWFGSQYLRSPADLLDFRFSPILAQDLSGLPPALIITAEYDPLRDQGEAYANRLLQAGVPVTSVRFNNVIHGFLSFFPLIEQGRDAISLIGSVLRRTFYDKS

SEQ ID NO: 10 - protein, 221 aa, Halomonas sp.
MTAPGELIIEPKDGQPADACVFIIHGLGADGHDFEPLVPALALPKDSRVRFIMPHAPRLPVTINGGMVMPAWYDILAMDLGRRVDERQLKQSAERIQALIQEQIDQGIDSQRIIVAGFSQGGAVAYHAALTFPAPLGGLLAMSTYFATADNIDLAEANRQIPIEVQHGNFDPIVPESLGRSGADRLKEMGYAVNYRQYPMAHALCPQQVNDIGKWLSARLN

SEQ ID NO: 11 - protein, 219 aa, Ignatzschineria indica
MDKPIILDPKQSADSAVIWLHGLGATKEDFLPVAQILQRDALPHTRFILPQAPVRPVTLNNGFPMPSWYDIIALTSPREIKLSELDESSQSIIALIEAEIEKGIPLERIILAGFSQGGAVVLHTAFIAYPKNVGGVMALSTYSATFDEAITLDEKKKQIPTLHLHGSLDPVVKIELGRAAEQFLKAQGIDTRWHDYPMQHEVINDELQDIAKWLIERLG

SEQ ID NO: 12 - protein, 217 aa, Pseudomonas citronellolis
MSQPLLLEPTQPADSCVIWLHGLGADRYDFEPVARMLQKVLPRTRFILPQAPTRPVTVFNGMPAPSWYDIKAMAPARAIDEAQLDASADAVIALIEGQLAEGIAQRRIVLAGFSQGGAVVLHTGYLRWPGELGGVMALSTYGPTFDDDLQLPEAKKQQPALCLHGTYDDVVAPAMGRAAYDFLQRQGVAVQWRDYPMAHEVSNQEIADIAAWLRERL

SEQ ID NO: 13 - protein, 486 aa, Myxococcus fulvus
MNGLRWRLTGVVMAWVLVAPLAEAANVKQEVDRYISGFHQKGLFNGTVLVANERGILLKKGYGAANLEWKVPNAPDTKFRIGSITKSFTATVILQLAAEGKLQLDDPITKHLPDYRKDTGDRVTITHLLNHTSGIPSYTSKPAIMKDADGFESVAAFVKKACSDDLEFEPGTKYAYNNSGYFLLGAIIEKLTGQTYAEAVQARILGPLGMKDTGYDVSATVLPKRASGYAQAPGGIVNAAWLDMNLPYAAGSLYSTVEDLYRWERAFHGDTLLPAALKQKMLTPGLAHYGFGWVMSDMTLHDGKTKLPGIFHTGGINGFSSILVRVPERKEAVILLDNMTHGGLQELAGGVLSILHGLTPRPARMPIGNVMMESLGKGSVAQAIATYRTLKKTKQAEYDFSERHLNTVGYHLLRSGRAADAIEVFKLNVEMFPEAANCHDSLGEAYAAHGDKARAITSYRKALELAPKNEHAVKMLEQLEEPAAKR

SEQ ID NO: 14 - protein, 218 aa, Pseudomonas saudiphocaensis
MTDPLIIEPAQTADSCVIWLHGLGADRYDFQPVAEMLQQRLLHTRFVLPQAPTRAVTINGGWAMPSWYDIQAMSPARAIDQAQLEQSAQTVIELIEQQRDSGIDPRRIFLAGFSQGGAVVYHTAFLRWAGPLGGVLALSTYAPTFGDDLKLSPLQAGLPVLCLHGSRDDVVPPAMGRAAHDCLQQNQVQTQWKEYPMAHEVQPTEIQDIGDWLASRLG

SEQ ID NO: 15 - protein, 222 aa, Ventosimonas gracilis
MTEPLIIEPSQPADSAVIWLHGLGADRFDFEPVARLLGQHLPSTRFILPQAPTRPVTFNMGHAMPSWYDILALDGSERAINPADLEASSETLIALINAQQQSGIDSKRIVLAGFSQGGAVVLHTALLRFDEKLAGVLALSTYAPTFNAETQFAESKQNLPVLCMHGSEDAVLPISMGRAVYDKLSEQGIKANWRDYPMGHEVRPEQLRDILDWLKNTLPSLP

SEQ ID NO: 16 - protein, 218 aa, Pseudomonas fluorescens
MTEPLILQPAKPADACVIWLHGLGADRYDFMPVAEALQESLLTTRFVLPQAPTRPVTINGGYEMPSWYDIKAHSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 17 - protein, 218 aa, Pseudomonas fluorescens
MTEPLILQPAKPADACVIWLHGLGADRYGFMPVAEALQESLLTTRFVLPQAPTRPVTINGGYEMPSWYDIKAMSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 18 - protein, 218 aa, Pseudomonas fluorescens
MTEPLILQPAKPADACVIWLHGLGADRYDFMPVAEALQESLLTTRFVLPQAPTRPVTINGGYEMPSWYDIKAVSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 19 - protein, 218 aa, Pseudomonas fluorescens
MTEPLILQPAKPADACVIWLHGLGADRYDFMPVAEALQESLLTTRFVLPQAPTRPVTINGGYEMPSWYDIKAMSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHSVLPQEIHDIGAWLAARLG

SEQ ID NO: 20 - protein, 218 aa, Pseudomonas fluorescens
MTEPLILQPAKPADACVIWLHGLGADRYDFMPVAEALQESLLTTRFVLPQAPTRPVTINGGYEMPSWYDIKAMSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHTVLPQEIHDIGAWLAARLG

SEQ ID NO: 21 - protein, 218 aa, Pseudomonas fluorescens
MTEPLILQPAKPADACVIWLHGLGADRYDFMPVAEALQESLLTTRFVLPQAPTRPVTINGGYEMSSWYDIKAHSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 22 - protein, 218 aa, Pseudomonas fluorescens
MTEPLILQPAKPADACVIWLHGLGADRYDFMPVAEALQESLLTTRFVLPQAPTRPVTINGGYDMPSWYDIKAMSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 23 - protein, 218 aa, Pseudomonas fluorescens
MTEPLILQPAKPADACVIWLHGLGGDRYDFMPVAEALQESLLTTRFVLPQAPTRPVTINGGYEMPSWYDLKAVSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 24 - protein, 218 aa, Pseudomonas fluorescens
MTEPLILQPAKPADACVIWLHGLGADRYDFMPVAEALQESLLTTRFVLPQAPTRPVTINGGYEMPSWYDLKAVSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHSVLPQEIHDIGAWLAARLG

SEQ ID NO: 25 - protein, 218 aa, Pseudomonas fluorescens
MTEPLILQPAKPADACVIWLHGLGADRYDFMPVAEALQESLLTTRFVLPQAPTRPVTINGGYEMPSWYDIKAMSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHTILPQEIHDIGAWLAARLG

SEQ ID NO: 26 - protein, 218 aa, Pseudomonas fluorescens
MTEPLILQPAKPADACVIWLHGLGADRYDFMPVAEALQESLLTTRFVLPQAPTRPVTINGGYEMPSWYDIKAVSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHSVLPQEIHDIGAWLAARLG

SEQ ID NO: 27 - protein, 218 aa, Pseudomonas fluorescens
MTEPLILQPAKPADACVIWLHGLGAGRTDFMPVAEALQESLLTTRFVLPQAPTRPVTINGGYEMPSWYDLKAVSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 28 - protein, 218 aa, Pseudomonas fluorescens
MTEPLILQPAKPADACVIWLHGLGAGRTSHRPVAEALQESLLTTRFVLPQAPTRPVTINGGYEMPSWYDIKAMSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 29 - protein, 218 aa, Pseudomonas fluorescens
MTEPLILQPAKPADACVIWLHGLGADRYDFKPVAEALQESLLTTRFVLPQAPTRPVTINGGYEMPSWYDIKAMSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 30 - protein, 218 aa, Pseudomonas fluorescens
MTEPLILQPAKPADACVIWLHGLGADRYDFRPVAEALQESLLTTRFVLPQAPTRPVTINGGYEMPSWYDIKAMSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 31 - protein, 218 aa, Pseudomonas fluorescens
MTEPLILQPAKPADACVIWLHGLGADRYDFMPVAEALQESLLTTRFVLPQAPTRPVTINGGYEMPSWYDLKAMSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 32 - protein, 218 aa, Pseudomonas fluorescens
MTEPLILQPAKPADACVIWLHGLGADRYDFMPVAEALQESLLTTRFVLPQAPTRPVTINGGYEMPSWYDIKALSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 33 - protein, 218 aa, Pseudomonas fluorescens
MTEPLILQPAKPADACVIWLHGLGADRTDFMPVAEALQESLLTTRFVLPQAPTRPVTINGGYEMPSWYDIKAVSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 34 - protein, 218 aa, Pseudomonas fluorescens
MTEPLILQPAKPADACVIWLHGLGAGRYDFMPVAEALQESLLTTRFVLPQAPTRPVTINGGYEMPSWYDIKAVSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 35 - protein, 218 aa, Pseudomonas fluorescens
MTEPLILQPAKPADACVIWLHGLGGDRTDFMPVAEALQESLLTTRFVLPQAPTRPVTINGGYEMPSWYDIKAMSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 36 - protein, 218 aa, Pseudomonas fluorescens
MTEPLILQPAKPADACVIWLHGLGADRYDFMPVAEALQESLLTTRFVLPQAPTRPVTINGGYEMPSWYDLKAASPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 37 - protein, 218 aa, Pseudomonas fluorescens
MTEPLILQPAKPADACVIWLHGLGADRYDFMPVAEALQESLLTTRFVLPQAPTRPVTINGGYEMPSWYDLKALSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 38 - protein, 218 aa, Pseudomonas fluorescens
MTEPLILQPAKPADACVIWLHGLGADRYDHMPVAEALQESLLTTRFVLPQAPTRPVTINGGYEMPSWYDIKAMSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 39 - protein, 218 aa, Pseudomonas fluorescens
MTEPLILQPAKPADACVIWLHGLGADRYDFMPVAEALQESLLTTRFVLPQAPTRPVTINGGYEMPSWYDIKAMSPARSISLEELEVSAKMVTDLIEAQKRTGIDASRIFLAGFSQGGAVVFHTAFINWQGPLGGVIAVNTYAPTFGDELELSASQQRIPALCLHGQYDDVVQNAMGRSAFEHLKSRGVTVTWQEYPMGHEVLPQEIHDIGAWLAARLG

SEQ ID NO: 40 - protein, 218 aa, Pseudomonas fluorescens
MTEPLILQPAKPADACVIWLHGLGGDRYDFMPVAEALQESLLTTRFVLPQAPTRPVTINGGYEMPSWYDIKAMSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 41 - protein, 218 aa, Pseudomonas fluorescens
MTEPLILQPAKPADACVIWLHGLGADRYDFMPVAEALQESLLTTRFVLPQAPTRPVTINGGYEMPSWYDLKAVSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 42 - protein, 218 aa, Pseudomonas fluorescens
MTEPLILQPAKPADACVIWLHGLGADRYDFMPVAEALQESLLTTRFVLPQAPTRPVTINGGYEMPSWYDLKASSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 43 - protein, 218 aa, Pseudomonas fluorescens
MTEPLILQPAKPADACVIWLHGLGAGRTSHRPVAEALQESLLTTRFVLPQAPTRPVTINGGYEMPSWYDIKAMSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHTILPQEIHDIGAWLAARLG

SEQ ID NO: 44 - protein, 236 aa, Chromohalobacter salexigens
MADSSPLIIEPRQARAADATVILLHGLGADGHDFEPLVPALPLAKDLAVRFVLPHAPRMPVTVNGGMEMPAWYDILDMNLGRRIDEAQLKASADMVHGLIDAEIARGIDSRRIIVAGFSQGGAVAYHAALTYPKPLGGLLALSTYFATATSIEPSEANRALPIEVHHGSFDPVVPEALGHEGAERAEALGYAVTYRTYPMQHALCPEQIEDIGQWLNARLGAKEAGGSLEHHHHHH

SEQ ID NO: 45 - protein, 232 aa, Halomonas sp.
MTAPGELIIEPKDGQPADACVFIIHGLGADGHDFEPLVPALALPKDSRVRFIMPHAPRLPVTINGGMVMPAWYDILAMDLGRRVDERQLKQSAERIQALIQEQIDQGIDSQRIIVAGFSQGGAVAYHAALTFPAPLGGLLAMSTYFATADNIDLAEANRQIPIEVQHGNFDPIVPESLGRSGADRLKEMGYAVNYRQYPMAHALCPQQVNDIGKWLSARLNGGSLEHHHHHH

SEQ ID NO: 46 - protein, 233 aa, Francisella tularensis
MNYELMEPAKQARFCVIWLHGLGADGHDFVDIVNYFDVSLDEIRFIFPHADIIPVTINMGMQMRAWYDIKSLDANSLNRVVDVEGINSSIAKVNKLIDSQVNQGIASENIILAGFSQGGIIATYTAITSQRKLGGIMALSTYLPAWDNFKGKITSINKGLPILVCHGTDDQVLPEVLGHDLSDKLKVSGFANEYKHYVGMQHSVCMEEIKDISNFIAKTFKIGGSLEHHHHHH

SEQ ID NO: 47 - protein, 304 aa, Sphingobium sp.
MTNDERILSWNETPSKPRYTPPPGAIDAHCHVFGPMAQFPFSPKAKYLPRDAGPDMLFALRDHLGFARNVIVQASCHGTDNAATLDAIARAQGKARGIAVVDPAIDEAELAALHEGGMRGIRFNFLKRLVDDAPKDKFLEVAGRLPAGWHVVIYFEADILEELRPFMDAIPVPIVIDHMGRPDVRQGPDGADMKAFRRLLDSREDIWFKATCPDRLDPAGPPWDDFARSVAPLVADYADRVIWGTDWPHPNMQDAIPDDGLVVDMIPRIAPTPELQHKMLVTNPMRLYWSEEMGGSLEHHHHHH

SEQ ID NO: 48 - protein, 247 aa, Pseudomonas putida
MLTEGISIQSYDGHTFGALVGSPAKAPAPVIVIAQEIFGVNAFMRETVSWLVDQGYAAVCPDLYARQAPGTALDPQDERQREQAYKLWQAFDMEAGVGDLEAAIRYARHQPYSNGKVGLVGYCLGGALAFLVAAKGYVDRAVGYYGVGLEKQLKKVPEVKHPALFHMGGQDHFVPAPSRQLITEGFGANPLLQVHWYEEAGHSFARTSSSGYVASAAALANERRLDFLAPLQSKKPGGSLEHHHHHH

SEQ ID NO: 49 - protein, 257 aa, Geobacillus stearothermophilus
MKIVPPKPFFFEAGERAVLLLHGFTGNSADVRMLGRFLESKGYTCHAPIYKGHGVPPEELVHTGPDDWWQDVMNGYEFLKNKGYEKIAVAGLSLGGVFSLKLGYTVPIEGIVTMCAPMYIKSEETMYEGVLEYAREYKKREGKSEEQIEQEMEKFKQTPMKTLKALQELIADVRDHLDLIYAPTFVVQARHDEMINPDSANIIYNEIESPVKQIKWYEQSGHVITLDQEKDQLHEDIYAFLESLDWGGSLEHHHHHH

SEQ ID NO: 50 - protein, 281 aa, unidentified
  REGION 1..281: note = Description of Unknown: Uncultured bacterium FLS12
MALFQCDFFSDVLGLSTSMTVILPQETTGQIGMAGGSERREHPTLFLLHGLSDDHTIWLRRTSIERYVAEMGLAVVMPAVHRSFYTDMAHGLQYWTFISEELPALARSFFPLATAREDTFVAGLSMGGYGALKLGMRHPERFAAAASLSGALDITFDPAEHIAMEDDVWVAEQRNIFGDLAALPGSDHDLFALAERMAQSDGPVPKLYQCCGTEDFLYEDNVRFRDHVRGLGLDFMYEESPGEHEWGYWDAQIQRVLAWLPLRPPGTAPAGGSLEHHHHHH

SEQ ID NO: 51 - protein, 259 aa, Bradyrhizobium sp.
MATETIAMDWVDIGTNGESRLAYLARPVVTGRLPAVIVMPAIHGINTYIKDVAIDLAKAGFVALLIDIHSPEQEPDLSNAEKIQIAVETLDDRKVLKDVDAAVRYLEQHAAVRADRLGILGFCVGGTYALLAARTPAIRVSVGFYGLLEYQSRTDNKPVSPLDSVAQFTAPILFHVGDKDPWIDSKMLAEFTKRMQQHQKSYELCIYRGAGHAFHEHFRDAYRPIAAQSAWNNTLIYLRWHLCGKRTVGGSLEHHHHHH

SEQ ID NO: 52 - protein, 497 aa, Myxococcus fulvus
MNGLRWRLTGVVMAWVLVAPLAEAANVKQEVDRYISGFHQKGLFNGTVLVANERGILLKKGYGAANLEWKVPNAPDTKFRIGSITKSFTATVILQLAAEGKLQLDDPITKHLPDYRKDTGDRVTITHLLNHTSGIPSYTSKPAIMKDADGFESVAAFVKKACSDDLEFEPGTKYAYNNSGYFLLGAIIEKLTGQTYAEAVQARILGPLGMKDTGYDVSATVLPKRASGYAQAPGGIVNAAWLDMNLPYAAGSLYSTVEDLYRWERAFHGDTLLPAALKQKMLTPGLAHYGFGWVMSDMTLHDGKTKLPGIFHTGGINGFSSILVRVPERKEAVILLDNMTHGGLQELAGGVLSILHGLTPRPARMPIGNVMMESLGKGSVAQAIATYRTLKKTKQAEYDFSERHLNTVGYHLLRSGRAADAIEVFKLNVEMFPEAANCHDSLGEAYAAHGDKARAITSYRKALELAPKNEHAVKMLEQLEEPAAKRGGSLEHHHHHH

SEQ ID NO: 53 - protein, 279 aa, Mesorhizobium japonicum
MSDTKVYLLDGGSLVLDGYHVFWNRGPGGEVRFPVYSILIEHAEGRFLIDTGYDYDHVMKVLPFEKPIQEKHQTIPGALGLLGLEPRDIDVVVNSHFHFDHCGGNKYFPHAKKICHRSEVPQACNPQPFEHLGYSDLSFSAEAAEARGATAQLLEGTTRANSTFEGIDGDVDLARGVKLISTPGHSIGHYSLLVEFPRRKPILFTIDAAYTQKSLETLCQAAFHIDPVAGVNSMRKVKKLAEDHGAELMYSHDMDNFKTYRTGTQFYGGGSLEHHHHHH

SEQ ID NO: 54 - protein, 310 aa, Rhizobium radiobacter
MLPADQAGIPPCQGPRARSAPISFAIPKGAWDTHLHVFGPTAVFPYAEKRPYTPPDSPLEDYLALMERLGIERGVCVHPNVHGIDNSVTIDAVERSDRRLLGIIKPHRVMTFTELRDLKTRGVRGVRFAFNPQHGSGALDTELFERMHGWCRELDWCINMHFAPDALEGLCDLIAGAETPIIIDHFGRVETAAGVNQLPFKILRDLATLDHVWIKLTGADRISHSGVPYDDVVPFAHALSEIAPDRLLWGSDWPHSGYFDPKRMPDDGDLLNLVARFAPDVALRHKILVDNPARLFGVIGGSLEHHHHHH

SEQ ID NO: 55 - protein, 393 aa, Aeromonas enteropelogenes
MKQRIALSLLALGPLLLVPRVYAAADEPMANIVEKAVQPLLEEYRIPGMAVAVLKEGKPHYFNYGVANRESGRRISERTLFEIGSVSKTFTATLGTYAVVKGGFRLDDKVSQHAPWLQNSAFDRVTMAQLATYSAGGLPLQFPDAVDSNERMRQYYRQWSPLYAAGTHREYSNPSIGLFGHLAASTLGQPFRQLMSQTLLPKLDLQHTYLEVPDAAMVDYAYGYSKEDKPVRVNPGVLADEAYGIKTSAADLIKFVGANMTGSGDKAVQQALAMTRTGFYSVGEMTQGLGWESYAYPVTEQALLAGNSPAVSFKANPVKPFVAPRVMGNERLYNKTGSTNGFGAYVVFVPARGVGIVMLANRNYPIEARVKAAYAIMRHLAPGGSLEHHHHHH

SEQ ID NO: 56 - protein, 388 aa, Escherichia coli
MFKTTLCALLITASCSTFAAPQQINDIVHRTITPLIEQQKIPGMAVAVIYQGKPYYFTWGYADIAKKQPVTQQTLFELGSVSKTFTGVLGGDAIARGEIKLSDPTTKYWPELTAKQWNGITLLHLATYTAGGLPLQVPDEVKSSSDLLRFYQNWQPAWAPGTQRLYANSSIGLFGALAVKPSGLSFEQAMQTRVFQPLKLNHTWINVPPAEEKNYAWGYREGKAVHVSPGALDAEAYGVKSTIEDMARWVQSNLKPLDINEKTLQQGIQLAQSRYWQTGDMYQGLGWEMLDWPVNPDSIINGSDNKIALAARPVKAITPPTPAVRASWVHKTGATGGFGSYVAFIPEKELGIVMLANKNYPNPARVDAAWQILNALQGGSLEHHHHHH

SEQ ID NO: 57 - protein, 347 aa, Scheffersomyces stipitis
MSKYKILDSHIHLYSLANIPLLHWDEGNPLHGNRRLDEYIENSQSTQFDVEGVVWIECDAKIDLTQGLKGLENPIEEYLYICRNINGKLLPEEGVSTPFKRRLIKAMIPFAPMPLGSAGVEEYVKALKTRNSSEFHLVKGFRYLIQDKPPLTISDPHFVSSFQWLDSNGYVFDLGIDMRSGGLWQFKETLEVFKKVPNLKYIINHLTKPCLDFDPETIDSNPDFLSWKRLVTEMYITTPNSYMKLSGGFSEVEQDVALDVTSTSRHVYPWFKVVYELWGPERTIFASNWPVCAIPAGQNLTEKWFQVCETLFDSIGMDEDTRRKIYYSNAFKAYNIGGSLEHHHHHH

SEQ ID NO: 58 - protein, 452 aa, Escherichia coli
MSEISRQEFQRRRQALVEQMQPGSAALIFAAPEVTRSADSEYPYRQNSDFWYFTGFNEPEAVLVLIKSDDTHNHSVLFNRVRDLTAEIWFGRRLGQDAAPEKLGVDRALAFSEINQQLYQLLNGLDVVYHAQGEYAYADVIVNSALEKLRKGSRQNLTAPATMIDWRPVVHEMRLFKSPEEIAVLRRAGEITAMAHTRAMEKCRPGMFEYHLEGEIHHEFNRHGARYPSYNTIVGSGENGCILHYTENECEMRDGDLVLIDAGCEYKGYAGDITRTFPVNGKFTQAQREIYDIVLESLETSLRLYRPGTSILEVTGEVVRIMVSGLVKLGILKGDVDELIAQNAHRPFFMHGLSHWLGLDVHDVGVYGQDRSRILEPGMVLTVEPGLYIAPDAEVPEQYRGIGIRIEDDIVITETGNENLTASVVKKPEEIEALMVAARKQGGSLEHHHHHH

SEQ ID NO: 59 - protein, 302 aa, Caulobacter vibrioides
MNAVTCVWDLKATLGEGPIWYDDSLWFVDIKSHKIHNYNPATDERFSFDAPEPVTFIAPLAPNARAGFVVGLKSGLHRFHPVMGGFKPLIQVESAELNNRPNDATVDHGGRLWFGTMHDDEEAKSGSLYRMDSTGVARMDKDICITNGPCVSPDGKTLYHTDTLEKIIWAYDLAEDGTLSNKRGFVNFQGENAVYPDGSVVDSEGYLWTALWGGFGVVRISPAGELVARIELPAPNVTKPCFGGPDLKTLYFTTARKGLSDETLAQYPLSGGLFGVRVDVAGQPQHEVRLVGGSLEHHHHHH

SEQ ID NO: 60 - protein, 230 aa, Pelagibacterium halotolerans
MTEPVKLSGPMLPAVSGAAKSLVVLLHGYGSDGRDLIALGQFWRDSFPDTMFVAPNAPHVCGGNPFGYEWFPLDLERDRTLARLAGAETAHPVLDAFLADLWAQTGLGPADTILVGFSQGAMMALYTGLRLPEPLKAIIAFSGLIVAPEKLEAEIASKPPVLLIHGDLDDVVPVIGSETALPKLIDLGIDARLHISQGSGHTIAQDGLDTATAFLREILGGSLEHHHHHH

SEQ ID NO: 61 - protein, 308 aa, Rhodococcus erythropolis
MSATDTARAKELLASLVSMPDATIDDFRALYEQVCATFELPDDAQVEPVDANGADALWVSAPGVSADTVAVVVHGGGFTMGSAHGYRELGYRLSKSGNLRALVVDYRLAPESPFPAPVDDVVAAYRYARSLDGVENVFLVGDSAGGGIAMSALITLRDAGEQLPDAAVVLSPLVDLAGESPSLVDRAHLDPLPAAVLVNGMGGLYLNGLDVRHPVASPMHGDLTGLPATLVLVGTDEGLHDDSTRLVDKLKAADVEVQLEIGEGLPHIWPIFSFHPDAVAATDRIGEFLRSHVAAPRGGSLEHHHHHH

SEQ ID NO: 62 - protein, 311 aa, Azospirillum brasilense
MQQIHPAGQATLLADTRNTLGEGATWCDRTRALYWVDIEGAQLWRCRADGSDLTPWPMPERLACFALTDDPDVLLVGLATHLAFFDLRSGAFTRIVEVEPELPTRLNDGRCDGSGAFVFGMKDEGAEPPRAVGGFYRLNADLTLERLALPPAAIANSIGFSPDGSKMYFCDSLVREIFVCDYRPGGEVANVRPFARLTDPDGDPDGSIVDRDGGLWNAQWGGRRVVRYGPDGVETDRVAVPTAQPSCTALDGEGRLYVTSARVGLSDDALADDPHAGGVFVAQTRHAGMATARFAGTPRGGGSLEHHHHHH

SEQ ID NO: 63 - protein, 1088 aa, Acetivibrio thermocellus
MKNKRVLAKITALVVLLGVFFVLPSNISQLYADYEVVHDTFEVNFDGWCNLGVDTYLTAVENEGNNGTRGMMVINRSSASDGAYSEKGFYLDGGVEYKYSVFVKHNGTGTETFKLSVSYLDSETEEENKEVIATKDVVAGEWTEISAKYKAPKTAVNITLSITTDSTVDFIFDDVTITRKGMAEANTVYAANAVLKDMYANYFRVGSVLNSGTVNNSSIKALILREFNSITCENEMKPDATLVQSGSTNTNIRVSLNRAASILNFCAQNNIAVRGHTLVWHSQTPQWFFKDNFQDNGNWVSQSVMDQRLESYIKNMFAEIQRQYPSLNLYAYDVVNEAVSDDANRTRYYGGAREPGYGNGRSPWVQIYGDNKFIEKAFTYARKYAPANCKLYYNDYNEYWDHKRDCIASICANLYNKGLLDGVGMQSHINADMNGFSGIQNYKAALQKYINIGCDVQITELDISTENGKFSLQQQADKYKAVFQAAVDINRTSSKGKVTAVCVWGPNDANTWLGSQNAPLLFNANNQPKPAYNAVASIIPQSEWGDGNNPAGGGGGGKPEEPDANGYYYHDTFEGSVGQWTARGPAEVLLSGRTAYKGSESLLVRNRTAAWNGAQRALNPRTFVPGNTYCFSVVASFIEGASSTTFCMKLQYVDGSGTQRYDTIDMKTVGPNQWVHLYNPQYRIPSDATDMYVYVETADDTINFYIDEAIGAVAGTVIEGPAPQPTQPPVLLGDVNGDGTINSTDLTMLKRSVLRAITLTDDAKARADVDKNGSINSTDVLLLSRYLLRVIDKFPVAENPSSSFKYESAVQYRPAPDSYLNPCPQAGRIVKETYTGINGTKSLNVYLPYGYDPNKKYNIFYLMHGGGENENTIFSNDVKLQNILDHAIMNGELEPLIVVTPTFNGGNCTAQNFYQEFRQNVIPFVESKYSTYAESTTPQGIAASRMHRGFGGFSMGGLTTWYVMVNCLDYVAYFMPLSGDYWYGNSPQDKANSIAEAINRSGLSKREYFVFAATGSDHIAYANMNPQIEAMKALPHFDYTSDFSKGNFYFLVAPGATHWWGYVRHYIYDALPYFFHEGGSLEHHHHHH

SEQ ID NO: 64 - protein, 316 aa, Xanthomonas campestris
MDSHCRVRPAGPAVPADCDPPRITHAALAARLGDARLLTLYDQATWSEGPAWWEAQRTLVWSDLVGRRVLGWREDGTVDVLLDATAFTNGNAVDAQQRLVHCEHGRRAITRSDADGQAHLLVGRYAGKRLNSPNDLIVARDGAIWFTDPPFGLRKPSQGCPADPELAHHSVYRLPPDGSPLQRMADLDHPNGLAFSPDEQTLYVSQTPEQGHGSVEITAFAWRDGALHDRRHFASVPDGLPDGFCVDRGGWLWSSSGTGVCVFDSDGQLLGHIPTPGTASNCTFDQAQQRLFITGGPCLWMLPLPGGSLEHHHHHH

SEQ ID NO: 65 - protein, 292 aa, Aspergillus niger
MKQFSAKYALILLATAGQALAASTQGISEDLYNRLVEMATISQAAYADLCNIPSTIIKGEKIYNAQTDINGWILRDDTSKEIITVFRGTGSDTNLQLDTNYTLTPFDTLPQCNDCEVHGGYYIGWISVQDQVESLVKQQASQYPDYALTVTGHSLGASMAALTAAQLSATYDNVRLYTFGEPRSGNQAFASYMNDAFQVSSPETTQYFRVTHSNDGIPNLPPADEGYAHGGVEYWSVDPYSAQNTFVCTGDEVQCCEAQGGQGVNDAHTTYFGMTSGACTWGGSLEHHHHHH

SEQ ID NO: 66 - protein, 412 aa, Gibberella intermedia
MPSSISVLAAGILVPVLGAVAAKLPPTAQIIDQKSFNVLKDVPPPAVANDSLVFTWPGVTEESLVEKPFHVYDEEFYDVIGKDPSLTLIATSDTDPIFHEAVVWYPPTEEVFFVQNAGAPAAGTGLNKSSIIQKISLKEADEVRKGKKDEVKVTVVDSNPQVINPNGGTYYKGNIIFAGEGQGDDVPSALYLMNPLPPYNTTTLLNNYFGRQFNSLNDVGINPRNGDLYFTDTLYGYLQDFRPVPGLRNQVYRYNFDTGAVTVVADDFTLPNGIGFGPDGKKVYVTDTGIALGFYGRNLSSPASVYSFDVNQDGTLQNRKTFAYVASFIPDGVHTDSKGRVYAGCGDGVHVWNPSGKLIGKIYTGTVAANFQFAGKGRMIITGQTKLFYVTLGASGPKLYDGGSLEHHHHHH

SEQ ID NO: 67 - protein, 318 aa, Euglena gracilis
MRTLATVASQTDAWTGEGPVWCAARRCLYYVDLGDTRPGKLHVYHPERCVEEIHDLPAMTKDFTQVTAVTVVQNEPHRLAVATEAGVFLYDCQSGDLRRLTGELQPELPKGSYRSNDGKCDPRGRFLIGTMLFSADAPSGGLFSVAGSTIQQLLTGVTIGNGLAWSANGRTMYFIDSPLKRIDAFEYHLDAGTLGARRTAFDFADYFAQQAGWEEAAPDGMTIDAEGLLWVAIYGGGAALRVDPAKEEVVCRVDCPAKYTTSVALGGPARDTLYITSFRRGDAGPDAGAVFQCRAPAPGPPPAEFRLGGSLEHHHHHH

SEQ ID NO: 68 - protein, 277 aa, Pseudomonas putida
MFKLLSKLLVYLTASIMAIASPLAFSVDSSGEYPTVSEIPVGEVRLYQIADGVWSHIATRSFDGAVYPSNGLIVRDGDELLLIDTAWGAKNTAALLAEIEKQIGLPVTRAVSTHFHDDRVGGVDVLRAAGVATYASPSTRRLAEVEGSEIPTHSLEGLSSSGDAVRFGPVELFYPGAAHSTDNLVVYVPSASVLYGGCAIYELSRTSAGNVADADLAEWPTSIERIQQHYPEAQFVIPGHGLPGGLDLLKHTTNVVKAHTNRSVVEGGSLEHHHHHH

SEQ ID NO: 69 - protein, 277 aa, Pseudomonas aeruginosa
MLKVISSLLVYMTASVMAVASPLAHSGEPSGEYPTVNEIPVGEVRLYQIADGVWSHIATQSFDGAVYPSNGLIVRDGDELLLIDTAWGAKNTAALLAEIEKQIGLPVTRAVSTHFHDDRVGGVDVLRAAGVATYASPSTRRLAEAEGNEIPTHSLEGLSSSGDAVRFGPVELFYPGAAHSTDNLVVYVPSANVLYGGCAVHELSRTSAGNVADADLAEWPTSVERIQKHYPEAEVVIPGHGLPGGLDLLQHTANVVKAHKNRSVAEGGSLEHHHHHH

SEQ ID NO: 70 - protein, 585 aa, Rhodococcus sp.
MVDGNYSVASNVMVPMRDGVRLAVDLYRPDADGPVPVLLVRNPYDKFDVFAWSTQSTNWLEFVRDGYAVVIQDTRGLFASEGEFVPHVDDEADAEDTLSWILEQAWCDGNVGMFGVSYLGVTQWQAAVSGVGGLKAIAPSMASADLYRAPWYGPGGALSVEALLGWSALIGTGLITSRSDARPEDAADFVQLAAILNDVAGAASVTPLAEQPLLGRLIPWVIDQVVDHPDNDESWQSISLFERLGGLATPALITAGWYDGFVGESLRTFVAVKDNADARLVVGPWSHSNLTGRNADRKFGIAATYPIQEATTMHKAFFDRHLRGETDALAGVPKVRLFVMGIDEWRDETDWPLPDTAYTPFYLGGSGAANTSTGGGTLSTSISGTESADTYLYDPADPVPSLGGTLLFHNGDNGPADQRPIHDRDDVLCYSTEVLTDPVEVTGTVSARLFVSSSAVDTDFTAKLVDVFPDGRAIALCDGIVRMRYRETLVNPTLIEAGEIYEVAIDMLATSNVFLPGHRIMVQVSSSNFPKYDRNSNTGGVIAREQLEEMCTAVNRIHRGPEHPSHIVLPIIKRGGSLEHHHHHH

SEQ ID NO: 71 - protein, 277 aa, Trypanosoma brucei
MSFKPTISVHATPQELSAAGCRKIVEIIEASGSQQWPLSIALAGGSTPKMTYARLHDEHLNLLREKRALRFFMGDERMVPADSTDSNYNMAREVLLHDIPDDLVFPFDTSAVTPSAEATSADAMRVAEAYGKQLASLLPLKSVGEAGPKVPVFDVVLLGLGSDGHTASIFPGSQAEKETDGKVVVSVGFPSETMKPKVWRVTLSPATIMQARNVIVLATGAEKKWVVDGILADTAHKAPVARFLRGCEGNVSFLLDKEIAENLAKFGGSLEHHHHHH

SEQ ID NO: 72 - protein, 274 aa, Paraburkholderia xenovorans
MPYAAVNGTELHYRIDGERHGNAPWIVLSNSLGTDLSMWAPQVAALSKHFRVLRYDTRGHGHSEAPKGPYTIEQLTGDVLGLMDTLKIARANFCGLSMGGLTGVALAARHADRIERVALCNTAARIGSPEVWVPRAVKARTEGMHALADAVLPRWFTADYMEREPVVLAMIRDVFVHTDKEGYASNCEAIDAADLRPEAPGIKVPALVISGTHDLAATPAQGRELAQAIAGARYVELDASHISNIERADAFTKTVVDFLTEQKGGSLEHHHHHH

SEQ ID NO: 73 - protein, 286 aa, Thermogutta terrifontis
MAQRVKITTTATPGEIELAFEDTGTGLPVLLVHGFPLDRTMWKAQREELCDEFRVIVPDLRGFGESQVIPGVATMEAMADDLAGLCNHLGLTGKIVLGGLSMGGYVAFAFARKYRDRLAGLILCDTRARPDSPEAKENRRRVAERVRREGPGFIAEEMIPRLCCESTFRNHPEVIEKIRQMILSAPPEGVAAAALGMAERPDSTDLLPALSCPTLVLVGQFDAISPPEEMEAMARTIPQSQFVVIPDAGHLPPMEQPERVTQAIREWLRKVHTEAGGSLEHHHHHH

SEQ ID NO: 74 - protein, 309 aa, Streptomyces purpurascens
MSERIVPSGDVELWSDDFGDPADPALLLVMGGNLSALGWPDEFARRLADGGLHVIRYDHRDTGRSTTRDFAAHPYGFGELAADAVAVLDGWGVDRAHVVGLSMGATITQVIALDHHDRLSSLTMLLGGGLDIDFDANIERVMRGEPTLDGLPGPQQPFLDALALMNQPAEGRAAEVAKRVSKWRILSGTGVPFDDAEYARWEERAIDHAGGVLAEPYAHYSLTLPPPSRAAELREVTVPTLVIQAEHDPIAPAPHGKHLAGLIPTARLAEIPGMGHALPSSVHGPLAEVILAHTRSAAGGSLEHHHHHH

SEQ ID NO: 75 - protein, 261 aa, Bacillus thuringiensis
MTVKKLYFIPAGRCMLDHSSVNSALTPGKLLNLPVWCYLLETEEGPILVDTGMPESAVNNEGLFNGTFVEGQILPKMTEEDRIVNILKRVGYEPDDLLYIISSHLHFDHAGGNGAFTNTPIIVQRTEYEAALHREEYMKECILPHLNYKIIEGDYEVVPGVQLLYTPGHSPGHQSLFIETEQSGSVLLTIDASYTKENFEDEVPFAGFDPELALSSIKRLKEVVKKEKPIIFFGHDIEQEKSCRVFPEYIGGSLEHHHHHH

SEQ ID NO: 76 - protein, 377 aa, Dickeya dadantii
MLKTISGTLALSLIIAASVHQAQAATTYNAVVSKSSSDGKTFKTIADAIASAPAGSTPFVILIKNGVYNERLTITRNNLHLKGESRNGAVIAAATAAGTLKSDGSKWGTAGSSTITISAKDFSAQSLTIRNDFDFPANQAKSDSDSSKIKDTQAVALYVTKSGDRAYFKDVSLVGYQDTLYVSGGRSFFSDCRISGTVDFIFGDGTALFNNCDLVSRYRADVKSGNVSGYLTAPSTNINQKYGLVITNSRVIRESDSVPAKSYGLGRPWHPTTTFSDGRYADPNAIGQTVFLNTSMDNHIYGWDKMSGKDKNGNTIWFNPEDSRFFEYKSYGAGATVSKDRRQLTDAQAAEYTQSKVLGDWTPTLPGGSLEHHHHHH

SEQ ID NO: 77 - protein, 322 aa, Archaeoglobus fulgidus
MLDMPIDPVYYQLAEYFDSLPKFDQFSSAREYREAINRIYEERNRQLSQHERVERVEDRTIKGRNGDIRVRVYQQKPDSPVLVYYHGGGFVICSIESHDALCRRIARLSNSTVVSVDYRLAPEHKFPAAVYDCYDATKWVAENAEELRIDPSKIFVGGDSAGGNLAAAVSIMARDSGEDFIKHQILIYPVVNFVAPTPSLLEFGEGLWILDQKIMSWFSEQYFSREEDKFNPLASVIFADLENLPPALIITAEYDPLRDEGEVFGQMLRRAGVEASIVRYRGVLHGFINYYPVLKAARDAINQIAALLVFDGGSLEHHHHHH

SEQ ID NO: 78 - protein, 308 aa, Bacillus thuringiensis
MTLDLQVQSFLAQGGNLNTLTGEEHGEAKAVFKVEDFYIPVKDGEIKLRVYTPNEKESLPVFVYLHGGGWVAGDIQAVDTLCQNIAHDAECIVVAVEYRLAPTHKFPVPLEDCYEATKWVAENSSMLNADKTKIAIGGDSAGGNIAAAVVIMAQKFNNLSLVAQVLVYPVVDLTLTFKAQSYRDNAEGYLLTTEGVFWATQMYLRDEIDRYNVFASPSVNNELENLPPALIITAEYDPLRDDGAAYAKRLEAAGIPVEYKCYEGMVHGFFWMAGIMDKGLQARLQVSNYLKSVFVGKGGSLEHHHHHH

SEQ ID NO: 79 - protein, 321 aa, Alicyclobacillus acidocaldarius
MPLDPVIQQVLDQLNRMPAPDYKHLSAQQFRSQQSLFPPVKKEPVAEVREFDMDLPGRTLKVRMYRPEGVEPPYPALVYYHGGGWVVGDLETHDPVCRVLAKDGRAVVFSVDYRLAPEHKFPAAVEDAYDALQWIAERAADFHLDPARIAVGGDSAGGNLAAVTSILAKERGGPALAFQLLIYPSTGYDPAHPPASIEENAEGYLLTGGMMLWFRDQYLNSLEELTHPWFSPVLYPDLSGLPPAYIATAQYDPLRDVGKLYAEALNKAGVKVEIENFEDLIHGFAQFYSLSPGATKALVRIAEKLRDALAGGSLEHHHHHH

SEQ ID NO: 80 - protein, 317 aa, Saccharolobus solfataricus
MPLDPEVRNFLQVYYKANIIDFTKYQFQEIRQKVNELLAKAVPKDPVGETRDMKIKLEDYELPIRIYSPIKRTNNGLVMHFHGGAWILGSIETEDAISRILSNSCECTVISVDYRLAPEYKFPTAVYDCFNAIVWARDNAGELGIDKDKIATFGISAGGNLVAATSLLARDNKLKLTAQVPVVPFVYLDLASKSMNRYRKGYFLDINLPVDYGVKMYIRDEKDLYNPLFSPLIAEDLSNLPQAIVVTAEYDPLRDQGEAYAYRLMESGVPTLSFRVNGNVHAFLGSPRTSRQVTVMIGALLKDIFKGSSLEHHHHHH

SEQ ID NO: 81 - protein, 317 aa, Sulfolobus islandicus
MMPLDPRIKELLESGFIVPIGKASVDEVRKIFRQLASAAPKVEVGKVEDIKIPGSEANINARVYLPKANGPYGVLIYLHGGGFVIGDVESYDPLCRAITNACNCVVVSVDYRLAPEYKFPSAVIDSFDATNWVYNNLDKFDGKMGVAIAGDSAGGNLAAVVALLSKGKLNLKYQILIYPAVGFDSVSRSMIEYSDGFFLTREHIEWFGSQYLRSPADLLDFRFSPILAQDLSGLPPALIITAEYDPLRDQGEAYANRLLQAGVPVTSVRFNNVIHGFLSFFPLIEQGRDAISLIGSVLRRTFYDKSGGSLEHHHHHH

SEQ ID NO: 82 - protein, 314 aa, Sulfurisphaera tokodaii
MIDPKIKKLLESTIQLPIGKASVEEIRSLFKQFSSLTPREEVGKIEDITIPGSETNIKARVYYPKTQGPYGVLVYYHGGGFVLGDIESYDPLCRAITNSCQCVTISVDYRLAPENKFPAAVVDSFDALKWVYNNSEKFNGKYGIAVGGDSAGGNLAAVTAILSKKENIKLKYQVLIYPAVSFDLITKSLYDNGEGFFLTREHIDWFGQQYLRSFADLLDFRFSPILADLNDLPPALIITAEHDPLRDQGEAYANKLLQSGVQVTSVRFNNVIHGFVSFFPFIEQGRDAIGLIGYVLRKVFYGKGGSLEHHHHHH

SEQ ID NO: 83 - protein, 233 aa, Ventosimonas gracilis
MTEPLIIEPSQPADSAVIWLHGLGADRFDFEPVARLLGQHLPSTRFILPQAPTRPVTFNMGHAMPSWYDILALDGSERAINPADLEASSETLIALINAQQQSGIDSKRIVLAGFSQGGAVVLHTALLRFDEKLAGVLALSTYAPTFNAETQFAESKQNLPVLCMHGSEDAVLPISMGRAVYDKLSEQGIKANWRDYPMGHEVRPEQLRDILDWLKNTLPSLPGGSLEHHHHHH

SEQ ID NO: 84 - protein, 230 aa, Ignatzschineria indica
MDKPIILDPKQSADSAVIWLHGLGATKEDFLPVAQILQRDALPHTRFILPQAPVRPVTLNNGFPMPSWYDIIALTSPREIKLSELDESSQSIIALIEAEIEKGIPLERIILAGFSQGGAVVLHTAFIAYPKNVGGVMALSTYSATFDEAITLDEKKKQIPTLHLHGSLDPVVKIELGRAAEQFLKAQGIDTRWHDYPMQHEVINDELQDIAKWLIERLGGGSLEHHHHHH

SEQ ID NO: 85 - protein, 230 aa, Pseudomonas furukawaii
MSDTLILEPTHRADACVIWLHGLGADRYDFLPVAEALQDVLGTTRFVLPQAPTRAVTINGGWAMPSWYDILAMSPERAIDEAQLEASAQQVMALAQAQVDGGIEPRRIFLAGFSQGGAVVLHTAFLRWEDELGGVLALSTYGPTFTDGMTLPDAKRQLPVLCLHGTLDDVVLPAMGRAAHDRLAAAGVPVGWRDYPMAHEVLPQQVRDIGAWLVERLHSGGSLEHHHHHH

SEQ ID NO: 86 - protein, 228 aa, Pseudomonas citronellolis
MSQPLLLEPTQPADSCVIWLHGLGADRYDFEPVARMLQKVLPRTRFILPQAPTRPVTVFNGMPAPSWYDIKAMAPARAIDEAQLDASADAVIALIEGQLAEGIAQRRIVLAGFSQGGAVVLHTGYLRWPGELGGVMALSTYGPTFDDDLQLPEAKKQQPALCLHGTYDDVVAPAMGRAAYDFLQRQGVAVQWRDYPMAHEVSNQEIADIAAWLRERLGGSLEHHHHHH

SEQ ID NO: 87 - protein, 226 aa, Pseudomonas aeruginosa
MSEPLILDAPNADACIIWLHGLGADRTDFKPVAEALQMVLPSTRFILPQAPSQAVTVNGGWVMPSWYDILAFSPARAIDEDQLNASADQVIALIDEQRAKGIAAERIILAGFSQGGAVVLHTAFRRYAQPLGGVLALSTYAPTFDDLALDERHKRIPVLHLHGSQDDVVDPALGRAAHDALQAQGVEVGWHDYPMGHEVSLEEIHDIGAWLRKRLGGSLEHHHHHH

SEQ ID NO: 88 - protein, 229 aa, Pseudomonas saudiphocaensis
MTDPLIIEPAQTADSCVIWLHGLGADRYDFQPVAEMLQQRLLHTRFVLPQAPTRAVTINGGWAMPSWYDIQAMSPARAIDQAQLEQSAQTVIELIEQQRDSGIDPRRIFLAGFSQGGAVVYHTAFLRWAGPLGGVLALSTYAPTFGDDLKLSPLQAGLPVLCLHGSRDDVVPPAMGRAAHDCLQQNQVQTQWKEYPMAHEVQPTEIQDIGDWLASRLGGGSLEHHHHHH

SEQ ID NO: 89 - protein, 229 aa, Pseudomonas fluorescens
MTEPLILQPAKPADACVIWLHGLGADRYDFMPVAEALQESLLTTRFVLPQAPTRPVTINGGYEMPSWYDIKAMSPARSISLEELEVSAKMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLGGGSLEHHHHHH

SEQ ID NO: 90 - protein, 11 aa, synthetic construct
  REGION 1..11: note = Hexahistidine tag
GGSLEHHHHHH

SEQ ID NO: 91 - protein, 6 aa, synthetic construct
  REGION 1..6: note = Description of Artificial Sequence: Synthetic 6xHis tag
HHHHHH

(19) United States
(12) Patent Application Publication
(10) Pub. No.: US 2024/0084345 A1
(43) Pub. Date: Mar. 14, 2024

(54) ENZYMATIC TREATMENT OF ANTHOCYANINS

(71) Applicants: MARS INCORPORATED, McLean, VA (US); THE REGENTS OF THE UNIVERSITY OF CALIFORNIA, Oakland, CA (US)

(72) Inventors: Thomas M. Collins, Chicago, IL (US); Rebecca J. Robbins, Chicago, IL (US); Randall Powers, Chicago, IL (US); Justin Siegel, Oakland, CA (US); Pamela Ruth Denish, Oakland, CA (US); Kathryn Gwendolyn Guggenheim, Oakland, CA (US)

(73) Assignees: MARS, INCORPORATED, McLean, VA (US); THE REGENTS OF THE UNIVERSITY OF CALIFORNIA, Oakland, CA (US)

(21) Appl. No.: 18/356,948

(22) Filed: Jul. 21, 2023

Related U.S. Application Data
(62) Division of application No. 17/711,259, filed on Apr. 1, 2022, now Pat. No. 11,753,664.
Application No. 17/711,259 is a continuation of application No. PCT/US2020/053849, filed on Oct. 1, 2020.
(60) Provisional application No. 62/909,106, filed on Oct. 1, 2019.

(51) Int. Cl. C12P 17/16; A23L 5/43; C12N 9/18; C12P 19/60

(52) U.S. Cl. CPC C12P 17/162; A23L 5/43; C12N 9/18; C12P 19/60; C12Y 301/01001; A23V 2002/00

(57) ABSTRACT

Methods for converting mixtures of anthocyanins occurring in fruit or vegetable juice or extract into particular anthocyanin molecules having desirable colorant properties are provided herein. The method of the present disclosure can be employed to increase the amount of particular anthocyanin molecules, while lowering the total number of anthocyanin molecules present in the natural juice and/or extract. The disclosure is also directed to anthocyanin molecules prepared by the methods of present disclosure and to enzymes capable of catalyzing reactions that provide such effects.

### CROSS REFERENCE TO RELATED APPLICATIONS

This application is a divisional of U.S. patent application Ser. No. 17/711,259 filed on Apr. 1, 2022, now allowed, which is a continuation of International Patent Application No. PCT/US2020/053849 filed on Oct. 1, 2020, which claims priority to U.S. Patent Application Ser. No. 62/909,106 filed on Oct. 1, 2019, all of which are incorporated herein by reference in their entireties.

### SEQUENCE LISTING

The instant application contains a Sequence Listing which has been submitted electronically in XML format and is hereby incorporated by reference in its entirety. Said XML copy, created on Jul. 21, 2023, is named 069269.0640_SL.XML and is 105,636 bytes in size.

### FIELD OF THE INVENTION

The presently disclosed subject matter relates to the use of natural and modified enzymes to convert mixtures of anthocyanins occurring in fruit or vegetable juice or extract into an anthocyanin molecule having desirable colorant properties. The presently disclosed subject matter further relates to isolation of the anthocyanin molecule having desirable colorant properties and use thereof in various applications including edible products.

### BACKGROUND OF THE INVENTION

The use and demand of alternatives for synthetic food colorants has been increasing due to consumer demand. However, natural food colorants have not been able to achieve the same color characteristics as their synthetic counterparts, for example, FD&C Blue No. 1. The lack of a suitable natural blue colorant has also made the development of a desirable natural green hue, from a combination of natural blue and yellow colorants, difficult.

Anthocyanins are known as natural food colorants responsible for reds and blues of fruits and vegetables. It is known in the art that anthocyanin-containing juices and extracts generally exhibit red hues at low pH with a hue shifts towards purple as the pH increases. For example, it is generally observed that the hue shifts from pink to purple when hydrogen is replaced with a hydroxyl group. International Patent Publication No. WO 2014/152417 discloses the isolation of fractions of anthocyanin molecules from vegetable and fruit extracts, including red cabbage, at select pHs to provide different color characteristics than those provided by the source vegetable or fruit.

The substitution pattern of anthocyanins also affects color. For example, the number of glycosyl (sugar) units and the number and type of acyl units are observed to affect color. However, these phenomena are not well understood or predictable.

Intermolecular and intramolecular interactions also affect anthocyanin color. The same anthocyanin may produce different hues depending on what other molecules are present. For example, it is believed that acyl groups on the anthocyanin sugars can fold in and protect the flavylium cation C-2 position from nucleophilic attack. Therefore, this intramolecular interaction prevents formation of the colorless carbinol pseudo-base structure. Similarly, it is believed that anthocyanin molecules self-associate, which is evidenced by the fact that a two-fold increase in anthocyanin concentration can cause a 300-fold increase in chroma and can change the hue and value as well. It is hypothesized that this self-association is similar to intramolecular stacking and prevents nucleophilic attack and formation of the carbinol pseudo-base structure.

Separation of a single compound from a complex mixture of compounds is often technically challenging and often requires a large volume of starting material. To date, the currently available blue colorant compositions that are produced from natural sources have not been satisfactory for commercial use in food products, either because of their color properties, or due to the high cost of their production. Additionally, the amount of fruit or vegetable juice or extract needed to isolate a single anthocyanin of interest has proven to be impractical. Therefore, there remains a need for natural blue anthocyanin colorants that provide similar color characteristics as synthetic counterparts that can be produced in large quantities.

### SUMMARY OF THE INVENTION

The presently disclosed subject matter relates to compositions natural and modified enzymes and their related use for catalyzing the hydrolysis of an ester in an anthocyanin compound. The anthocyanin may occur in a solution, e.g., a fruit or vegetable juice or extract, and the catalysis can produce a compound (e.g., Compound I) having desirable colorant properties.

In one aspect, the present disclosure provides a method for selectively converting one or more diacylated anthocyanins into a monoacylated anthocyanin product, the method including: a) providing a fruit or vegetable juice or extract including a diacylated anthocyanin or a mixture of anthocyanins, wherein the mixture of anthocyanins includes one or more diacylated anthocyanins and, optionally, one or more monoacylated anthocyanins; and b) subjecting the fruit or vegetable juice or extract to an enzymatic treatment by one or more enzymes, wherein the one or more enzymes selectively hydrolyzes the one or more diacylated anthocyanins into the monoacylated anthocyanin product.

In some embodiments, the fruit or vegetable juice or extract is red cabbage juice or extract. In some embodiments, the monoacylated anthocyanin product is Compound I having the following structure:

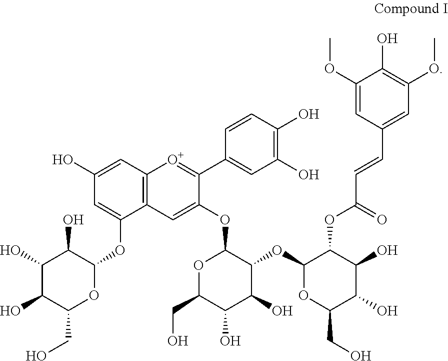 [SMILES: COc1cc(/C=C/C(=O)O[C@H]2[C@H](O[C@H]3[C@H](Oc4cc5c(O[C@@H]6O[C@H](CO)[C@@H](O)[C@H](O)[C@H]6O)cc(O)cc5[o+]c4-c4ccc(O)c(O)c4)O[C@H](CO)[C@@H](O)[C@@H]3O)O[C@H](CO)[C@@H](O)[C@@H]2O)cc(OC)c1O]

In some embodiments, the mixture of anthocyanins comprises the one or more monoacylated anthocyanins, and the enzymatic treatment additionally converts the one or more monoacylated anthocyanins into a nonacylated anthocyanin product. In some embodiments, the nonacylated anthocyanin product is Compound II having the following structure:

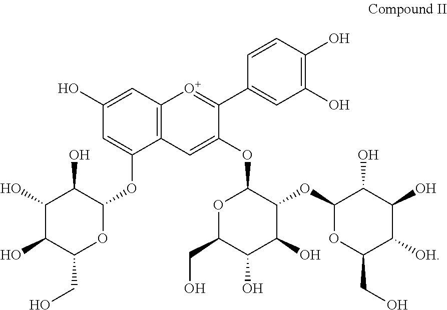 [SMILES: OC[C@H]1O[C@@H](Oc2cc(O)cc3[o+]c(-c4ccc(O)c(O)c4)c(O[C@@H]4O[C@H](CO)[C@@H](O)[C@H](O)[C@H]4O[C@@H]4O[C@H](CO)[C@@H](O)[C@H](O)[C@H]4O)cc23)[C@H](O)[C@@H](O)[C@@H]1O]

In further embodiments, the one or more enzymes comprises one or more esterases. The one or more esterases can be a naturally occurring esterase or a modified esterase.

In some embodiments, the naturally occurring esterase includes the amino acid sequence of any one of SEQ ID NO: 1-15. In some embodiments, the modified esterase is a modified carboxylesterase from Pseudomonas fluorescens. In some embodiments, the modified carboxylesterase from P. fluorescens includes one or more amino acid substitutions at position number 25, 28, 29, 30, 31, 38, 39, 63, 65, 70, 73, 200, or 201 relative to a naturally occurring carboxylesterase from P. fluorescens (e.g., the naturally occurring carboxylesterase from P. fluorescens of SEQ ID NO: 2).

In further embodiments, the one or more modified esterase is a modified carboxylesterase from Chromohalobacter salexigens. In some embodiments, the modified carboxylesterase from C. salexigens includes one or more amino acid substitutions at amino acid position number 23, 28, 75, 115, 117, 119, 121, 122, 125, 129, 168, 171, 173, 202, 209, or 212 relative to a naturally occurring carboxylesterase from C. salexigens (e.g., the naturally occurring carboxylesterase from C. salexigens of SEQ ID NO: 1).

In some embodiments, the modified esterase includes the amino acid sequence of any one of SEQ ID NO: 16-43.

In some embodiments, the monoacylated anthocyanin product is Compound I, and Compound I is isolated from the solution following the enzymatic treatment. In some embodiments, Compound I is isolated by a purification process including one or more of enzyme precipitation, solid-phase extraction, and preparatory high performance liquid chromatography (HPLC). In some embodiments, Compound I is isolated by a purification process including sequentially performing the steps of: (i) enzyme precipitation; (ii) solid-phase extraction; and, optionally, (iii) preparatory HPLC, wherein the preparatory HPLC of step (iii) is performed if the solution includes two or more anthocyanins.

In another aspect, the present disclosure features a method of producing Compound I:

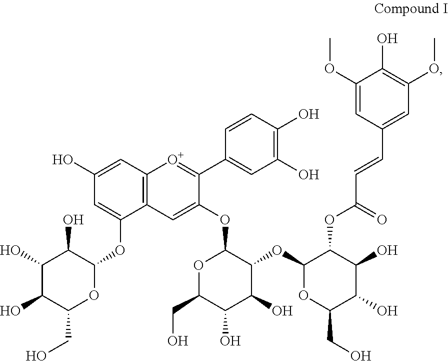 [SMILES: COc1cc(/C=C/C(=O)O[C@H]2[C@H](O[C@H]3[C@H](Oc4cc5c(O[C@@H]6O[C@H](CO)[C@@H](O)[C@H](O)[C@H]6O)cc(O)cc5[o+]c4-c4ccc(O)c(O)c4)O[C@H](CO)[C@@H](O)[C@@H]3O)O[C@H](CO)[C@@H](O)[C@@H]2O)cc(OC)c1O]

the method including contacting a solution including an anthocyanin with an enzyme, wherein the enzyme selectively hydrolyzes the anthocyanin to produce Compound I.

In some embodiments, the enzyme includes the amino acid sequence of any one of SEQ ID NO: 1-43. In some embodiments, the solution is contacted with two or more enzymes including the amino acid sequence of any one of SEQ ID NO: 1-43. In some embodiments, the solution is contacted with three or more enzymes including the amino acid sequence of any one of SEQ ID NO: 1-43. In further embodiments, the solution is contacted with four or more enzymes including the amino acid sequence of any one of SEQ ID NO: 1-43.

In some embodiments, the anthocyanin has the structure of Formula 1:

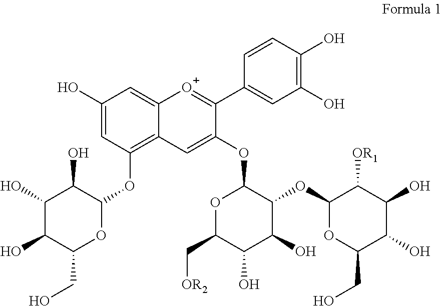

wherein R₁ is H or an acyl group and R₂ is an acyl group that is the same or different from the acyl group of R₁. In certain embodiments, the enzyme selectively hydrolyzes the anthocyanin to remove the acyl group of R₂. In some embodiments, the acyl group of R₁ and/or R₂ can be described by the structure

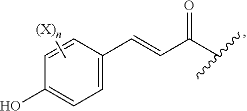

wherein X is a C₁₋₄ alkoxy group, and n is an integer from 0-2.

In further embodiments, the solution is a fruit or vegetable juice or extract, e.g., red cabbage juice or extract. In some embodiments, Compound I is present in an amount of from about 40% to about 100% by weight of a total anthocyanin content of the solution at least about 24 hours after the solution is contacted with the enzyme.

In another aspect, the present disclosure features a method of selectively hydrolyzing an anthocyanin of Formula 1:

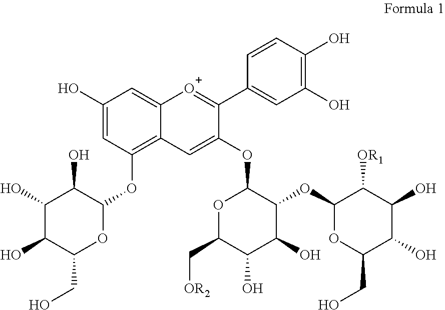

wherein R₁ is H or an acyl group, and R₂ is an acyl group that is the same or different from the acyl group of R₁; the method including contacting a solution including the anthocyanin of Formula 1 with an enzyme, wherein the enzyme selectively hydrolyzes the anthocyanin to remove the acyl group of R₂ and does not remove R₁. In some embodiments, the acyl group of R₁ and/or R₂ can be described by the structure

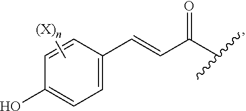

wherein X is a C₁₋₄ alkoxy group, and n is an integer from 0-2.

In some embodiments, the enzyme includes the amino acid sequence of any one of SEQ ID NO: 1-43. In some embodiments, the solution is contacted with two or more enzymes including the amino acid sequence of any one of SEQ ID NO: 1-43. In some embodiments, the solution is contacted with three or more enzymes including the amino acid sequence of any one of SEQ ID NO: 1-43. In further embodiments, the solution is contacted with four or more enzymes including the amino acid sequence of any one of SEQ ID NO: 1-43.

In some embodiments, the solution is a fruit or vegetable juice or extract. In some embodiments, the solution is red cabbage juice or extract.

In further embodiments, the selective hydrolysis produces Compound I and/or Compound II. In some embodiments, Compound I is present in an amount of from about 40% to about 100% by weight of a total anthocyanin content of the solution at least about 24 hours after the solution is contacted with the enzyme.

In some embodiments of any of the preceding methods, Compound I is isolated from the solution following the selective hydrolysis. In some embodiments, Compound I is isolated by a purification process including one or more of enzyme precipitation, solid-phase extraction, and preparatory HPLC. In certain embodiments, Compound I is isolated by a purification process including sequentially performing the steps of: (i) enzyme precipitation; (ii) solid-phase extraction; and, optionally, (iii) preparatory HPLC, wherein the preparatory HPLC of step (iii) is performed if the solution includes two or more anthocyanins.

In another aspect, the present disclosure features an edible colorant composition including: (a) a compound having the structure:

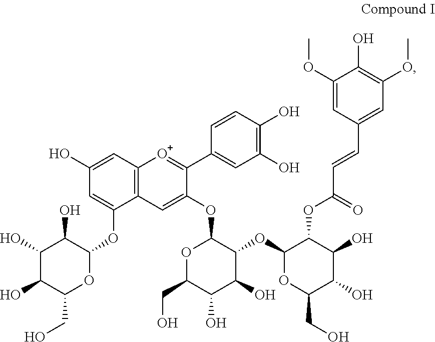 [SMILES: COc1cc(/C=C/C(=O)O[C@H]2[C@H](O[C@H]3[C@H](Oc4cc5c(O[C@@H]6O[C@H](CO)[C@@H](O)[C@H](O)[C@H]6O)cc(O)cc5[o+]c4-c4ccc(O)c(O)c4)O[C@H](CO)[C@@H](O)[C@@H]3O)O[C@H](CO)[C@@H](O)[C@@H]2O)cc(OC)c1O]

and (b) a metal ion, wherein Compound I is prepared according to any one of the methods disclosed herein, e.g., by subjecting red cabbage juice or extract to an enzymatic treatment by one or more enzymes, and wherein the edible colorant composition is a solution having a pH of from about 6.0 to about 8.0.

In some embodiments of the edible colorant composition, the one or more enzymes includes one or more esterases. In some embodiments, the one or more esterase is a naturally occurring esterase, e.g., wherein the naturally occurring esterase includes the amino acid sequence of any one of SEQ ID NO: 1-15. In further embodiments, the one or more esterase is a modified esterase. In some embodiments, the modified esterase is a modified carboxylesterase from P. fluorescens. In some embodiments, the modified carboxylesterase from P. fluorescens includes one or more amino acid substitutions at position number 25, 28, 29, 30, 31, 38, 39, 63, 65, 70, 73, 200, or 201 relative to a naturally occurring carboxylesterase from P. fluorescens. In other embodiments, the modified esterase is a modified carboxylesterase from C. salexigens. In some embodiments, the modified carboxylesterase from C. salexigens includes one or more amino acid substitutions at amino acid position number 23, 28, 75, 115, 117, 119, 121, 122, 125, 129, 168, 171, 173, 202, 209, or 212 relative to a naturally occurring carboxylesterase from C. salexigens. In certain embodiments, the modified esterase includes the amino acid sequence of any one of SEQ ID NO: 16-43.

In further embodiments of the edible colorant compositions disclosed herein, Compound I is isolated from the red cabbage juice or extract following the enzymatic treatment. In some embodiments, Compound I is isolated by a purification process including one or more of enzyme precipitation, solid-phase extraction, and preparatory HPLC. In particular embodiments, Compound I is isolated by a purification process including sequentially performing the steps of: (i) enzyme precipitation; (ii) solid-phase extraction; and, optionally, (iii) preparatory HPLC, wherein the preparatory HPLC of step (iii) is performed if the solution includes two or more anthocyanins.

In some embodiments, the edible colorant composition is dried to produce a dry colorant composition. In some embodiments, the edible colorant composition is blue. In particular embodiments, the edible colorant composition has a ΔE value of less than about 17 when compared to an aqueous solution of about 50 ppm to about 100 ppm FD&C Blue No. 1, e.g., wherein the ΔE value is from about 8 to about 10.

In further embodiments, the edible colorant composition is green. In some embodiments, the edible colorant composition further includes a non-artificial yellow colorant selected from the group consisting of safflower, turmeric, beta carotene, and gardenia yellow.

In some embodiments of any of the edible colorant compositions of the present disclosure, the edible colorant composition includes from about 0.5% to about 30% (weight/weight (w/w)) of Compound I. In some embodiments, the edible colorant composition includes from about 0.5% to about 10% (w/w) of Compound I. In some embodiments, the edible colorant composition includes from about 10% to about 30% (w/w) of Compound I. In some embodiments, Compound I is present in an amount greater than about 10% of an anthocyanin chromophore content in the edible colorant composition.

In further embodiments of any of the edible colorant compositions of the present disclosure, the edible colorant composition includes from about 0.3 to about 1.0 molar equivalents of the metal ion to the monoacylated anthocyanin compound. In some embodiments, the metal ion is selected from the group consisting of aluminum (Al³⁺), ferric (Fe³⁺), or ferrous (Fe²⁺) ions.

In another aspect, the present disclosure features an edible product comprising an edible colorant composition of the present disclosure.

In some embodiments, the edible product is a confectionery product. In further embodiments, the edible colorant composition is present in a coating applied to a surface of the confectionery product. In other embodiments, the confectionery product is a confectionery center with a soft-panned or hard-panned sugar-based coating. In some embodiments, the confectionery product is a confectionery center with a soft-panned or hard-panned polyol coating.

In some embodiments, the edible product includes from about 0.0001 to about 10% (w/w) of Compound I. In further embodiments, the edible product includes from about 0.0005 to about 1% (w/w) of Compound I. In further embodiments, the edible product includes from about 0.001 to about 0.5% (w/w) of Compound I.

In another aspect, the present disclosure features an enzyme including the amino acid sequence of any one of SEQ ID NO: 16-43.

In another aspect, the present disclosure features an enzyme including an amino acid sequence including at least one amino acid substitution relative to the amino acid sequence of any one of SEQ ID NO: 1-15. In some embodiments, the enzyme includes an active site motif having at least about 85% (e.g., about 86%, about 87%, about 88%, about 89%, about 90%, about 91%, about 92%, about 93%, about 94%, about 95%, about 96%, about 97%, about 98%, about 99%, or 100%) sequence identity relative to an active site motif of any one of SEQ ID NO: 1-15. In some embodiments, the enzyme includes at least one amino acid substitution relative to the amino acid sequence of SEQ ID NO: 1. For example, the enzyme includes at least one amino acid substitution at amino acid position number 23, 28, 75, 115, 117, 119, 121, 122, 125, 129, 168, 171, 173, 202, 209, or 212 of SEQ ID NO: 1. In other embodiments, the enzyme includes at least one amino acid substitution relative to the amino acid sequence of SEQ ID NO: 2. For example, the enzyme includes at least one amino acid substitution at amino acid position number 25, 28, 29, 30, 31, 38, 39, 63, 65, 70, 73, 200, or 201 of SEQ ID NO: 2. In certain embodiments, the enzyme includes an amino acid substitution at amino acid position number 73 of SEQ ID NO: 2. In particular embodiments, the amino acid substitution at amino acid position number 73 of SEQ ID NO: 2 is a methionine to histidine amino acid substitution.

The foregoing has outlined broadly the features and technical advantages of the present application in order that the detailed description that follows may be better understood. Additional features and advantages of the application will be described hereinafter which form the subject of the claims of the application. It should be appreciated by those skilled in the art that the conception and specific embodiments disclosed may be readily utilized as a basis for modifying or designing other structures for carrying out the same purposes of the present application. It should also be realized by those skilled in the art that such equivalent constructions do not depart from the spirit and scope of the application as set forth in the appended claims. The novel features which are believed to be characteristic of the application, both as to the organization and method of operation, together with further objects and advantages, will be better understood from the following description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A provides high performance liquid chromatography (HPLC) data for red cabbage juice. Each peak corresponds to one of Compounds I, II, A, B, C, D, E, or F, as labeled in the figure.

FIG. 1B provides liquid chromatography-mass spectrometry (LC-MS) data for red cabbage juice. Each peak corresponds to one of Compounds I, II, A, B, C, D, E, or F, as labeled in the figure. The area under each curve is provided below in Table 2.

FIG. 2 provides a comparison of LC-MS data for red cabbage juice before and after enzymatic treatment.

FIG. 3A provides a comparison of the colors provided by whole red cabbage extracts, red cabbage diacylated fraction and Compound I plus metal ion in aqueous solution at pH values ranging from 1 to 9, which demonstrates the lower violet color contribution, e.g., at pH 7.

FIG. 3B provides a visual comparison of the color provided by an FD&C Blue No. 1 (Blue 1) extract in aqueous solution.

FIG. 3C provides a visual comparison of the colors provided by Spirulina Blue extracts in aqueous solution at concentrations from 0.005% to 0.04%.

FIG. 4A provides a high performance liquid chromatography (HPLC) trace of red cabbage extract in buffer (red), incubated with 0.25 mg/mL 1AUR-WT (blue), and incubated with 0.25 mg/mL 1AUR-M73H (black) after 24 hours.

FIG. 4B provides a graph showing the completion of conversion to Compounds I and II over a 24 hour reaction with enzymes 1AUR-WT and 1AUR-M73H. Each data point of the 24 hour HPLC trace of FIG. 4A corresponds with a data point in FIG. 4B.

### DETAILED DESCRIPTION OF THE INVENTION

The presently disclosed subject matter relates to, inter alia, an enzymatic treatment that converts one or more anthocyanin naturally occurring in a fruit or vegetable juice or extract into an anthocyanin molecule having desirable colorant properties, i.e., Compound I. The enzymes disclosed herein catalyze the cleavage of one or more chemical bonds in an anthocyanin, e.g., one or more acyl groups in an anthocyanin. Enzymatic treatment of a solution containing a mixture of anthocyanins, such as a fruit or vegetable juice or extract, results in the increase in the amount of one or more specific anthocyanins in the mixture, i.e., Compound I and/or Compound II. The desired compounds, i.e., Compound I, can then be separated from the other compounds, i.e., Compound II, in the solution following enzymatic treatment.

Further disclosed herein are methods for the preparation of the enzymes used in the disclosed enzymatic treatment, as well as methods for the separation and isolation of anthocyanins from a solution including a mixture of anthocyanins. Additionally, the disclosed subject matter provides for the use of an anthocyanin as a colorant in edible food products.

### 1. Definitions

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art.

As used herein, the term “anthocyanin” refers to a water soluble pigment that is generally red, purple or blue, depending on the pH of a solution including the anthocyanin. Anthocyanins are the glucosides of anthocyanidins. Anthocyanidins are generally flavylium cation derivatives of anthocyanins (devoid of the sugar moieties) and can include aurantinidin, cyanidin, dephininidin, europinidin, luteolinidin, pelargonidin, malvidin, peonidin, petunidin and rosinidin, for example. Cyanidin is the exemplary anthocyanidin.

As used herein, the term “acylated anthocyanin” refers to an anthocyanin molecule having one or more acyl molecules esterified to one or more sugar molecules that are attached to the anthocyanidin. Acyl molecules or groups can include cinnamate derivative and or hydroxyl benzoyl derivatives. Cinnamate derivatives can include ferulic, para coumaric or sinapic groups. Sinapic is the exemplary acyl group.

As used herein, the term “monoacylated anthocyanin” refers to an anthocyanin molecule that includes one acyl group linked by an ester bond to a sugar group of the anthocyanin molecule.

As used herein, the term “diacylated anthocyanin” refers to an anthocyanin molecule that includes two acyl groups linked by ester bonds to one or more sugar groups of the anthocyanin molecule.

As used herein, “sugar syrup” refers to a liquid material comprising at least a sugar and water. In certain embodiments, a sugar syrup can include a syrup where a sugar is dissolved in the water in an amount of at least about 60% sugar solids by weight of the syrup. In certain embodiments, other components can also be present within the sugar syrup. For example, and not by way of limitation, an edible colorant composition of the present disclosure can be used in a sugar syrup, e.g., to add a color to the sugar syrup.

As used herein, “coating layer” refers to a layer obtained by one application of a coating material, e.g., a sugar syrup, to a substrate, e.g., a food product, being coated and which is dried and crystallized.

As used herein, “coating” refers to the total amount of coating material, e.g., one or more sugar syrups, applied to a substrate, e.g., a food product, which is dried and crystallized after each application, upon completion of a coating process. In certain embodiments, the coating process can include one or more steps of applying a coating material and drying and crystallizing each applied coating layer, e.g., a sugar syrup containing one or more colorant compositions of the present disclosure, to the substrate.

As used herein, the term “colorant” refers to any substance that imparts color by absorbing or scattering light at different wavelengths.

As used herein, the term “colorant composition” refers to any composition that imparts color by absorbing or scattering light at different wavelengths.

As used herein, the term “non-artificial colorant” refers to any substance that exists in or is produced by nature, or is obtained from a non-artificial source. In certain embodiments, the term “non-artificial colorant” refers to a colorant that comprises one of more anthocyanins obtained from a non-artificial source, e.g., a vegetable, a plant, or a flower (or a flower petal).

As used herein, the term “non-artificial colorant composition” refers to any composition that comprises a colorant that exists in or is produced by nature or is obtained from a non-artificial source. In certain embodiments, the term “non-artificial colorant composition” refers to a colorant composition that comprises one of more anthocyanins obtained from a non-artificial source, e.g., a vegetable, a plant, or a flower (or a flower petal).

As used herein, “confectionery product” or “confection” refers to a sweet or candy food product. Non-limiting examples of confectionery products include cakes, cookies, pies, candies, chocolates, chewing gums, gelatins, ice creams, puddings, jams, jellies, and other condiments, cereal, and other breakfast foods, canned fruits and fruit sauces. As used herein, the confectionery products having neutral pH (e.g., about pH 5-8 or from about pH 6 to about pH 8) are particularly suitable for the colorant compositions disclosed herein.

As used herein, “maximum absorbance,” “lambda max,” or “λₘₐₓ,” refers to the wavelength in nanometers at which the maximum fraction of light is absorbed by a substance, colorant and/or colorant composition.

As used herein, “FD&C Blue No. 1” includes the various names given to the identical artificial blue colorant, Brilliant Blue FCF and European Commission E133. The lambda max of FD&C Blue No. 1 is 630 nm. FD&C Blue No. 1 is used interchangeably with Cyan Blue or FD&C Blue No. 1.

As used interchangeably herein, the terms “color” and “color characteristics” refer to the color properties such as hue, chroma, purity, saturation, intensity, vividness, value, lightness, brightness and darkness, and color model system parameters used to describe these properties, such as Commission Internationale de l'Eclairage CIE 1976 CIELAB color space L*a*b* values and CIELCH color space L*C*h° values. The CIELAB and CIELCH color models provide more perceptually uniform color spaces than earlier color models. In certain embodiments, the colorants of the present disclosure can be analyzed with a spectrophotometer, and CIELAB L*a*b* and CIELCH L*C*h° values can be calculated from the spectral data, as described in greater detail below. The L*a*b* and L*C*h° values provide a means of representing color characteristics and assessing the magnitude of difference between two colors. Methods for determining the CIELAB and CIELCH values of colorants are disclosed in International Patent Publication Nos. WO 2014/150230 and WO 2014/152417, the contents of which are hereby incorporated by reference in their entireties. CIELAB color space L*a*b values and CIELCH color space L*C*h values can be expressed using three-dimensional representations and also two-dimensional representations where the 3^{rd} dimension is fixed. An example of the latter is a two-dimensional cross-sectional representation of the L*a*b* space at a specific L* value, for example at L*=50. Such a representation allows a useful display of colorants in the a*b* space with the caveat that some points are actually above, on or below the plane shown. The L* value is typically chosen to be at a suitable midpoint between the data points being shown.

The L*a*b* and L*C*h° values also provide a means of representing color characteristics and assessing the magnitude of difference between two colors not only of solutions, but also of products. Measurements of products are accomplished using reflectance measurements from the surface of the product, for example, the surface of a hard panned confection. In the case of reflectance measurements, the L*a*b* and L*C*h° values reported herein were calculated based on spectral data obtained with a Konica Minolta Spectrophotometer CM-3500d operated in reflectance mode with a D65 illuminant and 10° observer angle.

The term “reflectance” as used herein with respect to a material is the percentage of any incident electromagnetic radiation that reflects back from a surface. Reflectance is a function of wavelength, and the reflectance of a material can vary across the electromagnetic spectrum. A material that is a perfect reflector at a particular wavelength has a reflectance of 100% at that wavelength.

As used herein, “hue” or “hue angle” refers to the color property that gives a color its name, for example, red, blue and violet.

As used herein, “chroma” is a color property indicating the purity of a color. In certain embodiments, a higher chroma is associated with greater purity of hue and less dilution by white, gray or black.

As used herein, “value” is a color property indicating the lightness or darkness of a color wherein a higher “value” is associated with greater lightness.

As used herein “admixing,” for example, “admixing a colorant composition of the present disclosure with a food product,” refers to a method where a colorant composition of the present disclosure is mixed with or added to the completed product or mixed with some or all of the components of the product during product formation or some combination of these steps. When used in the context of admixing the term “product” refers to the product or any of its components. Admixing can include a process that includes adding the colorant composition to the product, spraying the colorant composition on the product, coating the colorant composition on the product, painting the colorant composition on the product, pasting the colorant composition on the product, encapsulating the product with the colorant composition, mixing the colorant composition with the product or any combination thereof. The colorant compositions, e.g., those that are admixed with the product, can be a liquid, dry powder, spray, paste, suspension or any combination thereof. In certain embodiments, the term “admixing” can refer to mixing Compound I as disclosed herein with one or more additional components to create a finished colorant.

As used herein, “solution,” refers to a liquid mixture in which the minor component (the solute) is uniformly distributed within the major component (the solvent). For example, a monoacylated anthocyanin compound (Compound I) is distributed within a solution, such as a sugar syrup, to yield a blue colored coating on a confectionery substrate.

As used herein, the phrase “consumer product” or “end product” refers to composition that is in a form ready for use by the consumer for the marketed indication. A solvent suitable for use in a consumer product is a solvent that, when combined with other components of the end product, will not render the consumer product unfit for its intended consumer use.

As used herein, the term “esterase” refers to an enzyme that catalyzes the hydrolysis of an ester into an acid and an alcohol. A “modified esterase” refers to an esterase that includes modifications, e.g., one or more amino acid substitutions, relative to a naturally occurring esterase.

As used herein, the term “extract” refers to substance made by extracting a part of a raw material, such as a fruit or vegetable, by using a solvent such as but not limited to ethanol or water.

As used herein, “food grade,” refers to any substance, metal ion and/or colorant composition that is of a grade acceptable for use in edible food products.

As used herein, “food product” refers to an ingestible product, such as, but not limited to, human food, animal foods and pharmaceutical compositions.

### 2. Anthocyanins and Their Color Properties

The present disclosure relates to the use of an anthocyanin colorant, i.e., Compound I, for use in an edible colorant composition, as well as edible food products containing the edible colorant composition. Compound I can be formed by enzymatic treatment with an enzyme as described herein of a solution including an anthocyanin or a mixture of anthocyanins, such as those naturally occurring in a fruit or vegetable juice or extract. The disclosed enzymes catalyze the hydrolysis of an ester bond to remove an acyl group from an anthocyanin, thereby resulting in the production of Compound I. Enzymatic treatment of a solution including a mixture of anthocyanins also results in the reduction of the total number of anthocyanins present in the solution, e.g., a fruit or vegetable juice or extract, thereby allowing for simplified purification of Compound I from the solution.

### 2.1. Anthocyanins

The anthocyanins of the present disclosure can be obtained from natural sources and/or juices or extracts thereof. For example, and not by way of limitation, the anthocyanins of the present disclosure can be obtained from vegetables (or extracts or juices thereof) such as red cabbage, purple sweet potato, red potato, blue potato, red radish, black carrot, purple carrot, purple corn, red corn, red onion, purple broccoli, red broccoli, purple cauliflower, rhubarb, black bean, red leaf lettuce, black rice, eggplant or combinations thereof. In certain embodiments, the anthocyanins can be obtained from fruits (including extracts or juices thereof) such as, but not limited to, strawberry, raspberry, cranberry, lingonberry, red grape, apple, black currant, red currant, cherry, blueberry, elderberry, bilberry, crowberry, blackberry, chokeberry, gooseberry, acai, nectarine, peach, plum, blood orange, blue tomato or combinations thereof. In certain embodiments, the anthocyanins of the present disclosure can be obtained from flower petals (including extracts or juices thereof) such as those of “Heavenly Blue” Morning Glory, “Better Times” Rose or combinations thereof.

In certain embodiments, a vegetable, fruit, and flower petal juice can be obtained by pressing liquid out of the fruit, vegetable, or flower. In certain embodiments, a vegetable, fruit, and flower petal extract can be obtained by washing a macerated fruit, vegetable, or flower with a solvent (e.g., water or alcohol). Juices and extract can contain anthocyanins as well as other naturally occurring compounds, including, for example, carbohydrates, acids, flavonoids, metal ions, phenolic acids, phenolic acid esters, and vitamins.

In certain embodiments, vegetable, fruit, and flower petal juices or extracts can include processed juices and extracts. Non-limiting examples of processed juices and extracts include reconstituted juices and extracts, deodorized juices and extracts, and juices and extracts subjected to other processes for removing specific or broad classes of compounds. In certain embodiments, the present disclosure is directed to enzymatic treatment of anthocyanins derived from red cabbage extracts, e.g., from red cabbage juice.

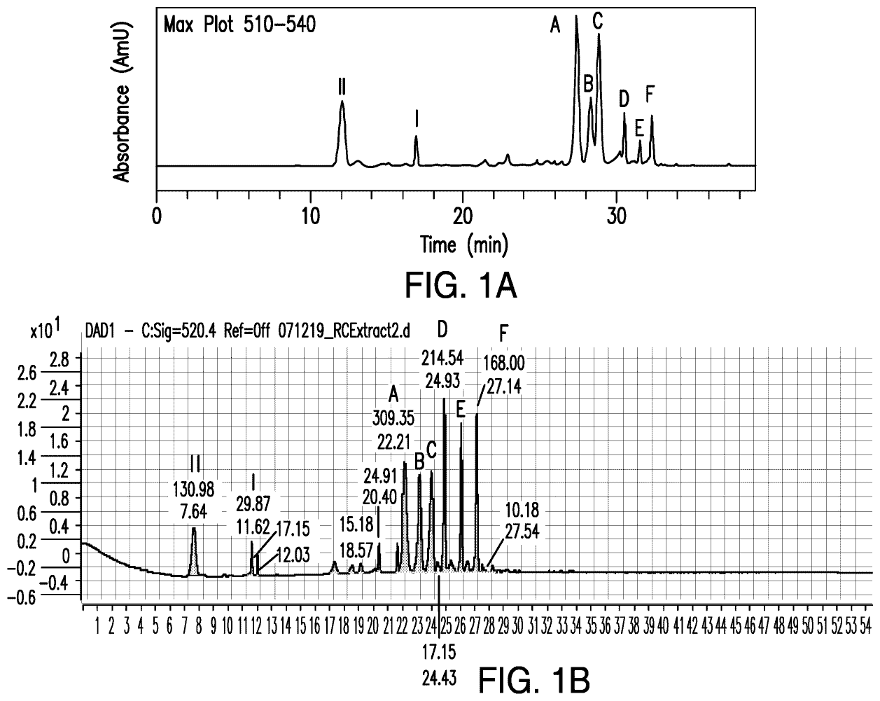

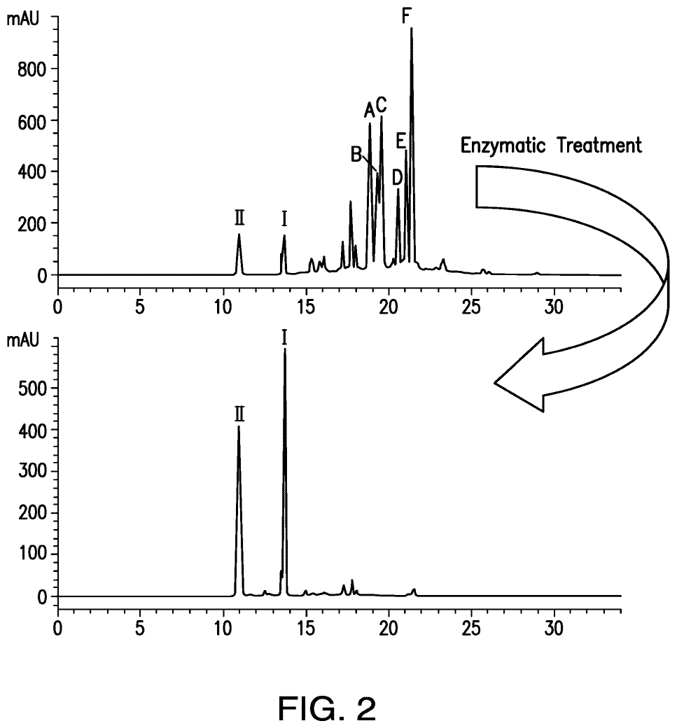

Red cabbage juice contains a wide variety of compounds and anthocyanins as can be seen in the HPLC and LC-MS data shown in FIGS. 1A and 1B. Eight anthocyanins, referred to herein as Compounds I, II, and A-F, have been identified as naturally occurring in red cabbage juice or extract. The structures of these anthocyanins can be generally described by Formula 1:

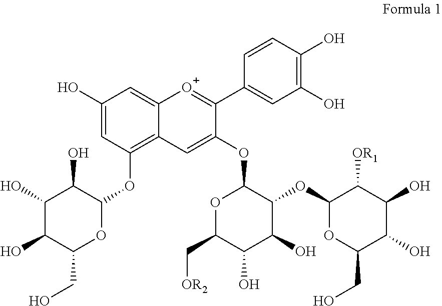

wherein each of R₁ and R₂ is, independently, H or an acyl group. An acyl group is generally represented by the formula R—C═O. For example, when R₁ and/or R₂ is an acyl group, the acyl group can have the structure of

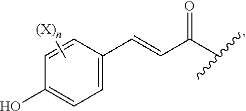

wherein X is a C₁₋₄ alkoxy group and n is an integer from 0-2. In some embodiments, X is a methoxy group. In some embodiments, n is 0, 1, or 2. In some embodiments, each of R₁ and R₂ is, independently, a para coumaric group, a ferulic group, or a sinapic group.

Compound II is a non-acylated anthocyanin, wherein R₁ and R₂ are both H; Compound I and Compounds A-C are monoacylated anthocyanins, i.e., wherein R₁ is H and R₂ is an acyl group selected from a para coumaric group, a ferulic group, or a sinapic group; and Compounds D-F are diacylated anthocyanins, i.e., wherein each of R₁ and R₂ is an acyl group selected from a para coumaric group, a ferulic group, or a sinapic group. The chemical structures of these compounds are provided below:

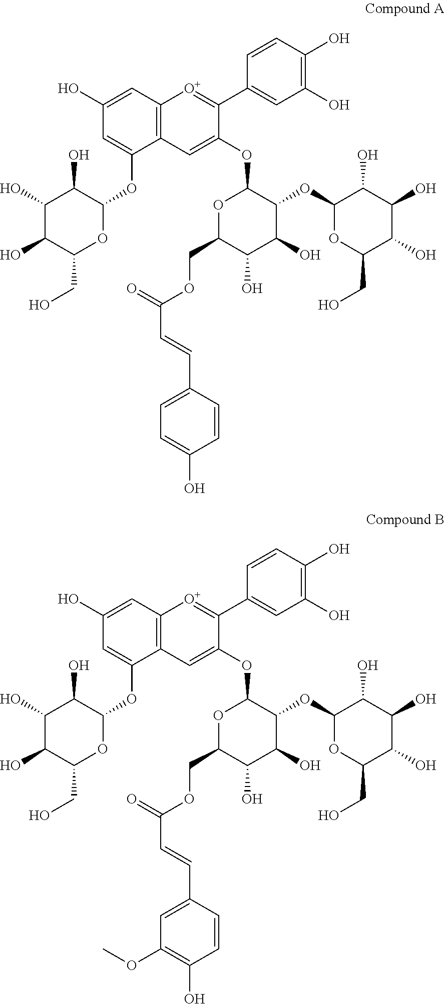 [SMILES: O=C(/C=C/c1ccc(O)cc1)OC[C@H]1O[C@@H](Oc2cc3c(O[C@@H]4O[C@H](CO)[C@@H](O)[C@H](O)[C@H]4O)cc(O)cc3[o+]c2-c2ccc(O)c(O)c2)[C@H](O[C@@H]2O[C@H](CO)[C@@H](O)[C@H](O)[C@H]2O)[C@@H](O)[C@@H]1O ; COc1cc(/C=C/C(=O)OC[C@H]2O[C@@H](Oc3cc4c(O[C@@H]5O[C@H](CO)[C@@H](O)[C@H](O)[C@H]5O)cc(O)cc4[o+]c3-c3ccc(O)c(O)c3)[C@H](O[C@@H]3O[C@H](CO)[C@@H](O)[C@H](O)[C@H]3O)[C@@H](O)[C@@H]2O)ccc1O]

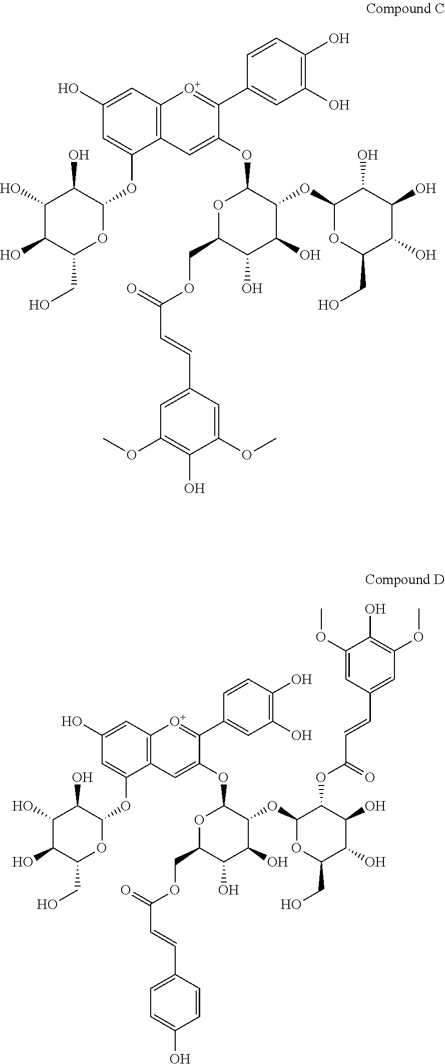 [SMILES: COc1cc(/C=C/C(=O)OC[C@H]2O[C@@H](Oc3cc4c(O[C@@H]5O[C@H](CO)[C@@H](O)[C@H](O)[C@H]5O)cc(O)cc4[o+]c3-c3ccc(O)c(O)c3)[C@H](O[C@@H]3O[C@H](CO)[C@@H](O)[C@H](O)[C@H]3O)[C@@H](O)[C@@H]2O)cc(OC)c1O ; COc1cc(/C=C/C(=O)O[C@H]2[C@H](O[C@H]3[C@H](Oc4cc5c(O[C@@H]6O[C@H](CO)[C@@H](O)[C@H](O)[C@H]6O)cc(O)cc5[o+]c4-c4ccc(O)c(O)c4)O[C@H](COC(=O)/C=C/c4ccc(O)cc4)[C@@H](O)[C@@H]3O)O[C@H](CO)[C@@H](O)[C@@H]2O)cc(OC)c1O]

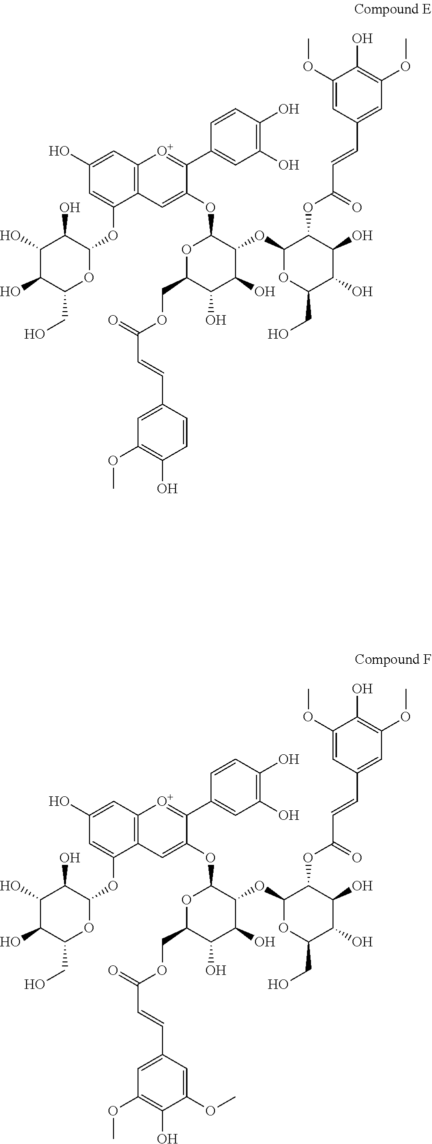 [SMILES: COc1cc(/C=C/C(=O)OC[C@H]2O[C@@H](Oc3cc4c(O[C@@H]5O[C@H](CO)[C@@H](O)[C@H](O)[C@H]5O)cc(O)cc4[o+]c3-c3ccc(O)c(O)c3)[C@H](O[C@@H]3O[C@H](CO)[C@@H](O)[C@H](O)[C@H]3OC(=O)/C=C/c3cc(OC)c(O)c(OC)c3)[C@@H](O)[C@@H]2O)ccc1O ; COc1cc(/C=C/C(=O)OC[C@H]2O[C@@H](Oc3cc4c(O[C@@H]5O[C@H](CO)[C@@H](O)[C@H](O)[C@H]5O)cc(O)cc4[o+]c3-c3ccc(O)c(O)c3)[C@H](O[C@@H]3O[C@H](CO)[C@@H](O)[C@H](O)[C@H]3OC(=O)/C=C/c3cc(OC)c(O)c(OC)c3)[C@@H](O)[C@@H]2O)cc(OC)c1O]

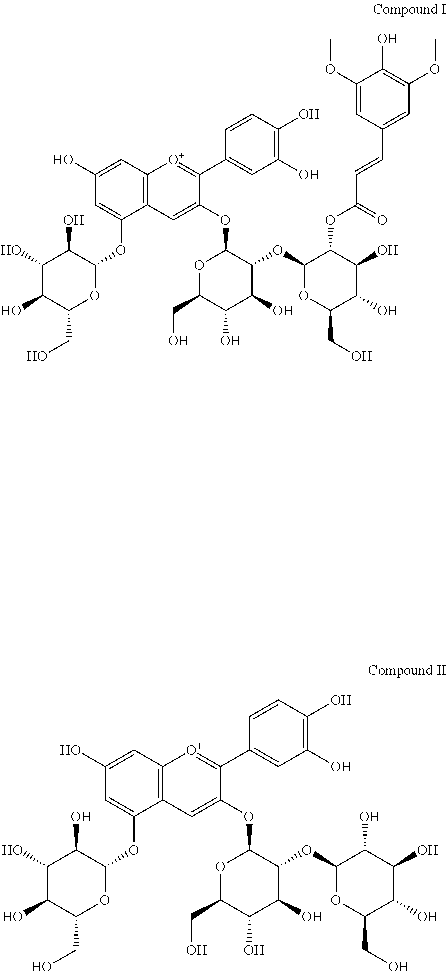 [SMILES: COc1cc(/C=C/C(=O)O[C@H]2[C@H](O[C@H]3[C@H](Oc4cc5c(O[C@@H]6O[C@H](CO)[C@@H](O)[C@H](O)[C@H]6O)cc(O)cc5[o+]c4-c4ccc(O)c(O)c4)O[C@H](CO)[C@@H](O)[C@@H]3O)O[C@H](CO)[C@@H](O)[C@@H]2O)cc(OC)c1O ; OC[C@H]1O[C@@H](Oc2cc(O)cc3[o+]c(-c4ccc(O)c(O)c4)c(O[C@@H]4O[C@H](CO)[C@@H](O)[C@H](O)[C@H]4O[C@@H]4O[C@H](CO)[C@@H](O)[C@H](O)[C@H]4O)cc23)[C@H](O)[C@@H](O)[C@@H]1O]

Compounds A-C have the same base structure as Compound II but include a single additional acyl group, such as a ferulic, a para coumaric, or a sinapic group, at the R₂ position. Similarly, Compounds D-F have the same base structure as Compound I, but further include an additional acyl group, such as a ferulic, a para coumaric, or a sinapic group, at the R₂ position. The LC-MS data and identity of Compounds I, II, and A-F are provided in Table 1.

TABLE 1
Anthocyanins present in Red Cabbage Juice

| Com- | RT | λvis | λacyl | | | |
|---|---|---|---|---|---|---|
| pound | (mm) | (nm) | (nm) | M⁺ | R₁ | R₂ |
| II | 12.1 | 513 | — | 773 (287) | H | H |
| I | 17.0 | 528 | 334 | 979 (287) | Sinapic | H |
| A | 27.4 | 523 | 313 | 919 (287) | H | p-Coumeric |
| B | 28.3 | 523 | 326 | 949 (287) | H | Ferulic |
| C | 28.9 | 524 | 329 | 979 (287) | H | Sinapic |
| D | 30.5 | 536 | 319 | 1125 (287) | Sinapic | p-Coumeric |
| E | 31.6 | 536 | 330 | 1155 (287) | Sinapic | Ferulic |
| F | 32.3 | 536 | 331 | 1185 (287) | Sinapic | Sinapic |

In certain embodiments, the present disclosure is directed to preparation and isolation of Compound I. The chemical name of Compound I is 3-O-(2-O-(2-O-(E)-sinapoyl-β-D-glucopyranosyl)-(β-D-glucopyranosyl)-5-O-β-D-glucopyranosylcyanidin. Compound I is a monoacylated anthocyanin that demonstrates a unique ability to generate a cyan blue hue under certain conditions. It is a minor component of red cabbage juice or extract. Red cabbage juice or extract naturally only comprises from about 1% to about 4% of Compound I. The HPLC spectrum of FIG. 1A and Table 2 below show the percentage amounts of each of Compounds I, II, and A-F in one exemplary extract of red cabbage juice. In this example, Compound I is only present in an amount of about 2%.

TABLE 2
Composition of Red Cabbage Juice

| | | % of |
|---|---|---|
| Peak | | Total |
| (Compound) | Area | Area |
| II | 130.98 | 8.85 |
| I | 29.87 | 2.02 |
| A | 309.35 | 20.91 |
| B | 237.86 | 16.07 |
| C | 233.81 | 15.80 |
| D | 214.54 | 14.50 |
| E | 155.29 | 10.49 |
| F | 168.00 | 11.35 |
| Total | 1479.70 | |

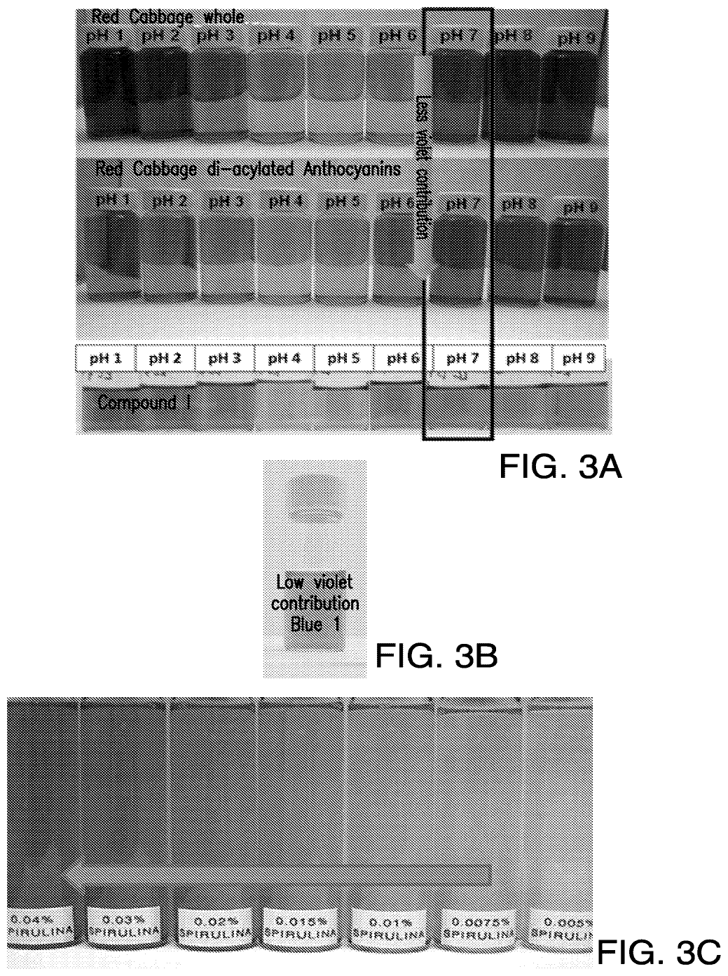

Color characteristics of Compound I can change in the presence of a metal ion in a solution thereof. For instance, a colorant comprising Compound I and about 1.0 molar equivalents or about 0.3 molar equivalents of a metal ion (such as Al³⁺ or Fe³⁺) at about pH 5 to 8 surprisingly has less of a violet hue (i.e., lower violet contribution) than red cabbage juice at about pH 7 or 8, the diacylated anthocyanin fraction of red cabbage juice at about pH 7 or 8, and Spirulina Blue. A comparison of the color at about pH 7 for red cabbage, di-acylated red cabbage anthocyanin fraction (Compounds D-F) and Compound I with a metal ion is provided in FIG. 3A. FIG. 3A illustrates that violet contribution in the Compound I solution with metal ion is significantly reduced. By comparison, the violet contribution is higher for the Spirulina Blue solution, and the violet contribution even increases as the concentration of Spirulina Blue is increased (FIG. 3C). The solution of Compound I with metal ion is the closest to a solution of FD&C Blue No. 1 (FIG. 3B) in having a low violet contribution. Solutions of Compound I with metal ion are particularly unique in demonstrating hue angle, wavelength, and a* and b* values comparable to those of solutions of FD&C Blue No. 1.

A colorant comprising Compound I and a metal ion at about pH 5 to 8 provides significantly less violet hue in its blue color, which is critical when blending with a non-artificial yellow color to produce a bright and true green color. Other non-artificial blue colorants having a significant violet hue tend to produce undesirable green colors when mixed with non-artificial yellow colorants. These undesirable green colors are typically characterized as being a muddy green or olive-green color.

### 2.2 Colorant Compositions

An anthocyanin as disclosed herein, i.e., Compound I, can be used in a colorant composition. In certain embodiments, a colorant composition of the present disclosure can be a cyan blue colorant composition that includes, e.g., Compound I with a metal ion at a pH of about 6 to about 8.

In certain embodiments, one or more of the colorant compositions of the present disclosure can be added to a food product, in an amount effective to increase, enhance and/or modify the color characteristics of a food product or portion thereof. For example, and not by way of limitation, a colorant composition of the present disclosure can enhance the blue color characteristics of the food product (or portion thereof). In certain embodiments, colorant compositions of the present disclosure can be used to increase, enhance and/or modify the color characteristics of a food product (or portion thereof), such as, but not limited to, a chocolate confection. In certain embodiments, the colorant composition is a liquid. In other embodiments, the colorant composition is a solid created, for example, by drying a liquid colorant composition.

In certain embodiments, the colorant composition of the present disclosure comprises Compound I. In certain embodiments, the colorant composition comprises from about 0.005% to about 100%, or from about 0.005% to about 80%, or from about 0.005% to about 60%, or from about 0.005% to about 50%, or from about 0.005% to about 40%, or from about 0.005% to about 30%, or from about 0.005% to about 20%, or from about 0.005% to about 10%, or from about 0.5% to about 30%, or from about 0.5% to about 5%, or from about 0.5% to about 15%, or from about 10% to about 25%, or from about 20% to about 30% by weight of Compound I. In certain embodiments, the colorant composition comprises from about 1% to about 80%, or from about 10% to about 70% by weight of the colorant composition, or from about 20% to about 60% by weight of Compound I. In certain embodiments, the colorant composition comprises from about 30% to about 50% by weight of Compound I. In certain embodiments, the colorant composition comprises from about 40% to about 50% by weight of Compound I. In certain embodiments, the colorant composition comprises from about 0.005% to about 10%, from about 0.005% to about 1%, or from about 0.005% to about 0.1% by weight of Compound I. In certain embodiments, the colorant composition comprises greater than about 0.5%, greater than about 1%, greater than about 5%, greater than about 10%, greater than about 15%, greater than about 20%, greater than about 25%, or greater than about 30% by weight of Compound I.

In certain embodiments, Compound I makes up greater than about 10% of the anthocyanin chromophore portion of a colorant, preferably greater than about 20%, preferably greater than about 30%, preferably greater than about 40%, preferably greater than about 50%, preferably greater than about 60%, preferably greater than about 70%, preferably greater than about 80%, preferably greater than about 90%, preferably greater than about 95%, and most preferably about 100% of the anthocyanin chromophore portion of a colorant. In further embodiments, Compound I is present in an amount of from about 0.05% to about 0.1% (w/w) (e.g., from about 0.05% to about 0.09% (w/w), from about 0.06% to about 0.08% (w/w), or from about 0.07% to about 0.1% (w/w)) of an anthocyanin chromophore content of the colorant composition. In other embodiments, Compound I is present in an amount of from about 5% to about 35% (w/w) (e.g., from about 5% to about 15% (w/w), from about 5% to about 20% (w/w), from about 5% to about 30% (w/w), from about 10% to about 25% (w/w), from about 10% to about 35% (w/w), from about 20% to about 35% (w/w)) of an anthocyanin chromophore content of the colorant composition.

### 2.3 pH

In certain embodiments, the pH of the colorant composition of the present disclosure comprises at least one pH adjusting ingredient. The pH of the colorant composition can be adjusted using food grade acids or bases (e.g., food grade sodium hydroxide) and/or the pH can be adjusted with the use of food grade buffers (e.g., phosphate buffer) or food grade acids or bases can be used in combination with food grade buffers to adjust pH.

Non-limiting examples of pH adjusting ingredients include potassium and sodium-based buffers. In certain embodiments, the pH adjusting ingredient can include potassium phosphate, sodium acetate, or other food grade buffers known in the art. Another pH adjustment component can be, for example, sodium hydroxide, which is a source of an alkali metal ion.

In certain embodiments, the colorant composition has a pH of about 5.0 to about 8.0. In certain embodiments, for a blue colorant composition, the pH can be from about 5.0 to about 8.0, or from about 6.0 to about 8.0, or from about 7.0 to about 8.0. In certain non-limiting embodiments, the pH of the pH adjusting ingredient present within a colorant composition, e.g., a blue colorant composition, is about 6.0. In certain embodiments, the pH of the pH adjusting ingredient present within a colorant composition, e.g., a blue colorant composition, is about 6.5. In certain embodiments, the pH of the pH adjusting ingredient present within a colorant composition, e.g., a blue colorant composition, is about 7.0. In certain embodiments, the pH of the pH adjusting ingredient present within a colorant composition, e.g., a blue colorant composition, is about 7.5. In certain embodiments, the pH of the pH adjusting ingredient present within a colorant composition, e.g., a blue colorant composition, is about 8.0.

### 2.4 Metal Ions

In certain embodiments of the present disclosure, the colorant composition further comprises at least one metal ion or cation or salt form thereof. In certain embodiments, the metal ion can bind to one or more hydroxyl groups present on the B-ring of the cyanidin chromophore portion of the anthocyanin molecule. Complexation of Compound I with a metal ion, such as Al³⁺, causes a bathochromic shift in the color to produce the true cyan blue color of the color composition. Other exemplary metal ions that can cause a bathochromic shift to produce a true cyan blue color include Fe²⁺ and Fe³⁺, as well as other metal ions disclosed herein.

In certain embodiments, the metal ion is a multivalent metal ion such as, but not limited to, a divalent metal ion or a trivalent metal ion. A non-limiting example of a divalent cation include Fe²⁺. In certain embodiments, the metal cation is a trivalent cation such as Al³⁺ or Fe³⁺. In certain embodiments, the metal ion is Al³⁺. In certain embodiments, the metal ion is Fe²⁺. In certain embodiments, the metal ion is Fe³⁺.

In certain embodiments, the metal ion is present in the colorant composition as a metal salt. For example, and not by way of limitation, the metal salt is AlCl₃, Al₂(SO₄)₃, FeCl₃, or FeCl₂.

In certain embodiments, there can be a food grade metal ion or salt form thereof. In certain embodiments, the metal ion or salt form thereof can be selected to be suitable for use in an edible product, e.g., Al³⁺ or Fe³⁺.

In certain embodiments, the molar ratio of Compound I to metal ion is about 1:100, or about 1:10, or about 1:1, or about 1:0.5, or about 1:0.3, or about 1:0.25, or about 1:0.1, or about 1:0.01, or about 1:0.001, or about 1:0.1.

### 2.5 Combinations of Colorant Compositions

The present disclosure also provides for colorant compositions that include a combination of colorants. In certain non-limiting examples, the blue colorant as disclosed herein can be combined with one or more different yellow colorants to obtain a bright and true green colorant. A desirable bright and true green color is obtained by the combination of FD&C Yellow No. 5 and FD&C Blue No. 1. A mixture of FD&C Yellow No. 5 and FD&C Blue No. 1 at a 4:1 ratio (by weight) in solution, when applied in food products, gives a particularly desirable green color. Such an artificial green color applied to a hard panned confection showed an L*=60, a*=−46, b*=43, C*=63, and h°=137, and produces a desirable and true green color in a confectionery product. Heretofore, it has been difficult to achieve a color close or similar to the color achieved with the artificial colors noted.

The color of non-artificial colored products, for example hard panned candies, can be measured and compared to values of an ideal colored product from artificial colors. It is also possible to calculate the ΔE values for non-artificial colored candies relative to the artificial reference. For example, a product colored with red cabbage at pH 8 plus turmeric has a ΔE of 52 compared to the artificial reference, while the Spirulina plus turmeric panned candy has a ΔE of 31 compared to the artificial reference candy. A panned candy colored with Spirulina plus turmeric currently represents the state of the art for producing a green color using non-artificial colorants; these data show that non-artificial products are still notably different from their artificial counterparts.

In certain embodiments, Compound I at about pH 7 plus from about 0.3 to about 1.0 mol. equiv. (e.g., about ⅓ mol. equiv. or about 1 mol. equiv.) of Al³⁺, Fe²⁺, or Fe³⁺ provides color characteristics as discussed below that mimic closely those characteristics of FD&C Blue No. 1 (cyan blue).

Such color characteristics combined with yellow colorants can lead to a bright and true green hue. Exemplary non-artificial yellow colorants useful for the edible colorant compositions described herein include, but are not limited to, curcuminoids (e.g., from turmeric), carotenoids (e.g., from saffron, gac fruit, and gardenia), annatto (e.g., from achiote) and combinations thereof. For instance, the non-artificial yellow colorant can be turmeric, safflower (Carthamus), beta carotene, or gardenia yellow. In certain embodiments, safflower is used as the non-artificial yellow colorant to produce a bright and true green hue.

Preferably, the non-artificial yellow colorant has a hue angle of about 90° or greater. At hue angles greater than about 90°, the yellow colorant includes comparatively less red and more green in the undertones, which results in a better green colorant composition when mixed with blue. At hue angles below about 90°, the yellow colorant includes comparatively more red, which results in a muddier green due to brown undertones in the green. Exemplary non-artificial yellow colorants that may be useful for the edible colorant compositions are provided below in Table 3. Values are taken from the non-artificial yellow colorants at different concentrations on finished panned candies.

TABLE 3
Non-artificial yellow colorants on panned candies

| | Hue angle |
|---|---|
| Different colorant | of finished |
| powders and use rates | panned candies |
| Beta-Carotene 1 (0.5%) | 75.51 |
| Beta-Carotene 2 (0.5%) | 84.10 |
| Beta-Carotene 3 (0.01%) | 95.03 |
| Beta-Carotene 3 (0.03%) | 90.84 |
| Beta-Carotene 4 (0.1%) | 87.70 |
| Beta-Carotene 4 (0.05%) | 91.41 |
| Safflower 1 (0.8%) | 90.39 |
| Safflower 2 (1%) | 104.68 |
| Safflower 3 (1%) | 93.71 |
| Safflower 4 (2%) | 100.33 |
| Safflower 5 (2%) | 90.24 |
| Safflower 6 (1%) | 85.66 |
| Safflower 7 (0.5%) | 95.64 |
| Safflower 7 (0.25%) | 101.06 |
| Safflower 8 (1.5%) | 92.72 |
| Lutein 1 (0.5%) | 72.26 |
| Lutein 1 (0.1%) | 87.70 |
| Lutein 1 (0.05%) | 88.81 |
| Curcumin 1 No. 26295 (1%) | 80.58 |
| Curcumin 2 (0.2%) | 90.69 |
| Curcumin 2 (0.4%) | 87.09 |
| Curcumin 3 | 93.73 |
| Curcumin 4 (0.2%) | 83.41 |
| Curcumin 4 (0.25%) | 81.98 |
| FD&C-Yellow 5 (0.2%) synthetic | 81.29 |
| FD&C-Yellow 5 (0.1%) synthetic | 86.45 |
| FD&C-Yellow 5 (0.05%) synthetic | 91.53 |
| FD&C-Yellow 5 (0.0282%) synthetic | 93.19 |
| FD&C-Yellow 5 (0.02%) synthetic | 96.26 |
| Gardenia Yellow (0.4%) | 82.40 |

Further non-artificial yellow colorants known in the art aside from those listed in the above table may also be useful for the edible colorant compositions disclosed herein.

### 2.6 Color Characteristics

As embodied herein, color characteristics of the presently-disclosed non-artificial blue anthocyanin-containing colorants, can be determined. Such color characteristics can include hue, chroma, purity, saturation, intensity, vividness, value, lightness, brightness and darkness, and color model system parameters used to describe these properties, such as Commission Internationale de l'Eclairage CIE 1976 CIELAB color space L*a*b* values and CIELCH color space L*C*h° values.

For example, L*a*b* values consist of a set of coordinate values defined in a three-dimensional Cartesian coordinate system. L* is the value, or lightness, coordinate. L* provides a scale of lightness from black (0 L* units) to white (100 L* units) on a vertical axis, a* and b* are coordinates related to both hue and chroma, a* provides a scale for greenness (−a* units) to redness (+a* units), with neutral at the center point (0 a* units), on a horizontal axis; b* provides a scale for blueness (−b* units) to yellowness (+b* units), with neutral at the center point (0 b* units), on a second horizontal axis perpendicular to the first horizontal axis. The three axes cross where L* has a value of 50 and a* and b* are both zero.

L*C*h° values consist of a set of coordinate values defined in a three-dimensional cylindrical coordinate system. L* is the value, or lightness, coordinate. L* provides a scale of lightness from black (0 L* units) to white (100 L* units) on a longitudinal axis. h° is the hue coordinate. h° is specified as an angle from 0° to 360° moving counterclockwise around the L* axis. Pure red has a hue angle of 0°, pure yellow has a hue angle of 90°, pure green has a hue angle of 180°, and pure blue has a hue angle of 270°. The C* coordinate represents chroma and is specified as a radial distance from the L* axis. C* provides a scale from achromatic, i.e., neutral white, gray, or black, at the L* axis (0 C* units) to greater purity of hue as the coordinate moves away from the L* axis (up to 100 or more C* units). C* and h° can be calculated from a* and b* using Equations 1 and 2:

C*=(a*²+b*²)^{0.5} (1)

h°=arctan(b*/a*) (2)

“Delta E,” “ΔE_{ab}*,” or “ΔE” is a measure of the magnitude of total color difference between two colors represented in CIELAB L*a*b* color space. It has been reported that an experienced color observer cannot distinguish any difference between two colors when the ΔE is about 2.3 or less. The ΔE of two different colors with L*a*b* values, L*₁a*₁b*₁ and L*₂a*₂b*₂, is calculated using Equation 3:

ΔE_{ab}*=\sqrt{(L*₁−L*₂)²+(a*₁−a*₂)²+(b*₁−b*₂)²} (3)

The CIELAB L*a*b* and CIELCH L*C*h° values of FD&C Blue No. 1 at seven different concentrations in aqueous solution are presented in Table 4.

TABLE 4
Aqueous solutions

| Concentration | L* | a* | b* | C* | h° |
|---|---|---|---|---|---|
| 1000 ppm | 10.49 | 15.82 | −44.99 | 47.69 | 289.37 |
| 500 ppm | 24.07 | 9.80 | −58.18 | 59.00 | 279.56 |
| 100 ppm | 52.43 | −29.57 | −57.38 | 64.55 | 242.74 |
| 50 ppm | 63.64 | −43.71 | −48.31 | 65.14 | 227.86 |
| 10 ppm | 84.25 | −37.23 | −23.42 | 43.99 | 212.17 |
| 5 ppm | 90.65 | −24.40 | −14.28 | 28.27 | 210.33 |
| 1 ppm | 97.69 | −6.43 | −3.57 | 7.36 | 209.02 |

The CIELAB L*a*b* and CIELCH L*C*h° values of FD&C Blue No. 1 at six different concentrations on panned candies are presented in Table 5.

TABLE 5
Panned candies

| Concentration | L* | a* | b* | C* | h° |
|---|---|---|---|---|---|
| 0.0075% (75 ppm) | 75.53 | −31.53 | −20.34 | 37.52 | 212.82 |
| 0.01% (100 ppm) | 74.64 | −29.29 | −16.23 | 33.49 | 208.99 |
| 0.02% (200 ppm) | 66.02 | −38.22 | −26.76 | 46.65 | 214.99 |
| 0.04% (400 ppm) | 58.64 | −38.68 | −31.67 | 50.00 | 219.31 |
| 0.05% (500 ppm) | 56.29 | −38.30 | −32.44 | 50.20 | 220.27 |
| 0.1% (1000 ppm) | 47.94 | −31.89 | −36.45 | 48.43 | 228.82 |

These L*a*b* and L*C*h° values for FD&C Blue No. 1 can be used as target values for a non-artificial blue anthocyanin-containing colorant alternative to FD&C Blue No. 1. Non-artificial blue colorants having L*a*b* values that fall within a ΔE of about 2.3 (defined as the just noticeable difference (JND)) or less from these target values would be expected to provide color characteristics sufficiently similar to those provided by FD&C Blue No. 1 that a human eye could not distinguish the difference in color provided by the non-artificial colorant versus the artificial. However, non-artificial blue anthocyanin-containing colorants having L*a*b* values that fall outside a ΔE of about 2.3 can also be used as a non-artificial substitute for FD&C Blue No. 1. The closer the L*a*b* values for a non-artificial blue colorant come to the artificial target values (i.e., yielding smaller values of ΔE), the better replacement the non-artificial blue anthocyanin-containing colorant will be for FD&C Blue No. 1 in an edible application.

Mathematical models can be generated to represent the color characteristics provided by FD&C Blue No. 1 at any concentration in the L*a*b* and L*C*h° color spaces. For example, the color characteristics may be represented by a segmented line model connecting the L*a*b* or L*C*h° data points of Table 3 or 4. A line (L) connecting two points (P₁ and P₂) representing two different concentrations of FD&C Blue No. 1 in L*a*b* space can be calculated with the following Equation 4:

L={P₁+t(P₂−P₁} (4)
wherein, P₁ is (L*₁, a*₁, b*₁); P₂ is (L*₂, a*₂, b*₂); and t is any real number.

Consequently, a segmented line model for FD&C Blue No. 1 in L*a*b* color space can be interpolated based on the L*a*b* values for the seven different concentration points using Equation 4 as follows.

For concentrations between 500 and 1000 ppm, 0<t<1:

L*=10.49+13.58*t

a*=15.82+−6.02*t

b*=−44.99+−13.19*t

For concentrations between 100 and 500 ppm, 0<t<1:

L*=24.07+28.36*t

a*=9.80+−39.37*t

b*=−58.18+0.80*t

For concentrations between 50 and 100 ppm, 0<t<1:

L*=52.43+11.21*t

a*=−29.57+−14.14*t

b*=−57.38+9.07*t

For concentrations between 10 and 50 ppm, 0<t<1:

L*=63.64+20.61*t

a*=−43.71+6.48*t

b*=−48.31+24.89*t

For concentrations between 5 and 10 ppm, 0<t<1:

L*=84.25+6.40*t

a*=−37.23+12.83*t

b*=−23.42+9.14*t

For concentrations between 1 and 5 ppm, 0<t<1:

L*=90.65+7.04*t

a*=−24.40+17.97*t

b*=−14.28+10.71*t

In addition, colors having L*a*b* values falling within a specific ΔE range of the FD&C Blue No. 1 model can be mathematically modeled in L*a*b* color space. Selecting a specific ΔE value, e.g., 15, with respect to FD&C Blue No. 1 and plotting that ΔE in L*a*b* color space results in a tube-like structure around the FD&C Blue No. 1 segmented line model.

To determine whether a point (X₀) in L*a*b* color space falls within a specific ΔE value from the FD&C Blue No. 1 model, the minimum distance, dₘᵢₙ, between the point and the model (represented by line segment X₁ to X₂) must be calculated. Equation 5 can be used to calculate dₘᵢₙ:

$${d}_{min} = \frac{\vert ( {x}_{0} - {x}_{1} ) \times ( {x}_{0} - {x}_{2} ) \vert}{\vert {x}_{2} - {x}_{1} \vert} \qquad ( 5 )$$

wherein × denotes the cross product of two vectors and vertical bars denote the magnitude of a vector expression.

If the value of dₘᵢₙ is less than or equal to the chosen ΔE value, then the point in L*a*b* color space falls within that specific ΔE value from the FD&C Blue No. 1 model.

The colorant compositions disclosed herein include non-artificial blue colorant compositions comprising a fraction of anthocyanins sourced from a non-artificial product, wherein the colorant composition can provide color characteristics having a ΔE value of about 10-12, or less, compared to the color characteristics defined by the segmented line defined by the L*a*b* values of 50 ppm and 100 ppm FD&C Blue No. 1 in aqueous solution. In other embodiments the ΔE value may be less than about 17, about 16, about 15, about 14, about 13, about 12, about 11, about 10, about 9, about 8, about 7, about 6, about 5, about 4, or about 3. The colorant composition may also be measured against a plurality of segmented lines defined by different concentrations of FD&C Blue No. 1 in aqueous solution, e.g., about 1 ppm and about 5 ppm, about 5 ppm and about 10 ppm, about 10 ppm and about 50 ppm, about 100 ppm and about 500 ppm, about 500 ppm and about 1000 ppm, or any combination selected therefrom. However, if ΔE value is used to describe the colorant composition, only one segmented line is required to define the colorant composition.

### 2.6.1 Visible Lambda Max of Compound I

One of the ways in which a colorant is defined is via absorbance in the visible region of the absorption spectrum and the lambda max (λₘₐₓ), which is the wavelength corresponding to the maximal absorbance value of the visible spectra for a solution. Using a λₘₐₓ value and comparison of absorbance over wavelength curves, it is possible to measure, for example, violet contributions.

Other methods can be used to capture color through measurement of solutions in a colorimeter. For example, a Konica Minolta—CM-5 Spectrophotometer can be used in conjunction with appropriate software, for example, color data software CM-S100W and/or SpectraMagic NX. L*a*b*, C, and H (hue angle) values can be obtained for each measurement, and color comparisons can be plotted on an a*b* space (cross section of a spherical color space) at a set value for L*.

In certain embodiments, a solution of the colorant (e.g., Compound I and a metal ion in a solution having a pH of from about 6.0 to about 8.0) will have a measured absorbance of between about 0.5 and about 1.0 in a cuvette having a path length of, e.g., 1 cm, or from about 0.2 mm to about 10 mm, e.g., about 0.2 mm, about 0.3 mm, about 0.5 mm, about 0.7 mm, about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, or about 10 mm, and will have an absorbance maxima (lambda max) of between about 630 nm and about 655 nm, between about 630 and about 650 nm between about 630 nm and about 647 nm, between about 635 and about 647 nm, between about 640 nm and about 647 nm, between about 635 nm and about 655 nm, between about 640 nm and about 655 nm, between about 645 and about 655 nm, between about 650 nm and about 655 nm, or between about 643 nm and about 647 nm. For example, the lambda max value can be determined from a dilute solution of the colorant, which will have an absorbance between about 0.5 and about 1.0 in a cuvette having a path length of about 0.5 cm or about 1 cm. For more concentrated solutions of the colorant, the lambda max value will need to be taken in a cuvette having a shorter path length. The path length of the cuvette is chosen by aiming for an absorbance between about 0.5 to about 1.0 so that the lambda max value can be determined.

### 2.6.2 Calculating the Violet Component of a Blue Colorant

Violet components of a blue colorant can be determined according to the following method. The violet region of the visible light spectrum is defined as the absorbance over the range from about 500 nm to about 600 nm. A sample solution is prepared such that the maximum absorbance in a 1 cm cuvette is about 0.75. For the methods described herein, the cuvette can have a path length of from about 0.2 mm to about 10 mm, e.g., about 0.2 mm, about 0.3 mm, about 0.5 mm, about 0.7 mm, about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, or about 10 mm, depending on the concentration of the solution. The area under the curve of absorbance over wavelength is then calculated for violet contribution. In order to compare data between different spectra, the different spectral data is normalized to allow for direct comparison.

### 2.6.3 Colorimetric Data for Compound I

Colorimetric data for solutions of FD&C Blue No. 1, Compound I with no metal, Compound I with 1.0 mol. eq. of Al³⁺ at pH 7, and Compound I with 1.0 mol. eq. of Al³⁺ at pH 8 are provided below in Table 6, showing λₘₐₓ values; Table 7, showing L*a*b* values; and Table 8, showing violet contribution values.

TABLE 6
λₘₐₓ values

| Material | λₘₐₓ |
|---|---|
| FD&C Blue No. 1 | 630 |
| Compound I with no metal at pH 8 | 607 |
| Compound I + 1.0 mol. eq. Al³⁺ at pH 7 | 636 |
| Compound I + 1.0 mol. eq. Al³⁺ at pH 8 | 637 |

TABLE 7
L*a*b* and ΔE values

| | | | | ΔE (relative |
|---|---|---|---|---|
| Material | L* | a* | b* | to control) |
| FD&C Blue No. 1 solution (control) | 90 | −28 | −15 | — |
| Spirulina solution | 80 | −27 | −29 | 17.15 |
| Compound I + 1.0 mol. eq. Al³⁺ at pH 7 | 83 | −32 | −16 | 8.27 |
| Compound I + no metal at pH 8 | 75 | −20 | −30 | 22.06 |
| Compound I + 1.0 mol. eq. Al³⁺ at pH 8 | 82 | −31 | −18 | 9.18 |

TABLE 8
Violet contribution

| | Violet |
|---|---|
| Material | Contribution |
| FD&C Blue No. 1 | 10.7 |
| Compound I with no metal at pH 8 | 37.5 |
| Compound I + 1.0 mol. eq. Al³⁺ at pH 7 | 20.5 |
| Compound I + 1.0 mol. eq. Al³⁺ at pH 8 | 22.2 |

The L* value for Compound I plus a metal ion (e.g., Al³⁺, Fe²⁺, or Fe³⁺) at about pH 6 to about pH 8 can range from about 70 to about 90, or from about 72 to about 86, or from about 72 to about 80, or from about 73 to about 75. In a specific embodiment, the L* value is about 73.

In certain embodiments, Compound I plus a metal ion (e.g., Al³⁺, Fe²⁺, or Fe³⁺) has a* and b* values (from L*a*b* color space) such that a* ranges from about −20 to about −47, and b* ranges from about −15 to about −30, or more preferably where a* ranges from about −25 to about −35 and b* ranges from about −15 to about −25 or most preferably where a* ranges from about −28 to about −32 and b* ranges from about −15 to about −20.

A computed ΔE value for a solution of Compound I with about 0.3 or about 1.0 mol. equiv. Al³⁺ at about pH 8 or about pH 7 as compared to FD&C Blue No. 1 is less than about 17, wherein FD&C Blue No. 1 has an L* value of about 90. In certain embodiments, the computed ΔE value as compared to FD&C Blue No. 1 is less than about 17, wherein L* is about 83. In certain embodiments, the ΔE is less than about 16, or less than about 15, or less than about 14, or less than about 13, or less than about 12, or less than about 11, or less than about 10, or less than about 9, or less than about 8, or less than about 7, or less than about 6, or less than about 5, or less than about 4, or less than about 3. In certain embodiments, the ΔE can range from about 8 to about 10.

In certain embodiments, the hue angle (from L*C*h° color space) for Compound I at about pH 7 plus a metal ion can range from about 207° to about 230°, or from about 207° to about 225°, or from about 207° to about 220°, or from about 207° to about 215°, or from about 207° to about 211°, or from about 208° to about 210°.

In certain embodiments, the colorant solution has a reduced absorbance and violet contribution in the range of about 500 nm to about 600 nm. In certain embodiments, the absorbance (area under curve) in the range of about 500 nm to about 600 nm is less that about 29, less than about 28, less than about 26, less than about 24, less than about 22 or less than about 21, or less than about 20.

The violet region of visible light spectrum defined as the range from about 500 nm to about 600 nm. The violet color contribution to a blue color is measured by integrating the area under the visible absorbance curve over the wavelength range from about 500 to about 600 nm. The value calculated represents the area under the curve (area units*wavelength) and is a measure of the violet hue present in the blue color. This violet color contribution can be compared for one colorant versus another and in this case will be compared to the violet color for the spectra of a reference solution of FD&C Blue No.1.

### 3. Enzymatic Treatment and Isolation of Anthocyanins

While Compound I with a metal ion (e.g., Al³⁺, Fe²⁺, or Fe³⁺) provides unexpected and desired colorant properties, it has faced difficulties in commercial applications due to the lack of cost-efficient methods of producing Compound I. Compound I is only a minor component of the anthocyanin content in red cabbage juice or extract, as naturally occurring red cabbage juice or extract comprises only from about 1% to about 4% of Compound I. As such, even if Compound I were to be separated from other anthocyanins by fractionation methods or otherwise, large quantities of red cabbage juice would still be necessary to produce Compound I in the quantities required for commercial use. The purification procedures necessary to separate Compound I from the seven other anthocyanin compounds occurring in red cabbage juice also largely increase the costs of the process. Furthermore, there are no known synthetic procedures that would make the compound in commercial quantities. Therefore, new methods for obtaining Compound I are necessary, especially those that would be commercially viable.

As stated previously, structural review of the anthocyanins occurring in red cabbage juice revealed that Compound I is structurally similar to Compounds D-F, with an exception that Compounds D-F have an acyl group (e.g., a para coumaric group, a ferulic group, or a sinapic group) instead of H at the R₂ position of the general anthocyanin compound represented by Formula 1. Hydrolysis of the R₂ ester linkage would result in Compound I. However, a highly selective catalyst is needed for this chemical transformation, as Compound I itself includes an ester group, i.e., a sinapic group at the R₁ position. Specifically, only the acyl group from the 3-glucoside-2″-OH position of anthocyanins (i.e., the R₂ position) should be removed. If both acyl groups (i.e., R₁ and R₂) of Compounds D-F are removed, Compound II, not Compound I, is produced.

The present disclosure utilizes the high specificity of enzymatic catalysis to perform the selective conversion as shown below:

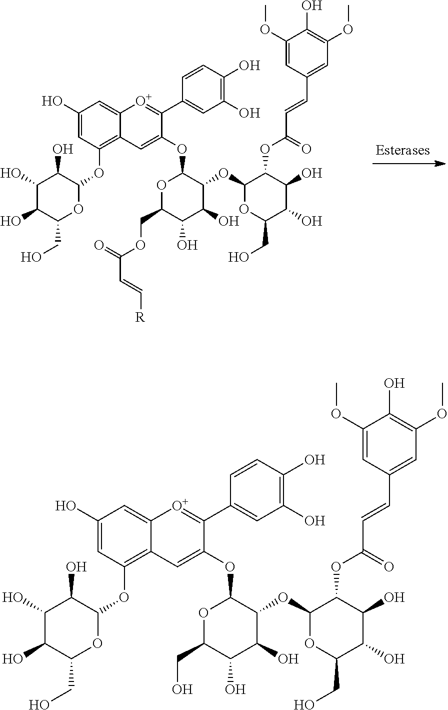

Advantageously, an esterase can be used to enzymatically treat a solution including a mixture of anthocyanins to reduce the total number of anthocyanins in the mixture, i.e., such that only Compound I, or only Compounds I and II, are the remaining anthocyanins of a total anthocyanin content in the solution. This allows for simplified isolation of the desired anthocyanin, e.g., Compound I, from the solution mixture.

### 3.1 Esterases

An esterase refers to a class of enzymes that catalyze the hydrolysis of an ester bond. In certain embodiments, an esterase of the present disclosure catalyzes the hydrolysis of an ester from the 3-glucose of a mono- and/or a di-acylated triglucoside anthocyanin, yielding an anthocyanin with a 3 (2″-carboxyl)-diglucoside-5-glucoside structure. In certain embodiments, an esterase of the present disclosure selectively catalyzes the hydrolysis of an ester from the 3-glucose of a mono- and/or a di-acylated triglucoside anthocyanin without catalyzing the hydrolysis of another ester bond present in the anthocyanin molecule. In certain embodiments, an esterase capable of catalyzing such a reaction is a naturally occurring esterase. In certain other embodiments, an esterase capable of catalyzing such a reaction is a modified esterase. A modified esterase may include at least one amino acid substitution relative to its naturally occurring counterpart.

### 3.1.1 Naturally Occurring Esterases

In certain embodiments, an esterase of the present disclosure comprises an amino acid sequence as set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, or SEQ ID NO: 15. An esterase of the present disclosure (e.g., an esterase of any one of SEQ ID NO: 1-15) can optionally be tagged at the C-terminus with a hexahistidine tag having the amino acid sequence of GGSLEHHHHHH (SEQ ID NO: 90).

In certain embodiments, the esterase of the present disclosure is carboxylesterase from Chromohalobacter salexigens, which has the amino acid sequence set forth in SEQ ID NO: 1, provided below.

[SEQ ID NO: 1]
MADSSPLIIEPRQARAADATVILLHGLGADGHDFE
PLVPALPLAKDLAVRFVLPHAPRMPVTVNGGMEMP
AWYDILDMNLGRRIDEAQLKASADMVHGLIDAEIA
RGIDSRRIIVAGFSQGGAVAYHAALTYPKPLGGLL
ALSTYFATATSIEPSEANRALPIEVHHGSFDPVVP
EALGHEGAERAEALGYAVTYRTYPMQHALCPEQIE
DIGQWLNARLGAKEA.

In certain embodiments, the esterase of the present disclosure is carboxylesterase from Pseudomonas fluorescens, which has the amino acid sequence set forth in SEQ ID NO: 2, provided below.

[SEQ ID NO: 2]
MTEPLILQPAKPADACVIWLHGLGADRYDEMPVAE
ALQESLLTTREVLPQAPTRPVTINGGYEMPSWYDI
KAMSPARSISLEELEVSAKMVTDLIEAQKRTGIDA
SRIFLAGESQGGAVVEHTAFINWQGPLGGVIALST
YAPTEGDELELSASQQRIPALCLHGQYDDVVQNAM
GRSAFEHLKSRGVTVTWQEYPMGHEVLPQEIHDIG
AWLAARLG.

In certain embodiments, the esterase of the present disclosure is carboxylesterase from Pseudomonas aeruginosa, which has the amino acid sequence set forth in SEQ ID NO: 3, provided below.

[SEQ ID NO: 3]
MSEPLILDAPNADACIIWLHGLGADRTDFKPVAEA
LQMVLPSTRFILPQAPSQAVTVNGGWVMPSWYDIL
AFSPARAIDEDQLNASADQVIALIDEQRAKGIAAE
RIILAGFSQGGAVVLHTAFRRYAQPLGGVLALSTY
APTEDDLALDERHKRIPVLHLHGSQDDVVDPALGR
AAHDALQAQGVEVGWHDYPMGHEVSLEEIHDIGAW
LRKRLGGS.

In certain embodiments, the esterase of the present disclosure is arylesterase from Sulfolobos solfataricus, which has the amino acid sequence set forth in SEQ ID NO: 4, provided below.

[SEQ ID NO: 4]
MPLDPEVRNFLQVYYKANIIDFTKYQFQEIRQKVN
ELLAKAVPKDPVGETRDMKIKLEDYELPIRIYSPI
KRTNNGLVMHFHGGAWILGSIETEDAISRILSNSC
ECTVISVDYRLAPEYKFPTAVYDCFNAIVWARDNA
GELGIDKDKIATFGISAGGNLVAATSLLARDNKLK
LTAQVPVVPFVYLDLASKSMNRYRKGYFLDINLPV
DYGVKNYIRDEKDLYNPLFSPLIAEDLSNLPQAIV
VTAEYDPLRDQGEAYAYRLMESGVPTLSFRVNGNV
HAFLGSPRTSRQVTVMIGALLKDIFKGSS.

In certain embodiments, the esterase of the present disclosure is ferulic acid esterase from Aspergillus niger, which has the amino acid sequence set forth in SEQ ID NO: 5, provided below.

[SEQ ID NO: 5]
MKQFSAKYALILLATAGQALAASTQGISEDLYNRL
VEMATISQAAYADLCNIPSTIIKGEKIYNAQTDIN
GWILRDDTSKEIITVERGTGSDTNLQLDTNYTLTP
FDTLPQCNDCEVHGGYYIGWISVQDQVESLVKQQA
SQYPDYALTVTGHSLGASMAALTAAQLSATYDNVR
LYTEGEPRSGNQAFASYMNDAFQVSSPETTQYFRV
THSNDGIPNLPPADEGYAHGGVEYWSVDPYSAQNT
EVCTGDEVQCCEAQGGQGVNDAHTTYFGMTSGACT
W.

In certain embodiments, the esterase of the present disclosure is carboxylesterase from Francisella tularensis, which has the amino acid sequence set forth in SEQ ID NO: 6, provided below.

[SEQ ID NO: 6]
MNYELMEPAKQARFCVIWLHGLGADGHDFVDIVNY
FDVSLDEIRFIFPHADIIPVTINMGMQMRAWYDIK
SLDANSLNRVVDVEGINSSIAKVNKLIDSQVNQGI
ASENIILAGFSQGGIIATYTAITSQRKLGGIMALS
TYLPAWDNFKGKITSINKGLPILVCHGTDDQVLPE
VLGHDLSDKLKVSGFANEYKHYVGMQHSVCMEEIK
DISNFIAKTFKI.

In certain embodiments, the esterase of the present disclosure is alkaline esterase from uncultured bacterium FLS12, which has the amino acid sequence set forth in SEQ ID NO: 7, provided below.

[SEQ ID NO: 7]
MALFQCDFFSDVLGLSTSMTVILPQETTGQIGMAG
GSERREHPTLFLLHGLSDDHTIWLRRTSIERYVAE
MGLAVVMPAVHRSFYTDMAHGLQYWTFISEELPAL
ARSFFPLATAREDTFVAGLSMGGYGALKLGMRHPE
RFAAAASLSGALDITFDPAEHIAMEDDVWVAEQRN
IFGDLAALPGSDHDLFALAERMAQSDGPVPKLYQC
CGTEDFLYEDNVRERDHVRGLGLDEMYEESPGEHE
WGYWDAQIQRVLAWLPLRPPGTAPA.

In certain embodiments, the esterase of the present disclosure is esterase from Pelagibacterium halotolerans, which has the amino acid sequence set forth in SEQ ID NO: 8, provided below.

[SEQ ID NO: 8]
MTEPVKLSGPMLPAVSGAAKSLVVLLHGYGSDGRD
LIALGQFWRDSFPDTMFVAPNAPHVCGGNPFGYEW
FPLDLERDRTLARLAGAETAHPVLDAFLADLWAQT
GLGPADTILVGFSQGAMMALYTGLRLPEPLKAIIA
FSGLIVAPEKLEAEIASKPPVLLIHGDLDDVVPVI
GSETALPKLIDLGIDARLHISQGSGHTIAQDGLDT
ATAFLREIL.

In certain embodiments, the esterase of the present disclosure is lipase carboxylesterase from Sulfolobus islandicus, which has the amino acid sequence set forth in SEQ ID NO: 9, provided below.

[SEQ ID NO: 9]
MMPLDPRIKELLESGFIVPIGKASVDEVRKIFRQL
ASAAPKVEVGKVEDIKIPGSEANINARVYLPKANG
PYGVLIYLHGGGFVIGDVESYDPLCRAITNACNCV
VVSVDYRLAPEYKFPSAVIDSFDATNWVYNNLDKF
DGKMGVAIAGDSAGGNLAAVVALLSKGKLNLKYQI
LIYPAVGFDSVSRSMIEYSDGFFLTREHIEWFGSQ
YLRSPADLLDFRFSPILAQDLSGLPPALIITAEYD
PLRDQGEAYANRLLQAGVPVTSVRFNNVIHGFLSF
FPLIEQGRDAISLIGSVLRRTFYDKS.

In certain embodiments, the esterase of the present disclosure is carboxylesterase from Halomonas sp. QHL1, which has the amino acid sequence set forth in SEQ ID NO: 10, provided below.

[SEQ ID NO: 10]
MTAPGELIIEPKDGQPADACVFIIHGLGADGHDFE
PLVPALALPKDSRVRFIMPHAPRLPVTINGGMVMP
AWYDILAMDLGRRVDERQLKQSAERIQALIQEQID
QGIDSQRIIVAGFSQGGAVAYHAALTFPAPLGGLL
AMSTYFATADNIDLAEANRQIPIEVQHGNFDPIVP
ESLGRSGADRLKEMGYAVNYRQYPMAHALCPQQVN
DIGKWLSARLN.

In certain embodiments, the esterase of the present disclosure is carboxylesterase from Ignatzschineria indica, which has the amino acid sequence set forth in SEQ ID NO: 11, provided below.

[SEQ ID NO: 11]
MDKPIILDPKQSADSAVIWLHGLGATKEDFLPVAQ
ILQRDALPHTRFILPQAPVRPVTLNNGFPMPSWYD
IIALTSPREIKLSELDESSQSIIALIEAEIEKGIP
LERIILAGFSQGGAVVLHTAFIAYPKNVGGVMALS
TYSATFDEAITLDEKKKQIPTLHLHGSLDPVVKIE
LGRAAEQFLKAQGIDTRWHDYPMQHEVINDELQDI
AKWLIERLG.

In certain embodiments, the esterase of the present disclosure is carboxylesterase from Pseudomonas citronellolis, which has the amino acid sequence set forth in SEQ ID NO: 12, provided below.

[SEQ ID NO: 12]
MSQPLLLEPTQPADSCVIWLHGLGADRYDFEPVAR
MLQKVLPRTRFILPQAPTRPVTVFNGMPAPSWYDI
KAMAPARAIDEAQLDASADAVIALIEGQLAEGIAQ
RRIVLAGFSQGGAVVLHTGYLRWPGELGGVMALST
YGPTFDDDLQLPEAKKQQPALCLHGTYDDVVAPAM
GRAAYDFLQRQGVAVQWRDYPMAHEVSNQEIADIA
AWLRERL.

In certain embodiments, the esterase of the present disclosure is beta-lactamase from Myxococcus fulvus, which has the amino acid sequence set forth in SEQ ID NO: 13, provided below.

[SEQ ID NO: 13]
MNGLRWRLTGVVMAWVLVAPLAEAANVKQEVDRYI
SGFHQKGLFNGTVLVANERGILLKKGYGAANLEWK
VPNAPDTKFRIGSITKSFTATVILQLAAEGKLQLD
DPITKHLPDYRKDTGDRVTITHLLNHTSGIPSYTS
KPAIMKDADGFESVAAFVKKACSDDLEFEPGTKYA
YNNSGYFLLGAIIEKLTGQTYAEAVQARILGPLGM
KDTGYDVSATVLPKRASGYAQAPGGIVNAAWLDMN
LPYAAGSLYSTVEDLYRWERAFHGDTLLPAALKQK
MLTPGLAHYGFGWVMSDMTLHDGKTKLPGIFHTGG
INGFSSILVRVPERKEAVILLDNMTHGGLQELAGG
VLSILHGLTPRPARMPIGNVMMESLGKGSVAQATA
TYRTLKKTKQAEYDFSERHLNTVGYHLLRSGRAAD
AIEVFKLNVEMEPEAANCHDSLGEAYAAHGDKARA
ITSYRKALELAPKNEHAVKMLEQLEEPAAKR.

In certain embodiments, the esterase of the present disclosure is carboxylesterase from Pseudomonas saudiphocaensis, which has the amino acid sequence set forth in SEQ ID NO: 14, provided below.

[SEQ ID NO: 14]
MTDPLIIEPAQTADSCVIWLHGLGADRYDEQPVAE
MLQQRLLHTREVLPQAPTRAVTINGGWAMPSWYDI
QAMSPARAIDQAQLEQSAQTVIELIEQQRDSGIDP
RRIFLAGESQGGAVVYHTAFLRWAGPLGGVLALST
YAPTEGDDLKLSPLQAGLPVLCLHGSRDDVVPPAM
GRAAHDCLQQNQVQTQWKEYPMAHEVQPTEIQDIG
DWLASRLG.

In certain embodiments, the esterase of the present disclosure is carboxylesterase from Ventosimonas gracilis, which has the amino acid sequence set forth in SEQ ID NO: 15, provided below.

[SEQ ID NO: 15]
MTEPLIIEPSQPADSAVIWLHGLGADRFDFEPVARLLGQHLPSTRFILPQ
APTRPVTFNMGHAMPSWYDILALDGSERAINPADLEASSETLIALINAQQ
QSGIDSKRIVLAGFSQGGAVVLHTALLRFDEKLAGVLALSTYAPTFNAET
QFAESKQNLPVLCMHGSEDAVLPISMGRAVYDKLSEQGIKANWRDYPMGH
EVRPEQLRDILDWLKNTLPSLP.

### 3.1.2 Modified Esterases

The disclosure also features modified esterases useful for catalyzing the hydrolysis of an ester bond in an anthocyanin. A modified esterase can include modifications that either maintain the activity of the naturally occurring enzyme or enhances it, e.g., by increasing the rate of conversion or by increasing the percent completion of the reaction. Modifications can be made at the active site of the native sequence, or at any other location within the native sequence of the naturally occurring enzyme. In certain embodiments, a modified esterase of the present disclosure includes those containing an active site motif(s) having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity, or having at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acid substitutions relative to the reference sequence, i.e., the naturally occurring counterpart to the modified esterase.

For example, the active site of a naturally occurring enzyme can be the active site of a carboxylesterase from Pseudomonas fluorescens having the amino acid sequence of SEQ ID NO: 2. The active site of the carboxylesterase from P. fluorescens having the amino acid sequence of SEQ ID NO: 2 is comprised of catalytically relevant amino acids at position numbers 25, 28, 29, 30, 31, 38, 39, 63, 65, 70, 73, 200, and 201. In certain embodiments, a modified esterase of the present disclosure is based on a scaffold of a carboxylesterase from P. fluorescens and includes an amino acid substitution at one or more amino acid position numbers of 25, 28, 29, 30, 31, 38, 39, 63, 65, 70, 73, 200, and 201 of the carboxylesterase from P. fluorescens. An amino acid substitution in a modified esterase relative to its naturally occurring counterpart can be a conservative or a nonconservative amino acid substitution as is known and understood in the art. For example, in certain embodiments, a modified esterase of the present disclosure is based on the carboxylesterase from P. fluorescens of SEQ ID NO: 2, and includes an amino acid substitution at amino acid position number 73 of SEQ ID NO: 2. The amino acid substitution at amino acid position number 73 can be, for instance, a methionine to histidine amino acid substitution. In one particular example, a modified esterase of the present disclosure comprises the amino acid sequence of SEQ ID NO: 16.

In another example, a modified esterase of the present disclosure is based on a scaffold of a carboxylesterase from Chromohalobacter salexigens having the amino acid sequence of SEQ ID NO: 1. The active site of the carboxylesterase from C. salexigens having the amino acid sequence of SEQ ID NO: 1 can be comprised of catalytically relevant amino acids at position numbers 23, 28, 75, 115, 117, 119, 121, 122, 125, 129, 168, 171, 173, 202, 209, and/or 212. In certain embodiments, a modified esterase of the present disclosure is based on a scaffold of a carboxylesterase from C. salexigens and includes an amino acid substitution at one or more amino acid position numbers of 23, 28, 75, 115, 117, 119, 121, 122, 125, 129, 168, 171, 173, 202, 209, or 212. An amino acid substitution in a modified esterase relative to its naturally occurring counterpart can be a conservative or a nonconservative amino acid substitution as is known and understood in the art. In other nonlimiting embodiments, the active site of a carboxylesterase of C. salexigens having the amino acid sequence of SEQ ID NO: 1 can be modified to include a I, V, or L at amino acid position 23; A, S, or T at amino acid position 28; I, V, or L at amino acid position 75; I, V, or L at amino acid position 115; A, G, P, or S (tiny residue) at amino acid position 116; G at amino acid position 117; S at amino acid position 119; G at amino acid position 121; G at amino acid position 122; V or A at amino acid position 125; A, G, or S at amino acid position 129; A, G, or S at amino acid position 168; D at amino acid position 171; I, V, or L at amino acid position 173; H at amino acid position 203; I, V, or L at amino acid position 209; and/or I, V, or L at amino acid position 212.

In some embodiments, the modified esterase of the present disclosure comprises an amino acid sequence having at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acid substitutions relative to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, or SEQ ID NO: 15. For instance, a modified esterase can include an amino acid substitution at, for example, an amino acid at position 25, 28, 29, 30, 31, 38, 39, 63, 65, 70, 73, 200, and/or 201 of SEQ ID NO: 2. In another example, a modified esterase can include an amino acid substitution at, for example, an amino acid at position 23, 28, 75, 115, 117, 119, 121, 122, 125, 129, 168, 171, 173, 202, 209, and/or 212 of SEQ ID NO: 1. Such an amino acid substitution can be a conservative or a nonconservative amino acid substitution as known in the art. In certain non-limiting embodiments, the modified esterase maintains the original active site motif of its naturally occurring counterpart. In other embodiments, the modified esterase maintains an active site having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity relative to the original active site motif of its naturally occurring counterpart, e.g., wherein the naturally occurring counterpart is an enzyme having the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, or SEQ ID NO: 15.

For example, in certain embodiments, the esterase of the present disclosure is a modified esterase comprising an amino acid sequence set forth in SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, or SEQ ID NO: 43. A modified esterase of the present disclosure (e.g., a modified esterase of any one of SEQ ID NO: 16-43) can optionally be tagged at the C-terminus with a hexahistidine tag having the sequence of SEQ ID NO: 90.

In certain embodiments, the esterase of the present disclosure is carboxylesterase from Pseudomonas fluorescens modified by an amino acid substitution at position 73, which has the amino acid sequence set forth in SEQ ID NO: 16, provided below.

[SEQ ID NO: 16]
MTEPLILQPAKPADACVIWLHGLGADRYDFMPVAEALQESLLTTRFVLPQ
APTRPVTINGGYEMPSWYDIKAHSPARSISLEELEVSAKMVTDLIEAQKR
TGIDASRIFLAGFSQGGAVVFHTAFINWQGPLGGVIALSTYAPTFGDELE
LSASQQRIPALCLHGQYDDVVQNAMGRSAFEHLKSRGVTVTWQEYPMGHE
VLPQEIHDIGAWLAARLG.

In certain embodiments, the esterase of the present disclosure is carboxylesterase from Pseudomonas fluorescens modified by an amino acid substitution at position 29, which has the amino acid sequence set forth in SEQ ID NO: 17, provided below.

[SEQ ID NO: 17]
MTEPLILQPAKPADACVIWLHGLGADRYGFMPVAEALQESLLTTRFVLPQ
APTRPVTINGGYEMPSWYDIKAMSPARSISLEELEVSAKMVTDLIEAQKR
TGIDASRIFLAGFSQGGAVVFHTAFINWQGPLGGVIALSTYAPTFGDELE
LSASQQRIPALCLHGQYDDVVQNAMGRSAFEHLKSRGVTVTWQEYPMGHE
VLPQEIHDIGAWLAARLG.

In certain embodiments, the esterase of the present disclosure is carboxylesterase from Pseudomonas fluorescens modified by an amino acid substitution at position 73, which has the amino acid sequence set forth in SEQ ID NO: 18, provided below.

[SEQ ID NO: 18]
MTEPLILQPAKPADACVIWLHGLGADRYDFMPVAEALQESLLTTRFVLPQ
APTRPVTINGGYEMPSWYDIKAVSPARSISLEELEVSAKMVTDLIEAQKR
TGIDASRIFLAGFSQGGAVVFHTAFINWQGPLGGVIALSTYAPTFGDELE
LSASQQRIPALCLHGQYDDVVQNAMGRSAFEHLKSRGVTVTWQEYPMGHE
VLPQEIHDIGAWLAARLG.

In certain embodiments, the esterase of the present disclosure is carboxylesterase from Pseudomonas fluorescens modified by an amino acid substitution at position 200, which has the amino acid sequence set forth in SEQ ID NO: 19, provided below.

[SEQ ID NO: 19]
MTEPLILQPAKPADACVIWLHGLGADRYDFMPVAEALQESLLTTRFVLPQ
APTRPVTINGGYEMPSWYDIKAMSPARSISLEELEVSAKMVTDLIEAQKR
TGIDASRIFLAGFSQGGAVVFHTAFINWQGPLGGVIALSTYAPTFGDELE
LSASQQRIPALCLHGQYDDVVQNAMGRSAFEHLKSRGVTVTWQEYPMGHS
VLPQEIHDIGAWLAARLG.

In certain embodiments, the esterase of the present disclosure is carboxylesterase from Pseudomonas fluorescens modified by an amino acid substitution at position 200, which has the amino acid sequence set forth in SEQ ID NO: 20, provided below.

[SEQ ID NO: 20]
MTEPLILQPAKPADACVIWLHGLGADRYDFMPVAEALQESLLTTRFVLPQ
APTRPVTINGGYEMPSWYDIKAMSPARSISLEELEVSAKMVTDLIEAQKR
TGIDASRIFLAGFSQGGAVVFHTAFINWQGPLGGVIALSTYAPTFGDELE
LSASQQRIPALCLHGQYDDVVQNAMGRSAFEHLKSRGVTVTWQEYPMGHT
VLPQEIHDIGAWLAARLG.

In certain embodiments, the esterase of the present disclosure is carboxylesterase from Pseudomonas fluorescens modified by an amino acid substitution at positions 65 and 73, which has the amino acid sequence set forth in SEQ ID NO: 21, provided below.

[SEQ ID NO: 21]
MTEPLILQPAKPADACVIWLHGLGADRYDFMPVAEALQESLLTTRFVLPQ
APTRPVTINGGYEMSSWYDIKAHSPARSISLEELEVSAKMVTDLIEAQKR
TGIDASRIFLAGFSQGGAVVFHTAFINWQGPLGGVIALSTYAPTFGDELE
LSASQQRIPALCLHGQYDDVVQNAMGRSAFEHLKSRGVTVTWQEYPMGHE
VLPQEIHDIGAWLAARLG.

In certain embodiments, the esterase of the present disclosure is carboxylesterase from Pseudomonas fluorescens modified by an amino acid substitution at position 63, which has the amino acid sequence set forth in SEQ ID NO: 22, provided below.

[SEQ ID NO: 22]
MTEPLILQPAKPADACVIWLHGLGADRYDFMPVAEALQESLLTTRFVLPQ
APTRPVTINGGYDMPSWYDIKAMSPARSISLEELEVSAKMVTDLIEAQKR
TGIDASRIFLAGFSQGGAVVFHTAFINWQGPLGGVIALSTYAPTFGDELE
LSASQQRIPALCLHGQYDDVVQNAMGRSAFEHLKSRGVTVTWQEYPMGHE
VLPQEIHDIGAWLAARLG.

In certain embodiments, the esterase of the present disclosure is carboxylesterase from Pseudomonas fluorescens modified by an amino acid substitution at positions 25, 70, and 73, which has the amino acid sequence set forth in SEQ ID NO: 23, provided below.

[SEQ ID NO: 23]
MTEPLILQPAKPADACVIWLHGLGGDRYDFMPVAEALQESLLTTRFVLPQ
APTRPVTINGGYEMPSWYDLKAVSPARSISLEELEVSAKMVTDLIEAQKR
TGIDASRIFLAGFSQGGAVVFHTAFINWQGPLGGVIALSTYAPTFGDELE
LSASQQRIPALCLHGQYDDVVQNAMGRSAFEHLKSRGVTVTWQEYPMGHE
VLPQEIHDIGAWLAARLG.

In certain embodiments, the esterase of the present disclosure is carboxylesterase from Pseudomonas fluorescens modified by an amino acid substitution at positions 70, 73, and 200, which has the amino acid sequence set forth in SEQ ID NO: 24, provided below.

[SEQ ID NO: 24]
MTEPLILQPAKPADACVIWLHGLGADRYDFMPVAEALQESLLTTRFVLPQ
APTRPVTINGGYEMPSWYDLKAVSPARSISLEELEVSAKMVTDLIEAQKR
TGIDASRIFLAGFSQGGAVVFHTAFINWQGPLGGVIALSTYAPTFGDELE
LSASQQRIPALCLHGQYDDVVQNAMGRSAFEHLKSRGVTVTWQEYPMGHS
VLPQEIHDIGAWLAARLG.

In certain embodiments, the esterase of the present disclosure is carboxylesterase from Pseudomonas fluorescens modified by an amino acid substitution at positions 200, and 201, which has the amino acid sequence set forth in SEQ ID NO: 25, provided below.

[SEQ ID NO: 25]
MTEPLILQPAKPADACVIWLHGLGADRYDFMPVAEALQESLLTTRFVLPQ
APTRPVTINGGYEMPSWYDIKAMSPARSISLEELEVSAKMVTDLIEAQKR
TGIDASRIFLAGFSQGGAVVFHTAFINWQGPLGGVIALSTYAPTFGDELE
LSASQQRIPALCLHGQYDDVVQNAMGRSAFEHLKSRGVTVTWQEYPMGHT
ILPQEIHDIGAWLAARLG.

In certain embodiments, the esterase of the present disclosure is carboxylesterase from Pseudomonas fluorescens modified by an amino acid substitution at positions 73, and 200, which has the amino acid sequence set forth in SEQ ID NO: 26, provided below.

[SEQ ID NO: 26]
MTEPLILQPAKPADACVIWLHGLGADRYDFMPVAEALQESLLTTRFVLPQ
APTRPVTINGGYEMPSWYDIKAVSPARSISLEELEVSAKMVTDLIEAQKR
TGIDASRIFLAGFSQGGAVVFHTAFINWQGPLGGVIALSTYAPTFGDELE
LSASQQRIPALCLHGQYDDVVQNAMGRSAFEHLKSRGVTVTWQEYPMGHS
VLPQEIHDIGAWLAARLG.

In certain embodiments, the esterase of the present disclosure is carboxylesterase from Pseudomonas fluorescens modified by an amino acid substitution at positions 25, 28, 70, and 73, which has the amino acid sequence set forth in SEQ ID NO: 27, provided below.

[SEQ ID NO: 27]
MTEPLILQPAKPADACVIWLHGLGAGRTDFMPVAEALQESLLTTRFVLPQ
APTRPVTINGGYEMPSWYDLKAVSPARSISLEELEVSAKMVTDLIEAQKR
TGIDASRIFLAGFSQGGAVVFHTAFINWQGPLGGVIALSTYAPTFGDELE
LSASQQRIPALCLHGQYDDVVQNAMGRSAFEHLKSRGVTVTWQEYPMGHE
VLPQEIHDIGAWLAARLG.

In certain embodiments, the esterase of the present disclosure is carboxylesterase from Pseudomonas fluorescens modified by an amino acid substitution at positions 25, 28, 29, 30, and 31, which has the amino acid sequence set forth in SEQ ID NO: 28, provided below.

[SEQ ID NO: 28]
MTEPLILQPAKPADACVIWLHGLGAGRTSHRPVAEALQESLLTTRFVLPQ
APTRPVTINGGYEMPSWYDIKAMSPARSISLEELEVSAKMVTDLIEAQKR
TGIDASRIFLAGFSQGGAVVFHTAFINWQGPLGGVIALSTYAPTFGDELE
LSASQQRIPALCLHGQYDDVVQNAMGRSAFEHLKSRGVTVTWQEYPMGHE
VLPQEIHDIGAWLAARLG.

In certain embodiments, the esterase of the present disclosure is carboxylesterase from Pseudomonas fluorescens modified by an amino acid substitution at position 31, which has the amino acid sequence set forth in SEQ ID NO: 29, provided below.

[SEQ ID NO: 29]
MTEPLILQPAKPADACVIWLHGLGADRYDFKPVAEALQESLLTTRFVLPQ
APTRPVTINGGYEMPSWYDIKAMSPARSISLEELEVSAKMVTDLIEAQKR
TGIDASRIFLAGFSQGGAVVFHTAFINWQGPLGGVIALSTYAPTFGDELE
LSASQQRIPALCLHGQYDDVVQNAMGRSAFEHLKSRGVTVTWQEYPMGHE
VLPQEIHDIGAWLAARLG.

In certain embodiments, the esterase of the present disclosure is carboxylesterase from Pseudomonas fluorescens modified by an amino acid substitution at position 31, which has the amino acid sequence set forth in SEQ ID NO: 30, provided below.

[SEQ ID NO: 30]
MTEPLILQPAKPADACVIWLHGLGADRYDERPVAEALQESLLTTRFVLP
QAPTRPVTINGGYEMPSWYDIKAMSPARSISLEELEVSAKMVTDLIEAQ
KRTGIDASRIFLAGESQGGAVVFHTAFINWQGPLGGVIALSTYAPTFGD
ELELSASQQRIPALCLHGQYDDVVQNAMGRSAFEHLKSRGVTVTWQEYP
MGHEVLPQEIHDIGAWLAARLG.

In certain embodiments, the esterase of the present disclosure is carboxylesterase from Pseudomonas fluorescens modified by an amino acid substitution at position 70, which has the amino acid sequence set forth in SEQ ID NO: 31, provided below.

[SEQ ID NO: 31]
MTEPLILQPAKPADACVIWLHGLGADRYDEMPVAEALQESLLTTRFVLP
QAPTRPVTINGGYEMPSWYDLKAMSPARSISLEELEVSAKMVTDLIEAQ
KRTGIDASRIFLAGESQGGAVVFHTAFINWQGPLGGVIALSTYAPTFGD
ELELSASQQRIPALCLHGQYDDVVQNAMGRSAFEHLKSRGVTVTWQEYP
MGHEVLPQEIHDIGAWLAARLG.

In certain embodiments, the esterase of the present disclosure is carboxylesterase from Pseudomonas fluorescens modified by an amino acid substitution at position 73, which has the amino acid sequence set forth in SEQ ID NO: 32, provided below.

[SEQ ID NO: 32]
MTEPLILQPAKPADACVIWLHGLGADRYDFMPVAEALQESLLTTRFVLP
QAPTRPVTINGGYEMPSWYDIKALSPARSISLEELEVSAKMVTDLIEAQ
KRTGIDASRIFLAGESQGGAVVFHTAFINWQGPLGGVIALSTYAPTFGD
ELELSASQQRIPALCLHGQYDDVVQNAMGRSAFEHLKSRGVTVTWQEYP
MGHEVLPQEIHDIGAWLAARLG.

In certain embodiments, the esterase of the present disclosure is carboxylesterase from Pseudomonas fluorescens modified by an amino acid substitution at positions 28, and 73, which has the amino acid sequence set forth in SEQ ID NO: 33, provided below.

[SEQ ID NO: 33]
MTEPLILQPAKPADACVIWLHGLGADRTDFMPVAEALQESLLTTREVLP
QAPTRPVTINGGYEMPSWYDIKAVSPARSISLEELEVSAKMVTDLIEAQ
KRTGIDASRIFLAGFSQGGAVVFHTAFINWQGPLGGVIALSTYAPTFGD
ELELSASQQRIPALCLHGQYDDVVQNAMGRSAFEHLKSRGVTVTWQEYP
MGHEVLPQEIHDIGAWLAARLG.

In certain embodiments, the esterase of the present disclosure is carboxylesterase from Pseudomonas fluorescens modified by an amino acid substitution at positions 25, and 73, which has the amino acid sequence set forth in SEQ ID NO: 34, provided below.

[SEQ ID NO: 34]
MTEPLILQPAKPADACVIWLHGLGAGRYDFMPVAEALQESLLTTRFVLP
QAPTRPVTINGGYEMPSWYDIKAVSPARSISLEELEVSAKMVTDLIEAQ
KRTGIDASRIFLAGESQGGAVVFHTAFINWQGPLGGVIALSTYAPTFGD
ELELSASQQRIPALCLHGQYDDVVQNAMGRSAFEHLKSRGVTVTWQEYP
MGHEVLPQEIHDIGAWLAARLG.

In certain embodiments, the esterase of the present disclosure is carboxylesterase from Pseudomonas fluorescens modified by an amino acid substitution at positions 25, and 28, which has the amino acid sequence set forth in SEQ ID NO: 35, provided below.

[SEQ ID NO: 35]
MTEPLILQPAKPADACVIWLHGLGGDRTDFMPVAEALQESLLTTRFVLP
QAPTRPVTINGGYEMPSWYDIKAMSPARSISLEELEVSAKMVTDLIEAQ
KRTGIDASRIFLAGESQGGAVVFHTAFINWQGPLGGVIALSTYAPTFGD
ELELSASQQRIPALCLHGQYDDVVQNAMGRSAFEHLKSRGVTVTWQEYP
MGHEVLPQEIHDIGAWLAARLG.

In certain embodiments, the esterase of the present disclosure is carboxylesterase from Pseudomonas fluorescens modified by an amino acid substitution at positions 70, and 73, which has the amino acid sequence set forth in SEQ ID NO: 36, provided below.

[SEQ ID NO: 36]
MTEPLILQPAKPADACVIWLHGLGADRYDFMPVAEALQESLLTTREVLP
QAPTRPVTINGGYEMPSWYDLKAASPARSISLEELEVSAKMVTDLIEAQ
KRTGIDASRIFLAGESQGGAVVFHTAFINWQGPLGGVIALSTYAPTFGD
ELELSASQQRIPALCLHGQYDDVVQNAMGRSAFEHLKSRGVTVTWQEYP
MGHEVLPQEIHDIGAWLAARLG.

In certain embodiments, the esterase of the present disclosure is carboxylesterase from Pseudomonas fluorescens modified by an amino acid substitution at positions 70, and 73, which has the amino acid sequence set forth in SEQ ID NO: 37, provided below.

[SEQ ID NO: 37]
MTEPLILQPAKPADACVIWLHGLGADRYDFMPVAEALQESLLTTRFVLP
QAPTRPVTINGGYEMPSWYDLKALSPARSISLEELEVSAKMVTDLIEAQ
KRTGIDASRIFLAGESQGGAVVFHTAFINWQGPLGGVIALSTYAPTFGD
ELELSASQQRIPALCLHGQYDDVVQNAMGRSAFEHLKSRGVTVTWQEYP
MGHEVLPQEIHDIGAWLAARLG.

In certain embodiments, the esterase of the present disclosure is carboxylesterase from Pseudomonas fluorescens modified by an amino acid substitution at position 30, which has the amino acid sequence set forth in SEQ ID NO: 38, provided below.

[SEQ ID NO: 38]
MTEPLILQPAKPADACVIWLHGLGADRYDHMPVAEALQESLLTTRFVLP
QAPTRPVTINGGYEMPSWYDIKAMSPARSISLEELEVSAKMVTDLIEAQ
KRTGIDASRIFLAGESQGGAVVFHTAFINWQGPLGGVIALSTYAPTFGD
ELELSASQQRIPALCLHGQYDDVVQNAMGRSAFEHLKSRGVTVTWQEYP
MGHEVLPQEIHDIGAWLAARLG.

In certain embodiments, the esterase of the present disclosure is carboxylesterase from Pseudomonas fluorescens modified by an amino acid substitution at positions 138, and 139, which has the amino acid sequence set forth in SEQ ID NO: 39, provided below.

[SEQ ID NO: 39]
MTEPLILQPAKPADACVIWLHGLGADRYDFMPVAEALQESLLTTRFVLP
QAPTRPVTINGGYEMPSWYDIKAMSPARSISLEELEVSAKMVTDLIEAQ
KRTGIDASRIFLAGESQGGAVVFHTAFINWQGPLGGVIAVNTYAPTFGD
ELELSASQQRIPALCLHGQYDDVVQNAMGRSAFEHLKSRGVTVTWQEYP
MGHEVLPQEIHDIGAWLAARLG.

In certain embodiments, the esterase of the present disclosure is carboxylesterase from Pseudomonas fluorescens modified by an amino acid substitution at position 25, which has the amino acid sequence set forth in SEQ ID NO: 40, provided below.

[SEQ ID NO: 40]
MTEPLILQPAKPADACVIWLHGLGGDRYDFMPVAEALQESLLTTRFVLP
QAPTRPVTINGGYEMPSWYDIKAMSPARSISLEELEVSAKMVTDLIEAQ
KRTGIDASRIFLAGESQGGAVVFHTAFINWQGPLGGVIALSTYAPTFGD
ELELSASQQRIPALCLHGQYDDVVQNAMGRSAFEHLKSRGVTVTWQEYP
MGHEVLPQEIHDIGAWLAARLG.

In certain embodiments, the esterase of the present disclosure is carboxylesterase from Pseudomonas fluorescens modified by an amino acid substitution at positions 70, and 73, which has the amino acid sequence set forth in SEQ ID NO: 41, provided below.

[SEQ ID NO: 41]
MTEPLILQPAKPADACVIWLHGLGADRYDEMPVAEALQESLLTTRFVLP
QAPTRPVTINGGYEMPSWYDLKAVSPARSISLEELEVSAKMVTDLIEAQ
KRTGIDASRIFLAGESQGGAVVFHTAFINWQGPLGGVIALSTYAPTFGD
ELELSASQQRIPALCLHGQYDDVVQNAMGRSAFEHLKSRGVTVTWQEYP
MGHEVLPQEIHDIGAWLAARLG.

In certain embodiments, the esterase of the present disclosure is carboxylesterase from Pseudomonas fluorescens modified by an amino acid substitution at positions 70, and 73, which has the amino acid sequence set forth in SEQ ID NO: 42, provided below.

[SEQ ID NO: 42]
MTEPLILQPAKPADACVIWLHGLGADRYDFMPVAEALQESLLTTRFVLP
QAPTRPVTINGGYEMPSWYDLKASSPARSISLEELEVSAKMVTDLIEAQ
KRTGIDASRIFLAGESQGGAVVFHTAFINWQGPLGGVIALSTYAPTFGD
ELELSASQQRIPALCLHGQYDDVVQNAMGRSAFEHLKSRGVTVTWQEYP
MGHEVLPQEIHDIGAWLAARLG.

In certain embodiments, the esterase of the present disclosure is carboxylesterase from Pseudomonas fluorescens modified by an amino acid substitution at positions 25, 28, 29, 30, 31, 200, and 201, which has the amino acid sequence set forth in SEQ ID NO: 43, provided below.

[SEQ ID NO: 43]
MTEPLILQPAKPADACVIWLHGLGAGRTSHRPVAEALQESLLTTRFVLP
QAPTRPVTINGGYEMPSWYDIKAMSPARSISLEELEVSAKMVTDLIEAQ
KRTGIDASRIFLAGESQGGAVVFHTAFINWQGPLGGVIALSTYAPTFGD
ELELSASQQRIPALCLHGQYDDVVQNAMGRSAFEHLKSRGVTVTWQEYP
MGHTILPQEIHDIGAWLAARLG.

In certain embodiments, the present disclosure further includes a nucleic acid sequence (e.g., a DNA sequence or an RNA sequence, optionally comprised within a vector) encoding a modified esterase as disclosed herein, such as the modified esterase of any one of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, and SEQ ID NO: 43. Such a nucleic acid sequence may optionally be codon optimized for expression in a host organism.

### 3.2 Enzymatic Ester Hydrolysis to Obtain Compound I

To synthesize Compound I, an enzyme of any one of SEQ ID NO: 1-43 can be used to enzymatically treat a solution including an anthocyanin or a mixture of anthocyanins (i.e., a mixture of a mono- and/or a diacylated anthocyanins), such that the anthocyanin compounds in the solution are selectively deacylated, resulting in either Compound I or II as the product. The solution can be, e.g., a fruit of vegetable extract, such as red cabbage juice or extract. The anthocyanins in the solution can be, e.g., an anthocyanin compound of any one of Compounds A-F, I, and II.

In certain embodiments, the enzymatic treatment of a solution (e.g., red cabbage juice or extract) by contacting the solution with an esterase of any one of SEQ ID NO: 1-43 results in the increase of the amount of Compound I in the solution by about 1-fold, about 10-fold, about 20-fold, about 30-fold, about 40-fold, about 50-fold, about 60-fold, about 70-fold, about 80-fold, about 90-fold, or about 100-fold relative to the amount of Compound I occurring in the solution (e.g., red cabbage juice or extract) prior to the enzymatic treatment. In certain embodiments, following enzymatic treatment of the solution, Compound I is present in the solution in an amount of about 40%, about 45%, about 50%, about 55%, about 60%, about 65%, about 70%, about 75%, about 80%, about 85%, about 90%, about 95%, about 98%, or about 100% of the total anthocyanin content of the solution. In other embodiments, the solution further comprises an amount of Compound II, which can be separated from Compound I according to the procedures disclosed herein.

The ester hydrolysis of an anthocyanin to Compound I can be catalyzed by a solution including one particular enzyme of SEQ ID NO: 1-43, or a mixture of enzymes selected from one of SEQ ID NO: 1-43. In certain embodiments, one or more enzymes of SEQ ID NO: 1-43 can be used to convert one or more diacylated anthocyanins in red cabbage juice to Compound I. In certain embodiments, one enzyme of SEQ ID NO: 1-43 is used to convert one or more diacylated anthocyanins in red cabbage juice to Compound I. In certain other embodiments, two enzymes of SEQ ID NO: 1-43 are used to convert one or more diacylated anthocyanins in red cabbage juice to Compound I. In certain other embodiments, three enzymes of SEQ ID NO: 1-43 are used to convert one or more diacylated anthocyanins in red cabbage juice to Compound I. In certain other embodiments, four or more enzymes of SEQ ID NO: 1-43 are used to convert one or more diacylated anthocyanins in red cabbage juice to Compound I.

The enzymatic ester hydrolysis of the solution can occur in a buffer solution having a pH of about 6.5-8 at a temperature of about 18-35° C. The amount of the enzyme of SEQ ID NO: 1-43 used for the enzymatic ester hydrolysis can be, for example, at least about 0.25 mg (e.g., at least about 0.30 mg, at least about 0.35 mg, at least about 0.40 mg, at least about 0.45 mg, at least about 0.50 mg, at least about 0.55 mg, at least about 0.60 mg, at least about 0.65 mg, at least about 0.70 mg, at least about 0.75 mg, at least about 0.80 mg, at least about 0.85 mg, at least about 0.90 mg, at least about 1.0 mg, at least about 1.5 mg, at least about 2.0 mg) enzyme per about 10 mg of substrate (e.g., a solution containing an anthocyanin, such as red cabbage juice or extract). An exemplary reaction is described in Example 1; however, one of ordinary skill in the art would recognize that reaction conditions other than those described below can be used for the enzymatic ester hydrolysis described herein, and would be able to determine the appropriate conditions for the reaction.

In certain embodiments, the one or more enzymes of SEQ ID NO: 1-43 catalyzes the hydrolysis of a compound of any one of Compounds A-C in a solution, e.g., of red cabbage juice or extract, to produce Compound II. In certain embodiments, the enzymatic treatment of a solution, e.g., red cabbage juice or extract, results in a solution where Compounds I and II make up the major anthocyanin content of the solution. In certain embodiments, Compounds I and II is present in an amount of at least about 40%, about 50%, about 60%, about 70%, about 80%, about 90%, about 95%, about 96%, about 97%, about 98%, about 99%, or about 100% of the total anthocyanin content of the solution, e.g., the red cabbage juice, that has undergone the enzymatic treatment disclosed herein. In certain embodiments, after the enzymatic treatment, Compound I is present in the solution in an amount of about 40%, about 45%, about 50%, about 55%, about 60%, about 65%, about 70%, about 75%, about 80%, about 85%, about 90%, about 95%, about 96%, about 97%, about 98%, about 99%, or about 100%, or from about 40% to about 100%, from about 40% to about 60%, from about 40% to about 80%, from about 60% to about 80%, from about 60% to about 100%, or from about 80% to about 100% by weight of the solution.

### 3.3 Isolation of Anthocyanins

In certain embodiments, the enzymatic treatment of fruit and/or vegetable juice and/or extract described herein provides a mixture of one or more anthocyanins, and further purification is necessary to isolate particular anthocyanins of interest. In certain embodiments, the purification process includes one or more of enzyme precipitation, solid-phase extraction, and preparatory HPLC. In some embodiments, the purification process includes (i) enzyme precipitation, followed by (ii) solid-phase extraction, optionally followed by (iii) preparatory HPLC.

In certain embodiments, the enzyme precipitation step comprises adjusting the pH of the mixture. In certain embodiments, the pH of the mixture is adjusted to a low pH of from about 1 to about 2.5, from about 1 to about 1.5, from about 1.25 to about 1.75, from about 1.5 to about 2, from about 1.75 to about 2.25, or from about 2 to about 2.5. In a particular embodiment the pH of the mixture is adjust to pH of about 1.10. In certain embodiments, a strong inorganic acid is used to adjust pH of the mixture. In certain embodiments, the acid can be hydrochloric acid, sulfuric acid, or orthophosphoric acid. In certain embodiments, after lowering pH of the mixture, the mixture is cooled in an ice bath for a time period of from about 1.5 hours to about 2.5 hours and the liquid can then be transferred to centrifuge tubes and centrifuged. In certain embodiments, the mixture is centrifuged for from about 2 to about 10 minutes. In a particular embodiment, the mixture is centrifuged for about 5 minutes. In certain embodiments, the mixture is centrifuged at from about 3000 rcf to about 4000 rcf. In a particular embodiment, the mixture is centrifuged at about 3540 rcf for about 5 minutes.

In certain embodiments, after the centrifugation step, the supernatant is decanted and subsequently vacuum filtered. In certain embodiments, the resulting material is further purified by using Solid Phase Extraction.

In certain embodiments, the mixture contains two or more anthocyanins that need to be separated from each other. In certain embodiments, the anthocyanins are separated by using High Performance Liquid Chromatography (HPLC). In certain embodiments, the HPLC system is equipped with a diode array detector (DAD). In certain embodiments, ethanol must be removed from the mixture prior to preparatory HPLC-DAD. In certain embodiments, the ethanol is removed by placing the mixture in a warm water bath at a temperature of from about 25° C. to about 45° C. with a steady stream of an inert gas such as nitrogen, argon, or helium passing over. In certain embodiments, deionized water is added to the mixture to account for the volume of ethanol removed.

LC columns of various length, diameter, particle size and pore size can be used to separate two or more anthocyanins, depending on the identity of the anthocyanin and the amount of the solution. In a specific embodiment, a 250 mm L×50 mm D Phenomenex Luna C18 (2) preparatory LC column with particle size of 10 microns and pore size of 100 Å is used to isolate Compound I from a mixture, wherein the major components are Compound I and Compound II.

### 4. Food Products

The non-artificial blue anthocyanin-containing colorants of the present disclosure, i.e., Compound I, can be used in a colorant composition and can be added to food products, for example, to alter the color characteristics of the food product. The colorant composition can be an edible colorant composition including Compound I with a metal ion at a pH of from about 6 to about 8. For example, and not by way of limitation, the colorant can be used within a coating (i.e., within an edible colorant composition comprising the colorant) for confections to produce a blue-colored coating. Alternatively, or additionally, the non-artificial blue anthocyanin-containing colorant, i.e., Compound I, can be combined with another colorant, e.g., a non-artificial yellow colorant, to produce a non-artificial green colorant.

In certain embodiments, the colorants of the present disclosure can color a food product or a portion thereof.

In certain embodiments, the non-artificial blue anthocyanin-containing colorants can be used in a wide variety of edible products. Non-limiting examples of suitable food products include chocolates, chewing gum compositions, hard and soft confectionery products, dairy products, food products of the beverage category where the product is at about a neutral pH, food products of the frozen food category including frozen dairy products, pharmaceuticals and food categories described herein.

As used herein, “beverage category” can refer to beverages, beverage mixes and concentrates, including but not limited to, alcoholic and non-alcoholic ready to drink and dry powdered beverages, where the beverage is at about a neutral pH. Additional non-limiting examples of beverages can include carbonated and non-carbonated beverages, e.g., sodas, fruit or vegetable juices.

As used herein, “frozen food category” refers to chilled or frozen food products that have a neutral pH. Non-limiting examples of food products of the frozen food category can include ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yogurt, artisanal ice cream, frozen ready meals, frozen pizza, chilled pizza, frozen soup, frozen pasta, frozen processed red meat, frozen processed poultry, frozen processed fish/seafood, frozen vegetables, frozen processed vegetables, frozen meat substitutes, frozen potatoes, frozen bakery products and frozen desserts.

As used herein, “snack food category” refers to any food that can be a light informal meal including, but not limited to sweet and savory snacks and snack bars, where the foods have a neutral pH. Examples of snack foods include, but are not limited to, fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

### 4.1 Confectionary Products

In certain embodiments, an edible colorant composition (e.g., comprising Compound I) of the presently disclosed subject matter can be incorporated into a confectionery product. Non-limiting examples of confectionery products include cakes, cookies, pies, candies, chocolates, chewing gums, gelatins, ice creams, sorbets, puddings, jams, jellies, cereal and other breakfast foods, canned fruits and fruit sauces.

In certain embodiments, an edible colorant composition of the present disclosure can be incorporated into the confections by admixing the edible colorant composition into a confectionery product, e.g., a hard or soft confectionery product. For example, and not by way of limitation, the present disclosure provides methods for enhancing or modulating the blue color of an edible product that comprises (a) providing at least one food product, or a precursor thereof, and (b) combining the food product or precursor thereof with one or more blue edible colorant compositions, disclosed herein, so as to form a modified edible food product. Additionally, the methods can be used for enhancing or modulating the green color of an edible product by (a) providing at least one food product, or a precursor thereof, and (b) combining the food product or precursor thereof with one or more green edible colorant compositions, e.g., an edible colorant composition comprising a blue colorant and a yellow colorant, so as to form a modified edible food product.

In certain embodiments, certain amounts of an edible colorant composition of the present disclosure can be incorporated into a confectionery product. The amount of the edible colorant composition that is used can depend on a number of factors including, but not limited to, the type of bulking agent or carrier employed, method of application, use rate, the type of colorant employed and the intensity of color desired.

In certain embodiments of the present disclosure, the edible colorant composition is admixed with a confection or a component of a confection, where the edible colorant composition is added in amounts to give blue color that can range in intensity from a very pale light cyan blue through to a very dark cyan blue color. The edible colorant composition can pure or nearly pure Compound I, e.g., with metal ion, or the edible colorant composition can contain other ingredients to aid in the incorporation of Compound I into various types of food products. In certain embodiments, the edible colorant composition may contain, for example, from about 0.01% to about 0.1% weight/weight (w/w), or from about 0.07% to about 10% (w/w), or from about 5% to about 35% (w/w) of Compound I. In other embodiments, the edible colorant composition may contain from about 0.005% to about 0.01% (w/w) of Compound I. Depending on the amount of Compound I contained in the colorant composition, more or less colorant composition can be used to achieve the desired color properties in the confection or component of the confection. Such amounts result in an effective use rate, which can be expressed as the percent (w/w) of Compound I added to the confection, confection component, food product or portion thereof. In certain embodiments, the effective use rate of Compound I is an amount of from about 0.0001% to about 10% (w/w), or from about 0.0005% to about 1.0% (w/w), or from about 0.001% to about 0.5% (w/w), or from about 0.005% to about 0.2% (w/w), or from about 0.01% to about 0.1% (w/w), or from about 0.02% to about 0.08% (w/w), and values in between.

In certain embodiments, the edible colorant composition of the present disclosure can be incorporated in a confectionery product of the dragée type, which can include a core and a layer of granulated sugar. Non-limiting examples of the type of cores in a dragée type confectionery product can include a non-artificial center (e.g., almond, hazelnut or groundnut) or a “confectionery” center (e.g., caramel, fondant or chocolate). The cores can then be coated with chocolate, with successive layers of sugars or other substances such as polyols (e.g., erythritol, xylitol, maltitol, or sorbitol), gums and non-artificial polymers, that can further include one or more color compositions of the present disclosure. In certain embodiments, the present disclosure provides for confectionery products that are coated with a blue edible color composition disclosed herein. In other embodiments, the present disclosure provides for confectionery products that are coated with a green edible color composition disclosed herein.

In certain embodiments, the methods for manufacturing compositions of the dragée type can comprise the deposition of a plurality of sublayers, for example between about 5 and about 30, by a succession of phases of application and drying carried out, for example, in a pan. In a hard panning process, multiple applications of a highly concentrated sugar syrup can be used to build up the uncolored portion of a sugar coating on an edible product center. This can be followed by multiple applications of a concentrated sugar syrup containing an edible colorant composition of the present disclosure. In certain embodiments, the hard panning process comprises the repetitive application of thin layers of a coating solution or composition onto an intermixed mass of centers, and the drying of each layer of coating solution or composition during which the sugar in the coating crystallizes between the applications of layers. Additional non-limiting examples of methods for producing hard panned confectionaries are provided in International Patent Publication Nos. WO 2014/150438 and WO 2014/152417, the disclosures of which are incorporated herein by reference.

In certain embodiments, where a coating is to be colored, an edible colorant composition of the present disclosure can be added to the coating solution in the later stages of the coating process. For example, and not by way of limitation, the edible colorant composition comprises a monoacylated anthocyanin, e.g., of Compound I, a metal ion, or salt form thereof, and a pH adjusting ingredient having a pH of about 6 to about 8. For a hard panned confectionery, following the application of a number of layers of the uncolored sugar syrup to build up the sugar coating, a number of applications of a sugar syrup comprising an edible colorant composition, disclosed herein, are applied to provide the color coat. In certain embodiments, the color coat can require 30 or more applications of a colored coating comprising the edible colorant composition solution to achieve the desired color.

In certain embodiments, when an edible colorant composition is included in one or more sugar syrups used for hard panned coating, the hard panned coating has a visible color provided by the edible colorant composition. A hard panned confection comprising an edible product center coated with this same hard panned coating also has a visible color provided by the edible colorant composition. In certain embodiments, the hard panned coating has a blue color, and a hard panned confection coated with this same hard panned coating has a blue color. The blue color can be provided, at least in part, by a blue edible colorant composition, disclosed herein, incorporated in the coating.

In certain embodiments, the hard panned coating has a green color, and a hard panned confection coated with this same hard panned coating has a green color. In certain embodiments, the green color can be provided by an edible colorant composition comprising the combination of a blue colorant (e.g., Compound I with a metal ion), disclosed herein, and a non-artificial yellow colorant incorporated in the coating. Examples of non-artificial yellow colorants can include, but are not limited to, curcuminoids (e.g., from turmeric), carotenoids (e.g., from saffron, gac fruit, and gardenia), annatto (e.g., from achiote) and combinations thereof. In certain embodiments, the non-artificial yellow colorant is derived from turmeric. In other embodiments, the non-artificial yellow colorant is gardenia yellow.

In certain embodiments, the present disclosure provides edible products in the form of a hard coated confectionery product comprising a center core and at least one coating layer that comprises the edible colorant composition disclosed herein and crystallized sugar. For example, and not by way of limitation, the present disclosure provides a hard panned confection that comprises (a) an edible core and (b) a hard panned coating that comprises a plurality of coating layers, wherein at least one of the coating layers comprises an edible colorant composition of the present disclosure. For example, and not by way of limitation, the edible core can comprise chocolate.

### 5. Methods of Preparation of the Colorant Compositions

Edible colorant compositions containing Compound I can be either liquid or solid preparations. In certain embodiments, Compound I is dissolved in water, and the pH is adjusted to a pH value in the range of about 6 to about 8, with about pH 7 being optimal. In certain embodiments, from about 0.3 to about 1.0 mol. equiv. (e.g., about ⅓ mol. equiv. or about 1 mol. equiv.) of an edible aluminum or iron salt is added to the solution. If the final format is to be a dispersible solid, then a suitable dissolved solid such as a maltodextrin is dissolved in the colorant solution. The total dissolved solids should be about 10-15% of the colorant solution, and this material can them be spray dried using an apparatus known in the art. The content of Compound I can be from about 1% to about 10% (e.g., about 2% to about 9%, about 3% to about 7%, about 4% to about 6%, about 2% to about 7%, about 2% to about 4%, about 5% to about 7%, or about 5% to about 9%) of the final dried colorant composition. When a liquid preparation is preferred, it may be preferable to maintain the solution at a low pH, about 2-3 to improve the long term stability of Compound I. From about 0.3 to about 1.0 mol. equiv. (e.g., about ⅓ mol. equiv. or about 1 mol. equiv.) of an edible aluminum or iron salt is added to the solution.

In certain embodiment, suitable excipients, known in the art, can be added to help maintain microbiological stability of the color composition. Refrigerated or frozen storage of a liquid color composition is preferred for maximum stability.

### EXAMPLES

The presently disclosed subject matter will be better understood by reference to the following Examples, which are provided as exemplary of the disclosure and should not be construed as limiting the scope of the disclosure in any way.

### Example 1: Design of Esterases for Selectively Deacylation of Anthocyanins

Compound I is a compound that can be used in food products as a non-artificial blue colorant, but challenges have arisen in isolating sufficient quantities of the pure product of Compound I from a mixture of similar compounds. It is contemplated herein that the amount of Compound I in a composition can be enriched by modifying an enzyme to specifically catalyze the synthesis of Compound I from a mixture of several anthocyanin compounds.

### Database Mining and Sequence Comparison

In the first round of database mining, we searched BRENDA for representative enzymes from a variety of enzyme classifications, favoring genes with crystal structures. In round 2 of mining, we looked specifically at genes active on substrates similar to anthocyanins, as well as enzymes that perform similar chemistry to the active gene found in round 1, #37 of Table 9. Genome mining in round 3 was completed using phmmer and jackhammer searches on HMMER, an online application that uses Hidden Markov Model to identify sequences similar to a submitted target sequence (Potter et al., “HMMER web server: 2018,” Nucleic Acids Research. 46(W1):W200-W204, 2018). Sequences were selected from the PDB and Ensembl Genomes Bacteria databases. Sequences were aligned and percentage identities calculated using the MUSCLE Alignment tool on Geneious 10.1.3.

A table of the identified enzymes is provided below in Table 9. A table of the sequences of each of the enzymes tested is provided below in Table 10.

TABLE 9
Enzymes tested in study

| ID # | EC # | UniProt ID | PDB | Enzyme Classification | Domain |
|---|---|---|---|---|---|
| 1 | 3.1.1.1 | Q1QYJ5 | | Carboxylesterase | Bacteria |
| 2 | 3.1.1.1 | A0A1J8PUW1 | | Carboxylesterase | Bacteria |
| 3 | 3.1.01.- | Q5NI32 | 4F21 | Carboxylesterase/phospholipase | Bacteria |
| 4 | 3.1.1.57 | O87170 | 4DI9 | 2-pyrone-4,6-dicarbaxylate hydrolase | Bacteria |
| 5 | 3.1.1.45 | P0A114 | 1ZI8 | Dienelactonase mutant | Bacteria |
| 6 | 3.1.1.1 | Q06174 | 1TQH | Carboxylesterase | Bacteria |
| 7 | 3.1.3.1 | B8Y562 | 4RGY | Alkaline esterase | Bacteria |
| 8 | 3.1.1.91 | F8QQ74 | | 2-oxo-3-(5-oxofuran-2-ylidene) | Bacteria |
| | | | | propanoate lactonase | |
| 9 | 3.5.2.6 | F8CAF0 | | Beta-lactamase | Bacteria |
| 10 | 3.1.1.27 | Q988B9 | 3AJ3 | 4-pyridoxolactonase | Bacteria |
| 11 | 3.1.1.92 | A6XIG7 | | 4-sulfomuconolactone hydrolase | Bacteria |
| 12 | 3.5.2.6 | B2BSN6 | | Beta-lactamase | Bacteria |
| 13 | 3.5.2.6 | P00811 | 2FFY | Beta-lactamase | Bacteria |
| 14 | 3.1.1.65 | A3LZU8 | | L-rhamnono-gamma-lactonase | Eukaryote |
| 15 | 3.1.8.1 | P15034 | 2V3Z | Xaa-Pro aminopeptidase | Bacteria |
| 16 | 3.1.1.68 | R0CVD2 | | Xylono-1,4-lactonase | Bacteria |
| 17 | 3.1.1.1 | G4RFI7 | 5DWD | Esterase | Bacteria |
| 18 | 3.1.1.83 | Q9EX73 | | Monoterpene epsilon-lactone hydrolase | Bacteria |
| 19 | 3.1.1.15 | Q1JUP5 | | L-arabinolactonase | Bacteria |
| 20 | 3.2.1.8 | P51584 | 1GKK | Feruloyl esterase | Bacteria |
| 21 | 3.1.1.17 | B0RN69 | 3DR2 | Exported gluconolactonase | Bacteria |
| 22 | 3.1.1.73 | O42807 | 1USW | Ferulic acid esterase | Bacteria |
| 23 | 3.1.1.25 | B2LYJ5 | | Levo-lactonase | Eukaryote |
| 24 | 3.1.1.17 | A9CPS8 | | Lactonase | Eukaryote |
| 25 | 3.5.2.6 | Q8GCU7 | | Metallo-beta-lactamase VIM-6 | Bacteria |
| 26 | 3.5.2.6 | Q8KRJ3 | 2WHG | Beta-lactamase VIM-4 | Bacteria |
| 27 | 3.1.1.84 | Q9L9D7 | 1JU3 | Cocaine esterase | Bacteria |
| 28 | 3.1.1.31 | Q9GRG6 | 3EB9 | 6-phosphogluconolactonase | Eukaryote |
| 29 | 3.1.1.24 | Q13KT2 | 2XUA | 3-oxoadipate enol-lactonase | Bacteria |
| 30 | 3.1.1.1 | A0A0M3KKY6 | 4UHC | Carboxylesterase | Bacteria |
| 31 | 3.1.1.95 | Q54528 | 1QOR | Aclacinomycin methylesterase | Bacteria |
| 32 | 3.1.1.81 | P0CJ63 | 3DHA | N-acyl homoserine lactonase AiiA | Bacteria |
| 33 | 3.1.1.11 | P0C1A9 | 2NSP | Pectinesterase A | Bacteria |
| 34 | 3.1.1.1 | O28558 | 1JJI | Carboxylesterase | Archaea |
| 35 | 3.1.1.4 | A0A243LS46 | | Esterase | Bacteria |
| 36 | 3.1.1.1 | Q7SIG1 | 1EVQ | Carboxylesterase | Bacteria |
| 37 | 3.1.1.2 | B5BLW5 | 5L2P | Arylesterase | Archaea |
| 38 | 3.1.1.1 | F0NDQ1 | 5LK6 | Lipase carboxylesterase | Bacteria |
| 39 | 3.1.1.1 | Q976W8 | 3AIK | Carboxylesterase | Bacteria |
| 40 | 3.1.1.1 | A0A139SSC5 | | Carboxylesterase | Bacteria |
| 41 | 3.1.1.1 | A0A2U2AP80 | | Carboxylesterase | Bacteria |
| 42 | 3.1.1.1 | L8MEL5 | | Carboxylesterase | Bacteria |
| 43 | 3.1.1.1 | A0A127MYW2 | | Carboxylesterase | Bacteria |
| 44 | 3.1.1.1 | Q9HXE7 | 3CN9 | Carboxylesterase | Bacteria |
| 45 | 3.1.1.1 | A0A078LXQ1 | | Alpha/beta-hydrolase | Bacteria |
| 46 | 3.1.1.1 | Q53547 | 1AUR | Carboxylesterase | Bacteria |

TABLE 10
Amino acid sequences of enzymes tested in Example 1

| SEQ | | |
|---|---|---|
| ID NO: | ID# | Sequence |
| 44 | 1 | MADSSPLIIEPRQARAADATVILLHGLGADGHDFEPLVPALPLAKDLAVRFVLP |
| | | HAPRMPVTVNGGMEMPAWYDILDMNLGRRIDEAQLKASADMVHGLIDAEIA |
| | | RGIDSRRIIVAGFSQGGAVAYHAALTYPKPLGGLLALSTYFATATSIEPSEANR |
| | | ALPIEVHHGSFDPVVPEALGHEGAERAEALGYAVTYRTYPMQHALCPEQIEDI |
| | | GQWLNARLGAKEAGGSLEHHHHHH |
| 45 | 2 | MTAPGELIIEPKDGQPADACVFIIHGLGADGHDFEPLVPALALPKDSRVRFIMP |
| | | HAPRLPVTINGGMVMPAWYDILAMDLGRRVDERQLKQSAERIQALIQEQIDQ |
| | | GIDSQRIIVAGFSQGGAVAYHAALTFPAPLGGLLAMSTYFATADNIDLAEANR |
| | | QIPIEVQHGNFDPIVPESLGRSGADRLKEMGYAVNYRQYPMAHALCPQQVNDI |
| | | GKWLSARLNGGSLEHHHHHH |
| 46 | 3 | MNYELMEPAKQARFCVIWLHGLGADGHDFVDIVNYFDVSLDEIRFIFPHADIIP |
| | | VTINMGMQMRAWYDIKSLDANSLNRVVDVEGINSSIAKVNKLIDSQVNQGIAS |
| | | ENIILAGFSQGGIIATYTAITSQRKLGGIMALSTYLPAWDNFKGKITSINKGLPIL |
| | | VCHGTDDQVLPEVLGHDLSDKLKVSGFANEYKHYVGMQHSVCMEEIKDISNF |
| | | IAKTFKIGGSLEHHHHHH |
| 47 | 4 | MTNDERILSWNETPSKPRYTPPPGAIDAHCHVFGPMAQFPFSPKAKYLPRDAG |
| | | PDMLFALRDHLGFARNVIVQASCHGTDNAATLDAIARAQGKARGIAVVDPAI |
| | | DEAELAALHEGGMRGIRFNFLKRLVDDAPKDKFLEVAGRLPAGWHVVIYFEA |
| | | DILEELRPFMDAIPVPIVIDHMGRPDVRQGPDGADMKAFRRLLDSREDIWFKA |
| | | TCPDRLDPAGPPWDDFARSVAPLVADYADRVIWGTDWPHPNMQDAIPDDGL |
| | | VVDMIPRIAPTPELQHKMLVTNPMRLYWSEEMGGSLEHHHHHH |
| 48 | 5 | MLTEGISIQSYDGHTFGALVGSPAKAPAPVIVIAQEIFGVNAFMRETVSWLVDQ |
| | | GYAAVCPDLYARQAPGTALDPQDERQREQAYKLWQAFDMEAGVGDLEAAIR |
| | | YARHQPYSNGKVGLVGYCLGGALAFLVAAKGYVDRAVGYYGVGLEKQLKK |
| | | VPEVKHPALFHMGGQDHFVPAPSRQLITEGFGANPLLQVHWYEEAGHSFART |
| | | SSSGYVASAAALANERRLDFLAPLQSKKPGGSLEHHHHHH |
| 49 | 6 | MKIVPPKPFFFEAGERAVLLLHGFTGNSADVRMLGRFLESKGYTCHAPIYKGH |
| | | GVPPEELVHTGPDDWWQDVMNGYEFLKNKGYEKIAVAGLSLGGVFSLKLGY |
| | | TVPIEGIVTMCAPMYIKSEETMYEGVLEYAREYKKREGKSEEQIEQEMEKFKQ |
| | | TPMKTLKALQELIADVRDHLDLIYAPTFVVQARHDEMINPD SANIIYNEIESPV |
| | | KQIKWYEQSGHVITLDQEKDQLHEDIYAFLESLDWGGSLEHHHHHH |
| 50 | 7 | MALFQCDFFSDVLGLSTSMTVILPQETTGQIGMAGGSERREHPTLFLLHGLSDD |
| | | HTIWLRRTSIERYVAEMGLAVVMPAVHRSFYTDMAHGLQYWTFISEELPALA |
| | | RSFFPLATAREDTFVAGLSMGGYGALKLGMRHPERFAAAASLSGALDITFDPA |
| | | EHIAMEDDVWVAEQRNIFGDLAALPGSDHDLFALAERMAQSDGPVPKLYQCC |
| | | GTEDFLYEDNVRFRDHVRGLGLDFMYEESPGEHEWGYWDAQIQRVLAWLPL |
| | | RPPGTAPAGGSLEHHHHHH |
| 51 | 8 | MATETIAMDWVDIGTNGESRLAYLARPVVTGRLPAVIVMPAIHGINTYIKDVA |
| | | IDLAKAGFVALLIDIHSPEQEPDLSNAEKIQIAVETLDDRKVLKDVDAAVRYLE |
| | | QHAAVRADRLGILGFCVGGTYALLAARTPAIRVSVGFYGLLEYQSRTDNKPVS |
| | | PLDSVAQFTAPILFHVGDKDPWIDSKMLAEFTKRMQQHQKSYELCIYRGAGH |
| | | AFHEHFRDAYRPIAAQSAWNNTLIYLRWHLCGKRTVGGSLEHHHHHH |
| 52 | 9 | MNGLRWRLTGVVMAWVLVAPLAEAANVKQEVDRYISGFHQKGLFNGTVLV |
| | | ANERGILLKKGYGAANLEWKVPNAPDTKFRIGSITKSFTATVILQLAAEGKLQL |
| | | DDPITKHLPDYRKDTGDRVTITHLLNHTSGIPSYTSKPAIMKDADGFESVAAFV |
| | | KKACSDDLEFEPGTKYAYNNSGYFLLGAIIEKLTGQTYAEAVQARILGPLGMK |
| | | DTGYDVSATVLPKRASGYAQAPGGIVNAAWLDMNLPYAAGSLYSTVEDLYR |
| | | WERAFHGDTLLPAALKQKMLTPGLAHYGFGWVMSDMTLHDGKTKLPGIFHT |
| | | GGINGFSSILVRVPERKEAVILLDNMTHGGLQELAGGVLSILHGLTPRPARMPI |
| | | GNVMMESLGKGSVAQAIATYRTLKKTKQAEYDFSERHLNTVGYHLLRSGRA |
| | | ADAIEVFKLNVEMFPEAANCHDSLGEAYAAHGDKARAITSYRKALELAPKNE |
| | | HAVKMLEQLEEPAAKRGGSLEHHHHHH |
| 53 | 10 | MSDTKVYLLDGGSLVLDGYHVFWNRGPGGEVRFPVYSILIEHAEGRFLIDTGY |
| | | DYDHVMKVLPFEKPIQEKHQTIPGALGLLGLEPRDIDVVVNSHFHFDHCGGNK |
| | | YFPHAKKICHRSEVPQACNPQPFEHLGYSDLSFSAEAAEARGATAQLLEGTTR |
| | | ANSTFEGIDGDVDLARGVKLISTPGHSIGHYSLLVEFPRRKPILFTIDAAYTQKS |
| | | LETLCQAAFHIDPVAGVNSMRKVKKLAEDHGAELMYSHDMDNFKTYRTGTQ |
| | | FYGGGSLEHHHHHH |
| 54 | 11 | MLPADQAGIPPCQGPRARSAPISFAIPKGAWDTHLHVFGPTAVFPYAEKRPYTP |
| | | PDSPLEDYLALMERLGIERGVCVHPNVHGIDNSVTIDAVERSDRRLLGIIKPHR |
| | | VMTFTELRDLKTRGVRGVRFAFNPQHGSGALDTELFERMHGWCRELDWCIN |
| | | MHFAPDALEGLCDLIAGAETPIIIDHFGRVETAAGVNQLPFKILRDLATLDHVW |
| | | IKLTGADRISHSGVPYDDVVPFAHALSEIAPDRLLWGSDWPHSGYFDPKRMPD |
| | | DGDLLNLVARFAPDVALRHKILVDNPARLFGVIGGSLEHHHHHH |
| 55 | 12 | MKQRIALSLLALGPLLLVPRVYAAADEPMANIVEKAVQPLLEEYRIPGMAVAV |
| | | LKEGKPHYFNYGVANRESGRRISERTLFEIGSVSKTFTATLGTYAVVKGGFRLD |
| | | DKVSQHAPWLQNSAFDRVTMAQLATYSAGGLPLQFPDAVDSNERMRQYYRQ |
| | | WSPLYAAGTHREYSNPSIGLFGHLAASTLGQPFRQLMSQTLLPKLDLQHTYLE |
| | | VPDAAMVDYAYGYSKEDKPVRVNPGVLADEAYGIKTSAADLIKFVGANMTG |
| | | SGDKAVQQALAMTRTGFYSVGEMTQGLGWESYAYPVTEQALLAGNSPAVSF |
| | | KANPVKPFVAPRVMGNERLYNKTGSTNGFGAYVVFVPARGVGIVMLANRNY |
| | | PIEARVKAAYAIMRHLAPGGSLEHHHHHH |
| 56 | 13 | MFKTTLCALLITASCSTFAAPQQINDIVHRTITPLIEQQKIPGMAVAVIYQGKPY |
| | | YFTWGYADIAKKQPVTQQTLFELGSVSKTFTGVLGGDAIARGEIKLSDPTTKY |
| | | WPELTAKQWNGITLLHLATYTAGGLPLQVPDEVKSSSDLLRFYQNWQPAWAP |
| | | GTQRLYANSSIGLFGALAVKPSGLSFEQAMQTRVFQPLKLNHTWINVPPAEEK |
| | | NYAWGYREGKAVHVSPGALDAEAYGVKSTIEDMARWVQSNLKPLDINEKTL |
| | | QQGIQLAQSRYWQTGDMYQGLGWEMLDWPVNPDSIINGSDNKIALAARPVK |
| | | AITPPTPAVRASWVHKTGATGGFGSYVAFIPEKELGIVMLANKNYPNPARVDA |
| | | AWQILNALQGGSLEHHHHHH |
| 57 | 14 | MSKYKILDSHIHLYSLANIPLLHWDEGNPLHGNRRLDEYIENSQSTQFDVEGV |
| | | VWIECDAKIDLTQGLKGLENPIEEYLYICRNINGKLLPEEGVSTPFKRRLIKAMI |
| | | PFAPMPLGSAGVEEYVKALKTRNSSEFHLVKGFRYLIQDKPPLTISDPHFVSSF |
| | | QWLDSNGYVFDLGIDMRSGGLWQFKETLEVFKKVPNLKYIINHLTKPCLDFDP |
| | | ETIDSNPDFLSWKRLVTEMYITTPNSYMKLSGGFSEVEQDVALDVTSTSRHVY |
| | | PWFKVVYELWGPERTIFASNWPVCAIPAGQNLTEKWFQVCETLFDSIGMDEDT |
| | | RRKIYYSNAFKAYNIGGSLEHHHHHH |
| 58 | 15 | MSEISRQEFQRRRQALVEQMQPGSAALIFAAPEVTRSADSEYPYRQNSDFWYF |
| | | TGFNEPEAVLVLIKSDDTHNHSVLFNRVRDLTAEIWFGRRLGQDAAPEKLGVD |
| | | RALAFSEINQQLYQLLNGLDVVYHAQGEYAYADVIVNSALEKLRKGSRQNLT |
| | | APATMIDWRPVVHEMRLFKSPEEIAVLRRAGEITAMAHTRAMEKCRPGMFEY |
| | | HLEGEIHHEFNRHGARYPSYNTIVGSGENGCILHYTENECEMRDGDLVLIDAG |
| | | CEYKGYAGDITRTFPVNGKFTQAQREIYDIVLESLETSLRLYRPGTSILEVTGEV |
| | | VRIMVSGLVKLGILKGDVDELIAQNAHRPFFMHGLSHWLGLDVHDVGVYGQ |
| | | DRSRILEPGMVLTVEPGLYIAPDAEVPEQYRGIGIRIEDDIVITETGNENLTASVV |
| | | KKPEEIEALMVAARKQGGSLEHHHHHH |
| 59 | 16 | MNAVTCVWDLKATLGEGPIWYDDSLWFVDIKSHKIHNYNPATDERFSFDAPE |
| | | PVTFIAPLAPNARAGFVVGLKSGLHRFHPVMGGFKPLIQVESAELNNRPNDAT |
| | | VDHGGRLWFGTMHDDEEAKSGSLYRMDSTGVARMDKDICITNGPCVSPDGK |
| | | TLYHTDTLEKIIWAYDLAEDGTLSNKRGFVNFQGENAVYPDGSVVDSEGYLW |
| | | TALWGGFGVVRISPAGELVARIELPAPNVTKPCFGGPDLKTLYFTTARKGLSDE |
| | | TLAQYPLSGGLFGVRVDVAGQPQHEVRLVGGSLEHHHHHH |
| 60 | 17 | MTEPVKLSGPMLPAVSGAAKSLVVLLHGYGSDGRDLIALGQFWRDSFPDTMF |
| | | VAPNAPHVCGGNPFGYEWFPLDLERDRTLARLAGAETAHPVLDAFLADLWA |
| | | QTGLGPADTILVGFSQGAMMALYTGLRLPEPLKAIIAFSGLIVAPEKLEAEIASK |
| | | PPVLLIHGDLDDVVPVIGSETALPKLIDLGIDARLHISQGSGHTIAQDGLDTATA |
| | | FLREILGGSLEHHHHHH |
| 61 | 18 | MSATDTARAKELLASLVSMPDATIDDFRALYEQVCATFELPDDAQVEPVDAN |
| | | GADALWVSAPGVSADTVAVVVHGGGFTMGSAHGYRELGYRLSKSGNLRALV |
| | | VDYRLAPESPFPAPVDDVVAAYRYARSLDGVENVFLVGDSAGGGIAMSALITL |
| | | RDAGEQLPDAAVVLSPLVDLAGESPSLVDRAHLDPLPAAVLVNGMGGLYLNG |
| | | LDVRHPVASPMHGDLTGLPATLVLVGTDEGLHDDSTRLVDKLKAADVEVQLE |
| | | IGEGLPHIWPIFSFHPDAVAATDRIGEFLRSHVAAPRGGSLEHHHHHH |
| 62 | 19 | MQQIHPAGQATLLADTRNTLGEGATWCDRTRALYWVDIEGAQLWRCRADGS |
| | | DLTPWPMPERLACFALTDDPDVLLVGLATHLAFFDLRSGAFTRIVEVEPELPTR |
| | | LNDGRCDGSGAFVFGMKDEGAEPPRAVGGFYRLNADLTLERLALPPAAIANSI |
| | | GFSPDGSKMYFCDSLVREIFVCDYRPGGEVANVRPFARLTDPDGDPDGSIVDR |
| | | DGGLWNAQWGGRRVVRYGPDGVETDRVAVPTAQPSCTALDGEGRLYVTSA |
| | | RVGLSDDALADDPHAGGVFVAQTRHAGMATARFAGTPRGGGSLEHHHHHH |
| 63 | 20 | MKNKRVLAKITALVVLLGVFFVLPSNISQLYADYEVVHDTFEVNFDGWCNLG |
| | | VDTYLTAVENEGNNGTRGMMVINRSSASDGAYSEKGFYLDGGVEYKYSVFV |
| | | KHNGTGTETFKLSVSYLDSETEEENKEVIATKDVVAGEWTEISAKYKAPKTAV |
| | | NITLSITTDSTVDFIFDDVTITRKGMAEANTVYAANAVLKDMYANYFRVGSVL |
| | | NSGTVNNSSIKALILREFNSITCENEMKPDATLVQSGSTNTNIRVSLNRAASILN |
| | | FCAQNNIAVRGHTLVWHSQTPQWFFKDNFQDNGNWVSQSVMDQRLESYIKN |
| | | MFAEIQRQYPSLNLYAYDVVNEAVSDDANRTRYYGGAREPGYGNGRSPWVQ |
| | | IYGDNKFIEKAFTYARKYAPANCKLYYNDYNEYWDHKRDCIASICANLYNKG |
| | | LLDGVGMQSHINADMNGFSGIQNYKAALQKYINIGCDVQITELDISTENGKFSL |
| | | QQQADKYKAVFQAAVDINRTSSKGKVTAVCVWGPNDANTWLGSQNAPLLFN |
| | | ANNQPKPAYNAVASIIPQSEWGDGNNPAGGGGGGKPEEPDANGYYYHDTFEG |
| | | SVGQWTARGPAEVLLSGRTAYKGSESLLVRNRTAAWNGAQRALNPRTFVPG |
| | | NTYCFSVVASFIEGASSTTFCMKLQYVDGSGTQRYDTIDMKTVGPNQWVHLY |
| | | NPQYRIPSDATDMYVYVETADDTINFYIDEAIGAVAGTVIEGPAPQPTQPPVLL |
| | | GDVNGDGTINSTDLTMLKRSVLRAITLTDDAKARADVDKNGSINSTDVLLLSR |
| | | YLLRVIDKFPVAENPSSSFKYESAVQYRPAPDSYLNPCPQAGRIVKETYTGING |
| | | TKSLNVYLPYGYDPNKKYNIFYLMHGGGENENTIFSNDVKLQNILDHAIMNGE |
| | | LEPLIVVTPTFNGGNCTAQNFYQEFRQNVIPFVESKYSTYAESTTPQGIAASRM |
| | | HRGFGGFSMGGLTTWYVMVNCLDYVAYFMPLSGDYWYGNSPQDKANSIAE |
| | | AINRSGLSKREYFVFAATGSDHIAYANMNPQIEAMKALPHFDYTSDFSKGNFY |
| | | FLVAPGATHWWGYVRHYIYDALPYFFHEGGSLEHHHHHH |
| 64 | 21 | MDSHCRVRPAGPAVPADCDPPRITHAALAARLGDARLLTLYDQATWSEGPA |
| | | WWEAQRTLVWSDLVGRRVLGWREDGTVDVLLDATAFTNGNAVDAQQRLV |
| | | HCEHGRRAITRSDADGQAHLLVGRYAGKRLNSPNDLIVARDGAIWFTDPPFGL |
| | | RKPSQGCPADPELAHHSVYRLPPDGSPLQRMADLDHPNGLAFSPDEQTLYVSQ |
| | | TPEQGHGSVEITAFAWRDGALHDRRHFASVPDGLPDGFCVDRGGWLWSSSGT |
| | | GVCVFDSDGQLLGHIPTPGTASNCTFDQAQQRLFITGGPCLWMLPLPGGSLEH |
| | | HHHHH |
| 65 | 22 | MKQFSAKYALILLATAGQALAASTQGISEDLYNRLVEMATISQAAYADLCNIP |
| | | STIIKGEKIYNAQTDINGWILRDDTSKEIITVFRGTGSDTNLQLDTNYTLTPFDTL |
| | | PQCNDCEVHGGYYIGWISVQDQVESLVKQQASQYPDYALTVTGHSLGASMA |
| | | ALTAAQLSATYDNVRLYTFGEPRSGNQAFASYMNDAFQVSSPETTQYFRVTH |
| | | SNDGIPNLPPADEGYAHGGVEYWSVDPYSAQNTFVCTGDEVQCCEAQGGQG |
| | | VNDAHTTYFGMTSGACTWGGSLEHHHHHH |
| 66 | 23 | MPSSISVLAAGILVPVLGAVAAKLPPTAQIIDQKSFNVLKDVPPPAVANDSLVF |
| | | TWPGVTEESLVEKPFHVYDEEFYDVIGKDPSLTLIATSDTDPIFHEAVVWYPPT |
| | | EEVFFVQNAGAPAAGTGLNKSSIIQKISLKEADEVRKGKKDEVKVTVVDSNPQ |
| | | VINPNGGTYYKGNIIFAGEGQGDDVPSALYLMNPLPPYNTTTLLNNYFGRQFN |
| | | SLNDVGINPRNGDLYFTDTLYGYLQDFRPVPGLRNQVYRYNFDTGAVTVVAD |
| | | DFTLPNGIGFGPDGKKVYVTDTGIALGFYGRNLSSPASVYSFDVNQDGTLQNR |
| | | KTFAYVASFIPDGVHTDSKGRVYAGCGDGVHVWNPSGKLIGKIYTGTVAANF |
| | | QFAGKGRMIITGQTKLFYVTLGASGPKLYDGGSLEHHHHHH |
| 67 | 24 | MRTLATVASQTDAWTGEGPVWCAARRCLYYVDLGDTRPGKLHVYHPERCVE |
| | | EIHDLPAMTKDFTQVTAVTVVQNEPHRLAVATEAGVFLYDCQSGDLRRLTGE |
| | | LQPELPKGSYRSNDGKCDPRGRFLIGTMLFSADAPSGGLFSVAGSTIQQLLTGV |
| | | TIGNGLAWSANGRTMYFIDSPLKRIDAFEYHLDAGTLGARRTAFDFADYFAQQ |
| | | AGWEEAAPDGMTIDAEGLLWVAIYGGGAALRVDPAKEEVVCRVDCPAKYTT |
| | | SVALGGPARDTLYITSFRRGDAGPDAGAVFQCRAPAPGPPPAEFRLGGSLEHH |
| | | HHHH |
| 68 | 25 | MFKLLSKLLVYLTASIMAIASPLAFSVDSSGEYPTVSEIPVGEVRLYQIADGVW |
| | | SHIATRSFDGAVYPSNGLIVRDGDELLLIDTAWGAKNTAALLAEIEKQIGLPVT |
| | | RAVSTHFHDDRVGGVDVLRAAGVATYASPSTRRLAEVEGSEIPTHSLEGLSSS |
| | | GDAVRFGPVELFYPGAAHSTDNLVVYVPSASVLYGGCAIYELSRTSAGNVAD |
| | | ADLAEWPTSIERIQQHYPEAQFVIPGHGLPGGLDLLKHTTNVVKAHTNRSVVE |
| | | GGSLEHHHHHH |
| 69 | 26 | MLKVISSLLVYMTASVMAVASPLAHSGEPSGEYPTVNEIPVGEVRLYQIADGV |
| | | WSHIATQSFDGAVYPSNGLIVRDGDELLLIDTAWGAKNTAALLAEIEKQIGLPV |
| | | TRAVSTHFHDDRVGGVDVLRAAGVATYASPSTRRLAEAEGNEIPTHSLEGLSS |
| | | SGDAVRFGPVELFYPGAAHSTDNLVVYVPSANVLYGGCAVHELSRTSAGNVA |
| | | DADLAEWPTSVERIQKHYPEAEVVIPGHGLPGGLDLLQHTANVVKAHKNRSV |
| | | AEGGSLEHHHHHH |
| 70 | 27 | MVDGNYSVASNVMVPMRDGVRLAVDLYRPDADGPVPVLLVRNPYDKFDVF |
| | | AWSTQSTNWLEFVRDGYAVVIQDTRGLFASEGEFVPHVDDEADAEDTLSWIL |
| | | EQAWCDGNVGMFGVSYLGVTQWQAAVSGVGGLKAIAPSMASADLYRAPWY |
| | | GPGGALSVEALLGWSALIGTGLITSRSDARPEDAADFVQLAAILNDVAGAASV |
| | | TPLAEQPLLGRLIPWVIDQVVDHPDNDESWQSISLFERLGGLATPALITAGWYD |
| | | GFVGESLRTFVAVKDNADARLVVGPWSHSNLTGRNADRKFGIAATYPIQEAT |
| | | TMHKAFFDRHLRGETDALAGVPKVRLFVMGIDEWRDETDWPLPDTAYTPFYL |
| | | GGSGAANTSTGGGTLSTSISGTESADTYLYDPADPVPSLGGTLLFHNGDNGPA |
| | | DQRPIHDRDDVLCYSTEVLTDPVEVTGTVSARLFVSSSAVDTDFTAKLVDVFP |
| | | DGRAIALCDGIVRMRYRETLVNPTLIEAGEIYEVAIDMLATSNVFLPGHRIMVQ |
| | | VSSSNFPKYDRNSNTGGVIAREQLEEMCTAVNRIHRGPEHPSHIVLPIIKRGGSL |
| | | EHHHHHH |
| 71 | 28 | MSFKPTISVHATPQELSAAGCRKIVEHEASGSQQWPLSIALAGGSTPKMTYARL |
| | | HDEHLNLLREKRALRFFMGDERMVPADSTDSNYNMAREVLLHDIPDDLVFPF |
| | | DTSAVTPSAEATSADAMRVAEAYGKQLASLLPLKSVGEAGPKVPVFDVVLLG |
| | | LGSDGHTASIFPGSQAEKETDGKVVVSVGFPSETMKPKVWRVTLSPATIMQAR |
| | | NVIVLATGAEKKWVVDGILADTAHKAPVARFLRGCEGNVSFLLDKEIAENLA |
| | | KFGGSLEHHHHHH |
| 72 | 29 | MPYAAVNGTELHYRIDGERHGNAPWIVLSNSLGTDLSMWAPQVAALSKHFR |
| | | VLRYDTRGHGHSEAPKGPYTIEQLTGDVLGLMDTLKIARANFCGLSMGGLTG |
| | | VALAARHADRIERVALCNTAARIGSPEVWVPRAVKARTEGMHALADAVLPR |
| | | WFTADYMEREPVVLAMIRDVFVHTDKEGYASNCEAIDAADLRPEAPGIKVPA |
| | | LVISGTHDLAATPAQGRELAQAIAGARYVELDASHISNIERADAFTKTVVDFLT |
| | | EQKGGSLEHHHHHH |
| 73 | 30 | MAQRVKITTTATPGEIELAFEDTGTGLPVLLVHGFPLDRTMWKAQREELCDEF |
| | | RVIVPDLRGFGESQVIPGVATMEAMADDLAGLCNHLGLTGKIVLGGLSMGGY |
| | | VAFAFARKYRDRLAGLILCDTRARPDSPEAKENRRRVAERVRREGPGFIAEEM |
| | | IPRLCCESTFRNHPEVIEKIRQMILSAPPEGVAAAALGMAERPDSTDLLPALSCP |
| | | TLVLVGQFDAISPPEEMEAMARTIPQSQFVVIPDAGHLPPMEQPERVTQAIREW |
| | | LRKVHTEAGGSLEHHHHHH |
| 74 | 31 | MSERIVPSGDVELWSDDFGDPADPALLLVMGGNLSALGWPDEFARRLADGGL |
| | | HVIRYDHRDTGRSTTRDFAAHPYGFGELAADAVAVLDGWGVDRAHVVGLSM |
| | | GATITQVIALDHHDRLSSLTMLLGGGLDIDFDANIERVMRGEPTLDGLPGPQQP |
| | | FLDALALMNQPAEGRAAEVAKRVSKWRILSGTGVPFDDAEYARWEERAIDHA |
| | | GGVLAEPYAHYSLTLPPPSRAAELREVTVPTLVIQAEHDPIAPAPHGKHLAGLI |
| | | PTARLAEIPGMGHALPSSVHGPLAEVILAHTRSAAGGSLEHHHHHH |
| 75 | 32 | MTVKKLYFIPAGRCMLDHSSVNSALTPGKLLNLPVWCYLLETEEGPILVDTGM |
| | | PESAVNNEGLFNGTFVEGQILPKMTEEDRIVNILKRVGYEPDDLLYIISSHLHFD |
| | | HAGGNGAFTNTPIIVQRTEYEAALHREEYMKECILPHLNYKIIEGDYEVVPGVQ |
| | | LLYTPGHSPGHQSLFIETEQSGSVLLTIDASYTKENFEDEVPFAGFDPELALSSIK |
| | | RLKEVVKKEKPIIFFGHDIEQEKSCRVFPEYIGGSLEHHHHHH |
| 76 | 33 | MLKTISGTLALSLIIAASVHQAQAATTYNAVVSKSSSDGKTFKTIADAIASAPA |
| | | GSTPFVILIKNGVYNERLTITRNNLHLKGESRNGAVIAAATAAGTLKSDGSKW |
| | | GTAGSSTITISAKDFSAQSLTIRNDFDFPANQAKSDSDSSKIKDTQAVALYVTKS |
| | | GDRAYFKDVSLVGYQDTLYVSGGRSFFSDCRISGTVDFIFGDGTALFNNCDLV |
| | | SRYRADVKSGNVSGYLTAPSTNINQKYGLVITNSRVIRESDSVPAKSYGLGRP |
| | | WHPTTTFSDGRYADPNAIGQTVFLNTSMDNHIYGWDKMSGKDKNGNTIWFN |
| | | PEDSRFFEYKSYGAGATVSKDRRQLTDAQAAEYTQSKVLGDWTPTLPGGSLE |
| | | HHHHHH |
| 77 | 34 | MLDMPIDPVYYQLAEYFDSLPKFDQFSSAREYREAINRIYEERNRQLSQHERVE |
| | | RVEDRTIKGRNGDIRVRVYQQKPDSPVLVYYHGGGFVICSIESHDALCRRIARL |
| | | SNSTVVSVDYRLAPEHKFPAAVYDCYDATKWVAENAEELRIDPSKIFVGGDSA |
| | | GGNLAAAVSIMARDSGEDFIKHQILIYPVVNFVAPTPSLLEFGEGLWILDQKIM |
| | | SWFSEQYFSREEDKFNPLASVIFADLENLPPALIITAEYDPLRDEGEVFGQMLRR |
| | | AGVEASIVRYRGVLHGFINYYPVLKAARDAINQIAALLVFDGGSLEHHHHHH |
| 78 | 35 | MTLDLQVQSFLAQGGNLNTLTGEEHGEAKAVFKVEDFYIPVKDGEIKLRVYTP |
| | | NEKESLPVFVYLHGGGWVAGDIQAVDTLCQNIAHDAECIVVAVEYRLAPTHK |
| | | FPVPLEDCYEATKWVAENSSMLNADKTKIAIGGDSAGGNIAAAVVIMAQKFN |
| | | NLSLVAQVLVYPVVDLTLTFKAQSYRDNAEGYLLTTEGVFWATQMYLRDEID |
| | | RYNVFASPSVNNELENLPPALIITAEYDPLRDDGAAYAKRLEAAGIPVEYKCYE |
| | | GMVHGFFWMAGIMDKGLQARLQVSNYLKSVFVGKGGSLEHHHHHH |
| 79 | 36 | MPLDPVIQQVLDQLNRMPAPDYKHLSAQQFRSQQSLFPPVKKEPVAEVREFD |
| | | MDLPGRTLKVRMYRPEGVEPPYPALVYYHGGGWVVGDLETHDPVCRVLAKD |
| | | GRAVVFSVDYRLAPEHKFPAAVEDAYDALQWIAERAADFHLDPARIAVGGDS |
| | | AGGNLAAVTSILAKERGGPALAFQLLIYPSTGYDPAHPPASIEENAEGYLLTGG |
| | | MMLWFRDQYLNSLEELTHPWFSPVLYPDLSGLPPAYIATAQYDPLRDVGKLY |
| | | AEALNKAGVKVEIENFEDLIHGFAQFYSLSPGATKALVRIAEKLRDALAGGSLE |
| | | HHHHHH |
| 80 | 37 | MPLDPEVRNFLQVYYKANIIDFTKYQFQEIRQKVNELLAKAVPKDPVGETRDM |
| | | KIKLEDYELPIRIYSPIKRTNNGLVMHFHGGAWILGSIETEDAISRILSNSCECTVI |
| | | SVDYRLAPEYKFPTAVYDCFNAIVWARDNAGELGIDKDKIATFGISAGGNLVA |
| | | ATSLLARDNKLKLTAQVPVVPFVYLDLASKSMNRYRKGYFLDINLPVDYGVK |
| | | MYIRDEKDLYNPLFSPLIAEDLSNLPQAIVVTAEYDPLRDQGEAYAYRLMESG |
| | | VPTLSFRVNGNVHAFLGSPRTSRQVTVMIGALLKDIFKGSSLEHHHHHH |
| 81 | 38 | MMPLDPRIKELLESGFIVPIGKASVDEVRKIFRQLASAAPKVEVGKVEDIKIPGS |
| | | EANINARVYLPKANGPYGVLIYLHGGGFVIGDVESYDPLCRAITNACNCVVVS |
| | | VDYRLAPEYKFPSAVIDSFDATNWVYNNLDKFDGKMGVAIAGDSAGGNLAA |
| | | VVALLSKGKLNLKYQILIYPAVGFDSVSRSMIEYSDGFFLTREHIEWFGSQYLR |
| | | SPADLLDFRFSPILAQDLSGLPPALIITAEYDPLRDQGEAYANRLLQAGVPVTSV |
| | | RFNNVIHGFLSFFPLIEQGRDAISLIGSVLRRTFYDKSGGSLEHHHHHH |
| 82 | 39 | MIDPKIKKLLESTIQLPIGKASVEEIRSLFKQFSSLTPREEVGKIEDITIPGSETNIK |
| | | ARVYYPKTQGPYGVLVYYHGGGFVLGDIESYDPLCRAITNSCQCVTISVDYRL |
| | | APENKFPAAVVDSFDALKWVYNNSEKFNGKYGIAVGGDSAGGNLAAVTAILS |
| | | KKENIKLKYQVLIYPAVSFDLITKSLYDNGEGFFLTREHIDWFGQQYLRSFADL |
| | | LDFRFSPILADLNDLPPALIITAEHDPLRDQGEAYANKLLQSGVQVTSVRFNNVI |
| | | HGFVSFFPFIEQGRDAIGLIGYVLRKVFYGKGGSLEHHHHHH |
| 83 | 40 | MTEPLIIEPSQPADSAVIWLHGLGADRFDFEPVARLLGQHLPSTRFILPQAPTRP |
| | | VTFNMGHAMPSWYDILALDGSERAINPADLEASSETLIALINAQQQSGIDSKRI |
| | | VLAGFSQGGAVVLHTALLRFDEKLAGVLALSTYAPTFNAETQFAESKQNLPVL |
| | | CMHGSEDAVLPISMGRAVYDKLSEQGIKANWRDYPMGHEVRPEQLRDILDW |
| | | LKNTLPSLPGGSLEHHHHHH |
| 84 | 41 | MDKPIILDPKQSADSAVIWLHGLGATKEDFLPVAQILQRDALPHTRFILPQAPV |
| | | RPVTLNNGFPMPSWYDIIALTSPREIKLSELDESSQSIIALIEAEIEKGIPLERIILA |
| | | GFSQGGAVVLHTAFIAYPKNVGGVMALSTYSATFDEAITLDEKKKQIPTLHLH |
| | | GSLDPVVKIELGRAAEQFLKAQGIDTRWHDYPMQHEVINDELQDIAKWLIERL |
| | | GGGSLEHHHHHH |
| 85 | 42 | MSDTLILEPTHRADACVIWLHGLGADRYDFLPVAEALQDVLGTTRFVLPQAPT |
| | | RAVTINGGWAMPSWYDILAMSPERAIDEAQLEASAQQVMALAQAQVDGGIEP |
| | | RRIFLAGFSQGGAVVLHTAFLRWEDELGGVLALSTYGPTFTDGMTLPDAKRQ |
| | | LPVLCLHGTLDDVVLPAMGRAAHDRLAAAGVPVGWRDYPMAHEVLPQQVR |
| | | DIGAWLVERLHSGGSLEHHHHHH |
| 86 | 43 | MSQPLLLEPTQPADSCVIWLHGLGADRYDFEPVARMLQKVLPRTRFILPQAPT |
| | | RPVTVFNGMPAPSWYDIKAMAPARAIDEAQLDASADAVIALIEGQLAEGIAQR |
| | | RIVLAGFSQGGAVVLHTGYLRWPGELGGVMALSTYGPTFDDDLQLPEAKKQQ |
| | | PALCLHGTYDDVVAPAMGRAAYDFLQRQGVAVQWRDYPMAHEVSNQEIADI |
| | | AAWLRERLGGSLEHHHHHH |
| 87 | 44 | MSEPLILDAPNADACIIWLHGLGADRTDFKPVAEALQMVLPSTRFILPQAPSQA |
| | | VTVNGGWVMPSWYDILAFSPARAIDEDQLNASADQVIALIDEQRAKGIAAERII |
| | | LAGFSQGGAVVLHTAFRRYAQPLGGVLALSTYAPTFDDLALDERHKRIPVLHL |
| | | HGSQDDVVDPALGRAAHDALQAQGVEVGWHDYPMGHEVSLEEIHDIGAWLR |
| | | KRLGGSLEHHHHHH |
| 88 | 45 | MTDPLIIEPAQTADSCVIWLHGLGADRYDFQPVAEMLQQRLLHTRFVLPQAPT |
| | | RAVTINGGWAMPSWYDIQAMSPARAIDQAQLEQSAQTVIELIEQQRDSGIDPR |
| | | RIFLAGFSQGGAVVYHTAFLRWAGPLGGVLALSTYAPTFGDDLKLSPLQAGLP |
| | | VLCLHGSRDDVVPPAMGRAAHDCLQQNQVQTQWKEYPMAHEVQPTEIQDIG |
| | | DWLASRLGGGSLEHHHHHH |
| 89 | 46 | MTEPLILQPAKPADACVIWLHGLGADRYDFMPVAEALQESLLTTRFVLPQAPT |
| | | RPVTINGGYEMPSWYDIKAMSPARSISLEELEVSAKMVTDLIEAQKRTGIDASR |
| | | IFLAGFSQGGAVVFHTAFINWQGPLGGVIALSTYAPTFGDELELSASQQRIPAL |
| | | CLHGQYDDVVQNAMGRSAFEHLKSRGVTVTWQEYPMGHEVLPQEIHDIGAW |
| | | LAARLGGGSLEHHHHHH |

### Molecular Modeling and Design

Designs were built using Compound D as the model's substrate. Compound D was selected to represent the diacylated anthocyanins we sought to transform to Compound I. A conformer library of 5,000 conformations was generated using Spartan (Spartan '14 Wavefunction, Inc. Irvine, CA). This library was docked into the active site of crystal structure model using RosettaDock (Meiler et al., “ROSETTALIGAND: Protein—small molecule docking with full side-chain flexibility,” Proteins: Structure, Function, and Bioinformatics. 65(3):538-548, 2006). To ensure the docked interface were bound in a catalytically competent orientation for the esterase chemistry to occur, catalytic constraints were added to ensure that the catalytic residues (S114, D168, H199, L23, Q115) were within the appropriate distances and angles to one another and to the ester bond of the anthocyanin we aimed to cleave. Eight rounds of design were done, each running the modeling protocol 5,000 times. The top 20 designs from each round were examined in FoldIt and subsets of mutations were chosen for characterization based on the beneficial interactions being introduced.

Of the active enzymes, enzyme 46 of Tables 9 and 10 (referred to herein as 1AUR; corresponding to SEQ ID NO: 89) was amongst the most active enzymes that also had a previously determined crystal structure, making it the top lead from database mining efforts for further optimization. To enhance functionality, we docked Compound F into the crystal structure while employing catalytic constraints to ensure catalytically relevant orientations were modeled. A total of 3,000 models were generated from which the lowest energy was selected to run Rosetta Design simulations around, and the 15 lowest-energy designs were selected to examine manually using FoldIt (Kleffner et al., “Foldit Standalone: a video game-derived protein structure manipulation interface using Rosetta,” Bioinformatics. 33(17): 2765-2767, 2017)). Given the native enzyme's specific activity toward fatty acid chains, we focused on adding polar residues to the active site to promote hydrogen bonding between the enzyme and the highly polar anthocyanins. Focus was placed on residues surrounding the active site, which occurs at the interface of the protein dimer. A total of 53 unique designed protein sequences were chosen over three rounds to characterize for degradation of di-acylated anthocyanins (Table 10). Upon completion of each round of design, residue sites that positively altered activity were targeted for further mutagenesis, with individual residues being replaced by amino acids with similar characteristics. Positive mutations were also combined to probe for combinatorial enhancement of activity.

### Protein Purification and Enzymatic Assay of Esterases

An E. coli codon optimized gene encoding each protein, i.e., one of the enzymes of Table 9, was purchased from Twist Biosciences and transferred into pET29b+ to encode a C-terminal hexahistidine tag (SEQ ID NO: 91). Mutants plasmids were produced by Kunkel mutagenesis (Kunkel et al., “Rapid and efficient site-specific mutagenesis without phenotypic selection,” Proceedings of the National Academy of Sciences. 82(2):488-492, 1985). Plasmids were incorporated into Escherichia coli BL21(DE3) via electroporation. 500 mL cultures were grown in Terrific Broth at 37° C., induced with 1 mM isopropyl-β-D-1-thiogalactopyranoside (IPTG), and allowed to express at 18° C. for 24 hours. Proteins were purified with immobilized metal affinity chromatography and eluted in HEPES buffer pH 8.0 to 1 mg/mL. 90 μL of buffered enzyme was mixed with 10 μL red cabbage extract (RCE) suspended in 1% HCl (100 mg/mL). To detect the presence of activity, assays were allowed to run for 24 at room temperature, after which they were quenched with 5% HCl. Assays to determine the rate of reactions were conducted using an enzyme concentration of 0.5 mg/mL. Reaction products were analyzed by liquid chromatography (HPLC) on an Agilent 1100 Series. The column (Phenomenex pentafluorophenyl propyl 2.6 μm core, 100×4.6 mm) was held at room temperature. The mobile phase was a gradient mixture of solvent A (4.5% formic acid in Milli-Q water) and solvent B (acetonitrile)as follows: 100% A (0 min), 0% to 30% B (25 min), 30% to 95% B (1 min), 95% B (5 min), 95% to 0% B (1 min), 100% A (13 min). Products were monitored at 530 nm, the maximum absorbance of Compound I. The experiment was repeated for enzymes that demonstrated activity on RCE anthocyanins. For these enzymes, the reaction size was increased 5-fold, and a portion was withdrawn and quenched at timed intervals in order to evaluate the rate of the reactions.

### Gram Scale Protein Production & Purification

The M73H point-mutant was created from the 1AUR Wild type plasmid via Kunkel Mutagenesis. The sequence of the M73H point-mutant corresponds to the amino acid sequence of SEQ ID NO: 16 tagged with a hexahistidine tag of SEQ ID NO: 90 attached at the C-terminus of the protein. Then transformed into chemically competent Escherichia coli BLR cells and plated on agar (Fisher BP1423500) plates containing 50 μg/mL kanamycin (Fisher AC450810500). Single colonies were picked and used to inoculate 10 mL Terrific Broth (TB) (Fisher BP2468-2) in 50 mL Falcon tubes (Fisher 14-959-49A). These were incubated at 37° C. while shaking at 300 RPM for 12-16 hours. For long term storage of the inoculated cells, a glycerol stock was made with 500 μL of the cell culture and 500 μL of 50% autoclaved glycerol (Fisher G33-4) in Mili-Q water and stored in −80° C. freezer. To begin protein production, the starting culture consisted of a 50 mL Terrific Broth (TB) with 50 μg/mL kanamycin and 15 μg/mL tetracycline hydrochloride (Fisher 5834625GM) in a 250 mL Erlenmeyer baffled flask. This was inoculated with a swab of cells from the glycerol stock and shaken at 250 RPM at 37° C. After 14-16 hours, the starter culture was poured into a 1 L Erlenmeyer baffled flask containing 450 mL TB with 50 μg/mL kanamycin and 15 μg/mL tetracycline hydrochloride.

These flasks were covered with breathable seals (Fisher 12-567-05) and shaken at 300 RPM, at 37° C. for 4-6 hours until the OD₆₀₀ reached 0.7-1.0. The 500 mL cultures were then cooled at 4° C. for 30 minutes, and enough isopropyl-β-D-1-thiogalactopyranoside (IPTG) (Fisher 420322-25GM) was added for a final concentration of 1 mM and shaken again at 300 RPM, at 18° C. for 24 hours to induce expression of the M73H mutant enzyme. After expression, the cultures were pelleted by being spun down for 10 minutes at 4700 RPM at 4° C. The supernatant was poured out and the pelleted cells were resuspended in 10 mL 1× phosphate buffered saline (PBS) buffer pH 7.4 (135 mM NaCl, 2.7 mM KCl, 4.3 mM Na₂HPO₄, and 1.4 mM KH₂PO₄) (diluted from pre-made 20×PBS buffer (VWR Int. 100218-160)) and stored in 50 mL falcon tubes at −20° C. For purification, the frozen cells containing enzyme were thawed on ice and brought up to a volume of 40 mL with 1×PBS buffer. Then sonicated at an amplitude of 50 with a process time of 3 minutes, with 30 seconds on and 30 seconds off. The lysed cells were spun down for 1 hour at 4700 RPM, at 4° C. The supernatant lysate is then collected and used for the reaction.

### Gram Scale Peak B Enzyme Reaction & Product Validation

To determine the yield of enzyme in the lysate, 500 μL of lysate was purified from every batch. A 1 mL column with 200 μL Cobalt beads (Fisher PI89966) was washed with 1 mL Wash buffer pH 7.4 (same as the 1×PBS buffer with 10 mM Imidazole (Fisher AC122020020) added). Then 500 μL of lysate was poured through the column, the His-tagged M73H mutant enzyme became immobilized on the beads via metal ion affinity chromatography and washed with 5 mL of Wash buffer. Enzyme was eluted with 250 μL Elution buffer pH 7.4 (same as the 1×PBS buffer with 200 mM Imidazole added) and protein yield was determined by A280 using a BioTek Epoch spectrophotometer.

To set up the reaction, the substrate, Red Cabbage (RC-B), was dissolved in 1×PBS buffer pH 7.4 at 50 mg/mL and brought up to pH 7.5 with 6M NaOH (Fisher S318-500) in a 1 L glass bottle. This was combined with an appropriate amount of lysate for a final ratio of 1 mg of enzyme for every 200 mg of RC-B substrate. For example, 150 mL of lysate at 1 mg/mL of enzyme would be combined with 30 g RC-B substrate dissolved in 600 mL of 1×PBS. The assay reaction proceeded for 4 days in a 1 L Nalgene Amber bottle (Thermo 967-10936-357), all while gently agitated on a rocker (Neta BMK-B3D1308). The reaction was then quenched by bring down the pH to 2.8 with 6M HCl (Fisher BP1756100) and then frozen at −20° C. To verify reaction completion, 500 μL of product was combined with 500 μL of Acetonitrile (Millipore Sigma 439134-4L) in a 2 mL centrifuge tube and spun down at 10,000 RPM for 10 minutes at 4° C. Then 100 μL of the supernatant was placed in a vial (Fisher 0339115) and run on a High-Performance Liquid Chromatography (HPLC) machine at a wavelength of 530 nm to check for reaction completion. The HPLC run method begins by injecting 2 μL of sample, then starts a flow rate of 0.5 mL/min consisting of 4.5% formic acid (Millipore Sigma 27001-500ML-R) in Mili-Q water. For the first 25 minutes, the flow of 4.5% formic acid was slowly decreased to 70%, while acetonitrile was slowly increased to 30% of the mobile phase. This continued until the acetonitrile and 4.5% formic acid reached 95% and 5% of the mobile phase, respectively, at 26 minutes. The mobile phase stayed constant until 31 minutes into the run. At 32 minutes, the acetonitrile decreased back down to 0%, while the 4.5% formic acid increased back up to being 100% of the mobile phase. This continued until the end of the run at 45 minutes. During the run, the column was at 20° C., and was a Phenomenex Kinetex PFP column, particle size of 2.6 μm, pore size of 100 Å, and the dimensions were 100×4.6 mm.

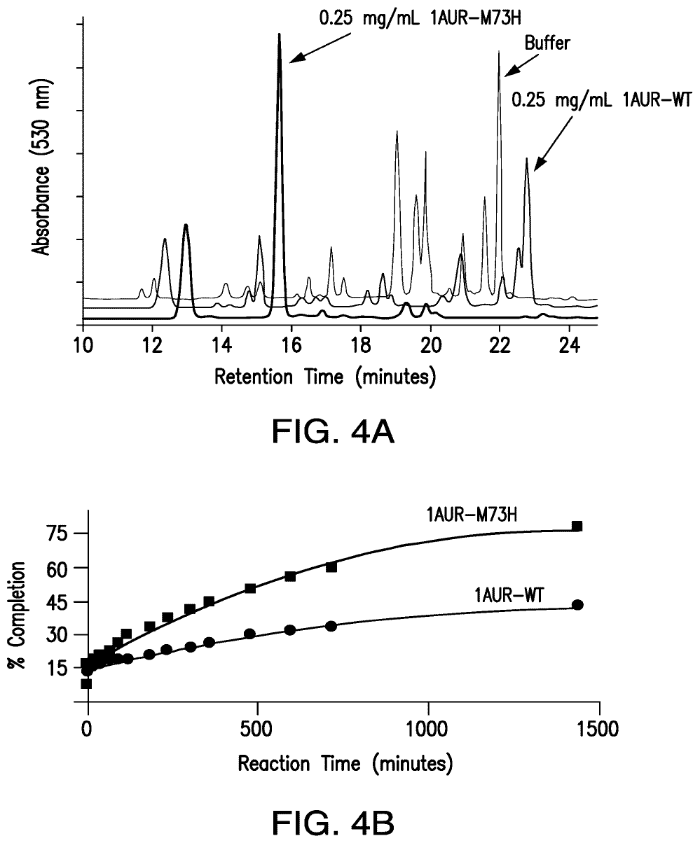

It was observed that the mutant with the best conversion rates of Compounds A-F to Compounds I and II was the M73H mutant of 1AUR, which yielded 84% more Compound I than the native enzyme over 24 hours (FIGS. 4A and 4B).

### Example 2: Isolation of Compound I

The present Example provides for a protocol to isolate Compound I from a red cabbage juice and/or extract that has undergone enzymatic treatment described herein.

First, the enzymes were precipitated from the reaction mixture. Hydrochloric acid was added to the reaction mixture to adjust the pH to about 1.10, and the reaction mixture was left to sit in an ice bath for about 2 hours. The liquid was then transferred to centrifuge tubes and centrifuged at 3540 rcf for 5 minutes. After this time, the supernatant was decanted and subsequently vacuum filtered through a Buchner funnel and Whatman 1 filter paper.

The resulting material was then further purified by Solid Phase Extraction using a Phenomenex Strata C18 SPE cartridge. The cartridge was activated with 2 column volumes of 190 proof ethanol, followed by 2 column volumes of acidified water (0.01% HCl). The material was then loaded onto the cartridge. The SPE cartridge was washed with 2 column volumes of acidified water, then the anthocyanin compounds were eluted using acidified ethanol (0.01% HCl).

The ethanol was then removed from the solution containing the anthocyanins by placing the solution in a warm water bath at 34° C. with a steady stream of nitrogen gas passing over. For every 10 mL of ethanol evaporated, an equal volume of Milli-Q 18.2 MOhm deionized water was added to the sample.

Compound I was then purified by using reverse phase preparatory HPLC-DAD. The HPLC-DAD parameters were as follows: 250 mm L×50 mm D Phenomenex Luna C18 (2) preparatory LC column with 10 micron particle size and 100 A pore size; mobile phases A: 5% acetic acid in deionized water and B: 5% acetic acid in 190 proof ethanol; flow rate of 60 mL/min; wavelength detection set to 280 nm and 520 nm. The gradient method used is: (1) B: 0%-12%, 0-30 min. (2) B: 12%-50%, 30-35 min. (3) B: 50% isocratic, 35-45 min. (4) B: 50%-0%, 45-46 min. Fractions were collected manually based on the elution of Compound I observed using visible signal, 520 nm.

### Example 3: Lyophilization of Compound I

The present Example provides for procedures used to lyophilize Compound I. Fractions comprising Compound I as isolated in Example 2 were collected. Ethanol was removed from the combined fraction using rotary evaporation, where the water bath was set to 37° C. and the pressure was 60 mbar. These conditions allowed for removal of ethanol, as well as some acetic acid and water.

The resulting aqueous sample was transferred to a polypropylene container and immersed in liquid nitrogen until fully frozen. The container was then placed into the lyophilizer with pressure set to 0.03 mbar and the condenser temperature set to −80° C.

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods or steps.

Patents, patent applications publications product descriptions, and protocols are cited throughout, the disclosures of which are incorporated herein by reference in their entireties for all purposes.

What is claimed is:

1. A method for selectively converting one or more diacylated anthocyanins into a monoacylated anthocyanin product, the method comprising:
a) providing a fruit or vegetable juice or extract comprising a diacylated anthocyanin or a mixture of anthocyanins, wherein the mixture of anthocyanins comprises one or more diacylated anthocyanins and, optionally, one or more monoacylated anthocyanins; and
b) subjecting the fruit or vegetable juice or extract to enzymatic treatment by one or more enzymes, wherein the one or more enzymes selectively hydrolyzes the one or more diacylated anthocyanins into the monoacylated anthocyanin product.

2. The method of claim 1, wherein the fruit or vegetable juice or extract is red cabbage juice or extract.

3. The method of claim 1, wherein the monoacylated anthocyanin product is Compound I having the following structure:

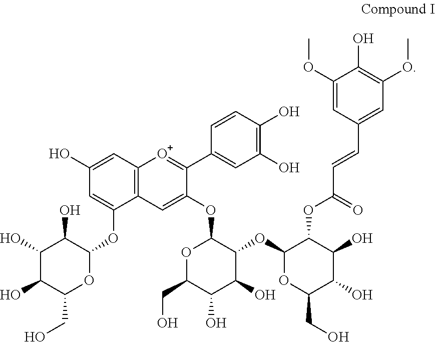 [SMILES: COc1cc(/C=C/C(=O)O[C@H]2[C@H](O[C@H]3[C@H](Oc4cc5c(O[C@@H]6O[C@H](CO)[C@@H](O)[C@H](O)[C@H]6O)cc(O)cc5[o+]c4-c4ccc(O)c(O)c4)O[C@H](CO)[C@@H](O)[C@@H]3O)O[C@H](CO)[C@@H](O)[C@@H]2O)cc(OC)c1O]

4. The method of claim 1, wherein the mixture of anthocyanins comprises the one or more monoacylated anthocyanins, and the enzymatic treatment additionally converts the one or more monoacylated anthocyanins into a nonacylated anthocyanin product.

5. The method of claim 4, wherein the nonacylated anthocyanin product is Compound II having the following structure:

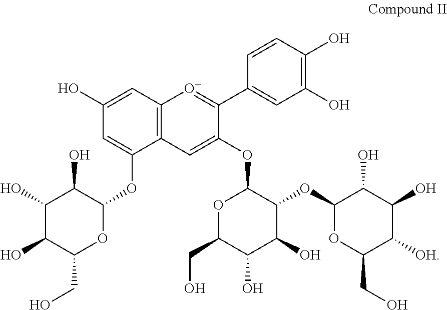 [SMILES: OC[C@H]1O[C@@H](Oc2cc(O)cc3[o+]c(-c4ccc(O)c(O)c4)c(O[C@@H]4O[C@H](CO)[C@@H](O)[C@H](O)[C@H]4O[C@@H]4O[C@H](CO)[C@@H](O)[C@H](O)[C@H]4O)cc23)[C@H](O)[C@@H](O)[C@@H]1O]

6. The method of claim 1, wherein the one or more enzymes comprises one or more esterases.

7. The method of claim 6, wherein the one or more esterases is a naturally occurring esterase.

8. The method of claim 7, wherein the naturally occurring esterase comprises the amino acid sequence of any one of SEQ ID NO: 1-15.

9. The method of claim 6, wherein the one or more esterases is a modified esterase.

10. The method of claim 9, wherein the modified esterase is a modified carboxylesterase from Pseudomonas fluorescens or a modified carboxylesterase from Chromohalobacter salexigens.

11. The method of claim 10, wherein the modified carboxylesterase from P. fluorescens comprises one or more amino acid substitutions at position number 25, 28, 29, 30, 31, 38, 39, 63, 65, 70, 73, 200, or 201 relative to a naturally occurring carboxylesterase from P. fluorescens.

12. The method of claim 10, wherein the modified carboxylesterase from C. salexigens comprises one or more amino acid substitutions at amino acid position number 23, 28, 75, 115, 117, 119, 121, 122, 125, 129, 168, 171, 173, 202, 209, or 212 relative to a naturally occurring carboxylesterase from C. salexigens.

13. The method of claim 9, wherein the modified esterase comprises the amino acid sequence of any one of SEQ ID NO: 16-43.

14. The method of claim 1, wherein the monoacylated anthocyanin product is Compound I, and Compound I is isolated from the solution following the enzymatic treatment by a purification process comprising one or more of enzyme precipitation, solid-phase extraction, or preparatory high performance liquid chromatography (HPLC).

15. The method of claim 14, wherein Compound I is isolated by a purification process comprising sequentially performing the steps of:
(i) enzyme precipitation;
(ii) solid-phase extraction; and, optionally,
(iii) preparatory HPLC,
wherein the preparatory HPLC of step (iii) is performed if the solution comprises two or more anthocyanins.

16. A method of selectively hydrolyzing an anthocyanin of Formula 1: 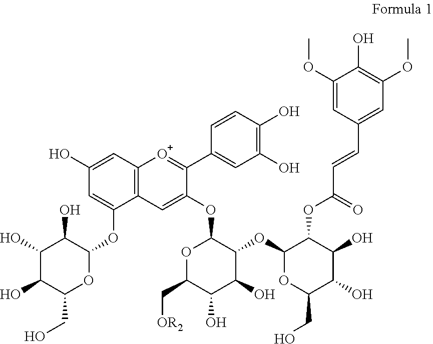
wherein R₁ is H or an acyl group, and
R₂ is an acyl group that is the same or different from the acyl group of R₁;
the method comprising contacting a solution comprising the anthocyanin of Formula 1 with one or more enzymes,
wherein the enzyme selectively hydrolyzes the anthocyanin to remove the acyl group of R₂ and does not remove R₁.

17. The method of claim 16, wherein the acyl group of R₁ and/or R₂ has the structure 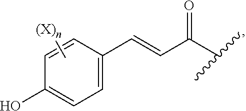
wherein X is a C₁₋₄ alkoxy group, and
n is an integer from 0-2.

18. The method of claim 16, wherein the one or more enzymes comprise the amino acid sequence of any one of SEQ ID NO: 1-43.

19. The method of claim 16, wherein the solution is a fruit or vegetable juice or extract.

20. The method of claim 16, wherein the selective hydrolysis produces Compound I and/or Compound II:

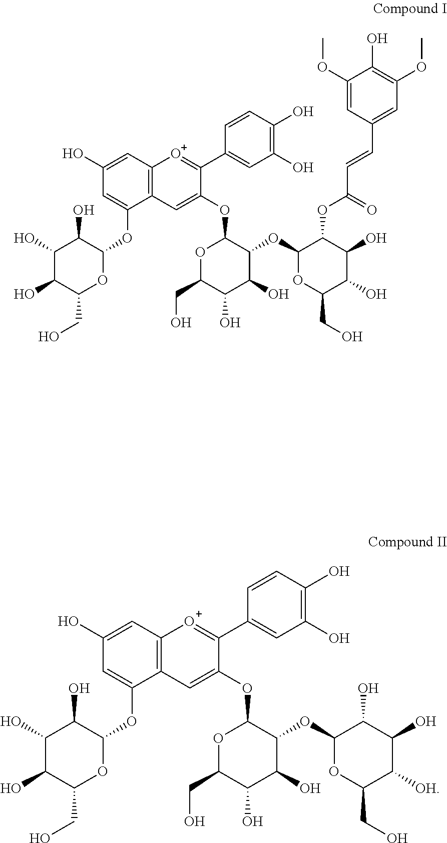 [SMILES: COc1cc(/C=C/C(=O)O[C@H]2[C@H](O[C@H]3[C@H](Oc4cc5c(O[C@@H]6O[C@H](CO)[C@@H](O)[C@H](O)[C@H]6O)cc(O)cc5[o+]c4-c4ccc(O)c(O)c4)O[C@H](CO)[C@@H](O)[C@@H]3O)O[C@H](CO)[C@@H](O)[C@@H]2O)cc(OC)c1O ; OC[C@H]1O[C@@H](Oc2cc(O)cc3[o+]c(-c4ccc(O)c(O)c4)c(O[C@@H]4O[C@H](CO)[C@@H](O)[C@H](O)[C@H]4O[C@@H]4O[C@H](CO)[C@@H](O)[C@H](O)[C@H]4O)cc23)[C@H](O)[C@@H](O)[C@@H]1O]

21. The method of claim 20, wherein Compound I is present in an amount of from about 40% to about 100% by weight of a total anthocyanin content of the solution at least about 24 hours after the solution is contacted with the enzyme.

22. The method of claim 16, wherein Compound I is isolated from the solution following the selective hydrolysis by a purification process comprising one or more of enzyme precipitation, solid-phase extraction, or preparatory HPLC.

23. The method claim 22, wherein Compound I is isolated by a purification process comprising sequentially performing the steps of:
(i) enzyme precipitation;
(ii) solid-phase extraction; and, optionally,
(iii) preparatory HPLC,
wherein the preparatory HPLC of step (iii) is performed if the solution comprises two or more anthocyanins.

24. An enzyme comprising the amino acid sequence of any one of SEQ ID NO: 16-43.
